(19) United States
(12) Patent Application Publication
(10) Pub. No.: US 2025/0205337 A1
(43) Pub. Date: Jun. 26, 2025

(54) COMPOSITIONS AND METHODS FOR ACTIVATING IMMUNE CELLS

(71) Applicant: MDX Management LLC, La Jolla, CA (US)

(72) Inventors: Yuan LIU, Decatur, GA (US); Zhen BIAN, Tucker, GA (US); Lei SHI, Decatur, GA (US); Harry STYLLI, La Jolla, CA (US)

(21) Appl. No.: 18/852,933

(22) PCT Filed: Mar. 30, 2023
(86) PCT No.: PCT/US2023/017002

§ 371 Date: Sep. 30, 2024

Related U.S. Application Data
(60) Provisional application No. 63/325,439, filed on Mar. 30, 2022.

(51) Int. Cl. A61K 40/17; A61K 40/24; A61K 40/42; A61P 35/00; C12N 5/0783; C12N 5/0786

(52) U.S. Cl. CPC A61K 40/17; A61K 40/24; A61K 40/4201; A61P 35/00; C12N 5/0636; C12N 5/0645; A61K 2239/54; C12N 2501/22; C12N 2501/2304; C12N 2501/2306; C12N 2501/231; C12N 2501/24; C12N 2501/25; C12N 2502/1114

(57) ABSTRACT

The present application provides compositions and methods for producing antigen presenting cells (APCs) from monocytes (e.g., monocytes from cancer patients) that involve an IL-10 receptor activator (e.g., IL-10), IFNγ receptor activator (e.g., IFNγ), TNFα receptor activator (e.g., TNFα), IL-4 receptor activator (e.g., IL-4), GM-CSF receptor activator (e.g., GM-CSF), and/or IL-6 receptor activator (e.g., IL-6). APCs produced accordingly are also provided, as well as methods of activating immune cells (e.g., T cells) via co-culturing with APCs. The activated immune cell compositions and the methods of treatments that involve the activated immune cells are also provided.

### CROSS-REFERENCE TO RELATED APPLICATION

This application claims priority to U.S. provisional application 63/325,439, filed on Mar. 30, 2022, the content of which is incorporated by reference in its entirety for all purposes.

### FIELD OF THE INVENTION

The present invention relates to compositions and methods for a) promoting survival of monocytes, b) promoting differentiation and/or maturation of antigen presenting cells (APCs) derived from monocytes (such as monocytes from cancer patients or healthy donors), and c) activating immune cells (such as T cells).

### BACKGROUND OF THE INVENTION

Professional antigen presenting cells (APCs), e.g., dendritic cells and macrophages, and their mediated network of immunogenic immunity are central components for establishing the antigen-specific adaptive immunity that protects the host from cancerous mutations, infectious pathogens and injuries. In cancer immunotherapy, the promises of APCs are their abilities to conduct phagocytosis towards tumor cells and then present antigens to activate tumor-specific adaptive immunity, including tumoricidal T cells and long-lasting anticancer antibodies. In addition, successful production of APCs also would promote the development of APC-vaccines to treat infections such as those caused by virus, bacteria and other pathogens. With these capacities, APC-based therapies once established is expected to achieve a complete cancer-cure efficacy, both systemically eliminating tumors and metastases and establishing an immune memory that prevents recurrence/relapse.

However, the use of APCs for cancer treatment over the years has uncovered several serious limitations. For example, isolation and ex vivo stimulation of autologous APCs proved time-consuming, expensive, and the quality of ex vivo-generated DCs can be variable. The use of patient-derived autologous DCs therefore limits standardization of DC-based treatment protocols. See e.g., Eggermont et al., Trends Biotechnol. 2014 September; 32 (9): 456-65. Among them, the one most critical barrier of APC therapy lies in the absence of technology that robustly drives monocytes from cancer patients to differentiate into proinflammatory DC/macrophage-like, effective APCs.

The disclosures of all publications, patents, patent applications and published patent applications referred to herein are hereby incorporated herein by reference in their entirety.

### BRIEF SUMMARY OF THE INVENTION

The present application in one aspect provides a method of stimulating a population of monocytes from an individual to produce a population of antigen presenting cells (“APCs”), comprising contacting the population of monocytes with a plurality of survival, differentiation and/or maturation factors (“S/D/M factors”) separately or simultaneously, wherein the plurality of S/D/M factors comprise: 1) an IL-10 receptor (IL-10R) activator and 2) one or more agents selected from the group consisting of: an IL-4 receptor (IL-4R) activator, a TNFα receptor (TNFR) activator, and an interferon γ (IFNγ) receptor (IFNGR) activator, thereby obtaining a population of APCs. In some embodiments, the IL-10R activator is selected from the group consisting of: an IL-10 (e.g., a pegylated IL-10, e.g., pegilodecakin or AM0010), an IL-10 family member (e.g., IL-19, IL-20, IL-22, IL-24, IL-26, IL-28), an IL-10R agonist antibody, a small molecule activator of IL-10R, and an activator of the IL-10R downstream STAT3 (e.g. Long noncoding RNA (LncRNA) PVT1, NEAT1, FEZF1-AS1, UICC). See e.g., Yang et al., Cytokine Growth Factor Rev. 2019 October; 49:10-22. In some embodiments, the IL-10R activator is IL-10. In some embodiments, the IL-10 is a human IL-10 or a human recombination IL-10. In some embodiments, the IL-10 is present in the medium at a concentration of at least about 2 ng/ml, optionally at least about 10 ng/ml, further optionally about 10 ng/ml to about 200 ng/ml (e.g., about 20 ng/ml).

In some embodiments according to any of the methods described above, the plurality of S/D/M factors comprise an IL-4R activator, optionally wherein the IL-4R activator is selected from the group consisting of IL-4, IL-13, an IL-4R agonist antibody, and a small molecule activator of IL-4R. In some embodiments, the IL-4R activator is IL-4. In some embodiments, the IL-4 is a human IL-4 or a human recombinant IL-4. In some embodiments, the IL-4R activator is IL-13. In some embodiments, the IL-13 is a human IL-13 or a human recombinant IL-13. In some embodiments, the IL-4 is present in the medium at a concentration of at least about 15 μg/ml, optionally at least about 30 μg/ml, further optionally about 30 μg/ml to about 1 ng/ml (e.g., about 100 μg/ml to about 1 ng/ml). In some embodiments, the IL-13 is present in the medium at a concentration of at least about 30 μg/ml, optionally at least about 60 μg/ml, further optionally about 60 μg/ml to about 2 ng/ml (e.g., about 100 μg/ml to about 2 ng/ml).

In some embodiments according to any of the methods described above, the plurality of S/D/M factors comprise a TNFR activator, optionally wherein the TNFR activator is selected from the group consisting of TNFα, a TNFR agonist antibody, and a small molecule activator of TNFR. In some embodiments, the TNFR activator is TNFα. In some embodiments, the TNFα is a human TNFα or a human recombinant TNFα. In some embodiments, the TNFα is present in the medium at a concentration of at least about 0.5 ng/ml, optionally at least about 1 ng/ml, further optionally about 0.5 ng/ml to about 30 ng/ml (e.g., about 1-10 ng/ml).

In some embodiments according to any of the methods described above, the plurality of S/D/M factors comprise an IFNGR activator, optionally wherein the IFNGR activator is selected from the group consisting of IFNγ, an IFNGR agonist antibody, and a small molecule activator of IFNGR. In some embodiments, the IFNGR activator is IFNγ. In some embodiments, the IFNγ is a human IFNγ or a human recombinant IFNγ. In some embodiments, the IFNγ is present in the medium at a concentration of at least about 5 ng/ml, optionally at least about 10 ng/ml, further optionally about 10 ng/ml to about 200 ng/ml (e.g., about 50-100 ng/ml).

In some embodiments according to any of the methods described above, the plurality of S/D/M factors are present in a single composition.

In some embodiments according to any of the methods described above, at least one of the plurality of S/D/M factors is provided separately from one of other S/D/M factors in the plurality of S/D/M factors.

In some embodiments according to any of the methods described above, the plurality of S/D/M factors comprise two or more agents selected from the group consisting of an IL-4R activator, a TNFR activator, and an IFNGR activator. In some embodiments, the plurality of S/D/M factors comprises IL-10, IL-4, TNFα, and IFNγ.

In some embodiments according to any of the methods described above, the plurality of the S/D/M factors further comprise a GM-CSF receptor (GM-CSFR) activator. In some embodiments, the GM-CSFR activator is selected from the group consisting of GM-CSF, a GM-CSFR agonist antibody, and a small molecule activator of GM-CSFR. In some embodiments, the GM-CSFR activator is GM-CSF. In some embodiments, the GM-CSF is a human GM-CSF or a human recombinant GM-CSF. In some embodiments, the GM-CSF is present in the medium at a concentration of at least about 30 μg/ml, optionally at least about 50 μg/ml, further optionally about 100 μg/ml to about 1 ng/ml (e.g., about 100 μg/ml to about 500 μg/ml, e.g., about 300 μg/ml).

In some embodiments according to any of the methods described above, the plurality of the S/D/M factors further comprise an IL-6 receptor (IL-6R) activator, optionally wherein the IL-6R activator is selected from the group consisting of IL-6, an IL-6R agonist antibody, and a small molecule activator of IL-6R. In some embodiments, the IL-6R activator is IL-6. In some embodiments, the IL-6 is a human IL-6 or a human recombinant IL-6. In some embodiments, the IL-6 is present in the medium at a concentration of at least about 1 μg/ml, optionally at least about 5 μg/ml, further optionally about 5 μg/ml to about 100 μg/ml (e.g., about 10-50 pg/ml, e.g., about 30 μg/ml).

In some embodiments according to any of the methods described above, the plurality of S/D/M factors are derived from a culture of T cells after being treated with anti-CD3 and anti-CD28 antibodies, optionally the plurality of S/D/M factors are derived from the supernatant of the culture. In some embodiments, the T cells are isolated from PBMC of the same individual or a different individual. In some embodiments, the T cells are CD4 T cells. In some embodiments, the T cells are CD8 T cells. In some embodiments, the T cells have not been previously treated with anti-CD3 and/or anti-CD28 antibodies prior to the treatment. In some embodiments, the T cells have been previously treated with anti-CD3 and/or anti-CD28 antibodies prior to the treatment. In some embodiments, the plurality of S/D/M factors are derived from the culture after the T cells are treated with anti-CD3 and anti-CD28 antibodies for about 1-3 days, optionally for about 2 days.

In some embodiments according to any of the methods described above, the monocytes are cultured for at least about 2 days (e.g., about 2-4 days, about 2-3 days, about 2 days) in the presence of the S/D/M factors or the medium derived from the culture of T cells.

In some embodiments according to any of the methods described above, the method further comprises contacting the population of monocytes with a plurality of expansion refinement factors selected from the group consisting of type-I interferon, IFNγ, TNFα, a TLR ligand, CD40L or a CD40-ligating antibody, an anti-PD-L1 antibody, and TPI-1, optionally wherein the type-I interferon comprises IFNα and/or IFNβ, and optionally wherein the TLR ligand is poly IC, CpG, or LPS. In some embodiments, the plurality of refinement factors are provided after the plurality of monocytes are contacted with the plurality of S/D/M factors or the medium derived from the culture of T cells, thereby producing the population of APCs, and wherein the population of APCs are cultured for about 1-5 days in the presence of the plurality of the refinement factors, optionally wherein the population of APCs are cultured for about one day. In some embodiments, the plurality of refinement factors are provided when a) at least about 50% of the monocytes survive, b) at least about 30% of the population of the APCs exhibit a dendritic cell morphology and/or c) the population of APCs express i) a high level of one or more molecules selected from the group consisting of MHC I, MHC II, CD80, CD86, and/or CD40, and/or ii) a low level of SIRPα. In some embodiments, the refinement factors comprise IFNα, IFNγ, and TNFα. In some embodiments, the refinement factors further comprise poly IC, CpG, CD40L, and an anti-PD-L1 antibody.

The present application in another aspect provides a method of promoting the survival of a population of monocytes from an individual in an in vitro culture, comprising cultivating the population of monocytes in a medium having one or more molecules that promote IL-10 receptor (IL-10R) expression on the monocytes. In some embodiments, the one or more molecules comprises an IL-10R activator, optionally wherein the IL-10R activator is selected from the group consisting of: an IL-10, an IL-10R agonist antibody, and a small molecule activator of IL-10R, further optionally the IL-10R activator is IL-10.

The present application in another aspect provides a method of promoting the survival of a population of monocytes from an individual in an in vitro culture, comprising cultivating the population of monocytes in a medium having an IL-10R activator, optionally wherein the IL-10R activator is selected from the group consisting of: an IL-10, an IL-10R agonist antibody, and a small molecule activator of IL-10R, further optionally the IL-10R activator is IL-10.

In some embodiments according to the methods of promoting survival of a population of monocytes discussed above, the population of monocytes express a low level of IL-10R prior to contacting with the molecule.

In some embodiments according to the methods of promoting survival of a population of monocytes discussed above, the culture comprise a TNFα receptor (TNFR) activator, and/or an interferon γ (IFNγ) receptor (IFNGR) activator, optionally wherein the TNFR activator is selected from the group consisting of TNFα, a TNFR agonist antibody, and a small molecule activator of TNFR, and optionally wherein the IFNGR activator is selected from the group consisting of IFNγ, an IFNGR agonist antibody, and a small molecule activator of IFNGR, and further optionally the culture comprises TNFα and/or IFNγ.

The present application in another aspect provides a method of increasing expression of IL-10 receptor (IL-10R) in a population of monocytes from an individual having cancer, comprising contacting the population of monocytes with one or more agents selected from the group consisting of: an IL-10R activator, a TNFR activator, and an IFNGR activator.

The present application in another aspect provides a method of promoting the survival of a population of monocytes from an individual in an in vitro culture, comprising cultivating the population of monocytes in a medium comprising IL-10, TNFα, and IFNγ.

The present application in another aspect provides a method of promoting the differentiation of a population of monocytes from an individual to antigen presenting cells (“APCs”) in an in vitro culture, comprising cultivating the population of monocytes in a medium having one or more molecules selected from the group consisting of an IL-4 receptor (IL-4R) activator, a TNFα receptor (TNFR) activator, and an interferon γ (IFNγ) receptor (IFNGR) activator. In some embodiments, the culture further comprises an IL-6 receptor (IL-6R) activator and/or a GM-CSF receptor (GM-CSFR) activator.

In some embodiments according to any of the methods discussed above, the plurality of monocytes are obtained from the peripheral blood of the individual, optionally wherein the monocytes express CD14 wherein they are obtained from the peripheral blood.

In some embodiments according to any of the methods discussed above, the individual has a cancer. In some embodiments, the individual has a late stage cancer. In some embodiments, the individual has a solid tumor.

In some embodiments according to any of the methods discussed above, the individual has inoperable tumor and/or metastases.

In some embodiments according to any of the methods discussed above, the individual is a human.

The present application in another aspect provides a population of APCs produced by any of the methods relating to producing a population of APCs discussed above. In some embodiments, the APCs express a low level of an inhibitory signaling molecule, wherein the inhibitory signaling molecule is selected from the group consisting of: TGFβR, SIRPα, LIIRBs and Siglec 10.

The present application in another aspect provides a population of APCs, wherein the APCs express a higher level of one or more antigen presentation molecule, wherein the antigen presentation molecule is selected from the group consisting of: MHCI, MHCII, CD86, CD80, OX40L, ICAML, ICOSL, and CD40 than dendritic cells obtained from a healthy human and cultured with GM-CSF and IL-4 for about 5 days, optionally wherein the APCs are produced from monocytes in an ex vivo cell culture, further optionally wherein the monocytes are obtained from a cancer patient. In some embodiments, the APCs express a low level of an inhibitory signaling molecule, wherein the inhibitory signaling molecule is selected from the group consisting of: TGFβR, SIRPα, LIIRBs and Siglec 10.

The present application in another aspect provides a method of activating a population of immune cells, comprising co-culturing the population of immune cells with the population of the APCs of any one of the populations of APCs discussed above, wherein the APCs are pre-loaded with one or more neoantigen peptides. In some embodiments, the method comprises contacting the APCs with a composition comprising a plurality of neoantigen peptides, and/or the APCs have been pre-incubated with the composition. In some embodiments, the composition comprising a plurality of neoantigen peptides is a surgical resection of tumor tissue or a biopsy extract thereof. In some embodiments, the composition comprising a plurality of neoantigen peptides is a mixture of tumor cells or extract thereof isolated from tumor tissue or biopsy. In some embodiments, the composition comprising a plurality of neoantigen peptides is a mixture of isolated neoantigen peptides. In some embodiments, the isolated neoantigen peptides are synthetic peptides. In some embodiments, the APCs are allowed to be in contact with the composition comprising a plurality of neoantigen peptides for about 4 to about 24 hours. In some embodiments, the immune cells are selected from the group consisting of PBMC, tumor infiltrating T cells (TIL), and T cells, optionally the T cells are CD8 T cells and/or CD4 T cells. In some embodiments, the co-culturing was carried out for at least 24 hours. In some embodiments, the method further comprises expanding the population of immune cells following the co-culturing step. In some embodiments, expanding the population of immune cells comprises contacting the immune cells with a cytokine selected from the group consisting of IL-2, IL-7, and IL-15, optionally for about 2 to about 10 days. In some embodiments, the population of immune cells and the antigen presenting cells are derived from the same individual. In some embodiments, the population of immune cells and the antigen presenting cells are not derived from the same individual.

The present application in another aspect provides a population of activated immune cells obtained by any of the methods of activating a population of immune cells discussed above.

The present application in another aspect provides a method of treating cancer in a patient, comprising administering to the patient a population of APCs and/or activated immune cells according to any of the population of APCs and/or activated immune cells described above. In some embodiments, the APCs or activated immune cells are administered intratumorally, intraperitoneally, or intravenously. In some embodiments, the activated immune cells are administered at about 10⁷ to 10⁹ cells per dose. In some embodiments, the method further comprises treating the patient with chemotherapy, radiation therapy, or an immune checkpoint inhibitor. In some embodiments, the method comprises treating the patient with irradiation. In some embodiments, the site of irradiation is different from the site of the cancer to be treated. In some embodiments, the APCs or activated immune cells administered to the patient are derived from the patient. In some embodiments, the APCs or activated immune cells administered to the patient are not derived from the patient. In some embodiments, the cancer to be treated is a solid tumor.

The present application in another aspect provides a composition comprising a plurality of survival, differentiation and/or maturation factors (“S/D/M factors”), wherein the plurality of S/D/M factors comprise: 1) an IL-10 receptor (IL-10R) activator and 2) one or more agents selected from the group consisting of: an IL-4 receptor (IL-4R) activator, a TNFα receptor (TNFR) activator, and an interferon γ (IFNγ) receptor (IFNGR) activator. In some embodiments, the IL-10R activator is selected from the group consisting of: an IL-10, an IL-10R agonist antibody, and a small molecule activator of IL-10R. In some embodiments, the IL-10R activator is IL-10. In some embodiments, the plurality of S/D/M factors comprise an IL-4R activator, optionally wherein the IL-4R activator is selected from the group consisting of IL-4, IL-13, an IL-4R agonist antibody, and a small molecule activator of IL-4R. In some embodiments, the IL-4R activator is IL-4. In some embodiments, the plurality of S/D/M factors comprise a TNFR activator, optionally wherein the TNFR activator is selected from the group consisting of TNFα, a TNFR agonist antibody, and a small molecule activator of TNFR. In some embodiments, the TNFR activator is TNFα. In some embodiments, the plurality of S/D/M factors comprise an IFNGR activator, optionally wherein the IFNGR activator is selected from the group consisting of IFNγ, an IFNGR agonist antibody, and a small molecule activator of IFNGR. In some embodiments, the IFNGR activator is IFNγ. In some embodiments, the plurality of S/D/M factors comprise two or more agents selected from the group consisting of an IL-4R activator, a TNFR activator, and an IFNGR activator. In some embodiments, the plurality of S/D/M factors comprises IL-10, IL-4, TNFα, and IFNγ. In some embodiments, the plurality of the S/D/M factors further comprise a GM-CSF receptor (GM-CSFR) activator. In some embodiments, the GM-CSFR activator is selected from the group consisting of GM-CSF, a GM-CSFR agonist antibody, and a small molecule activator of GM-CSFR. In some embodiments, the GM-CSFR activator is GM-CSF. In some embodiments, the plurality of the S/D/M factors further comprise an IL-6 receptor (IL-6R) activator, optionally wherein the IL-6R activator is selected from the group consisting of IL-6, an IL-6R agonist antibody, and a small molecule activator of IL-6R. In some embodiments, the IL-6R activator is IL-6.

### BRIEF DESCRIPTION OF THE DRAWINGS

FIGS. 1A-1E depict failure of monocytes from cancer patients (cMo) to respond to macrophage/DC-differentiation factors M-CSF and GM-CSF, which induce differentiation of monocytes from healthy donors (Mo). FIGS. 1A-1C show that high rates of cMo cell death were associated with failure of differentiation. FIG. 1C shows that combination of tumor-conditioned medium partially protected cMo from cell death while failed to induce differentiation by M-CSF plus GM-CSF. FIG. 1D shows cMo compared to Mo exhibit reduced MCSF-R and GMCSF-R expression. FIG. 1E shows that various reagent combinations failed to support cMo survival or differentiation.

FIGS. 2A-2C demonstrate the different signaling events occurring in Mo derived from healthy individuals versus cancer patients in response to M-CSF and to GM-CSF. FIG. 2A shows that M-CSF and GM-CSF: 1) triggered activation of Akt and Erk1/2 in Mo but not cMo, and 2) triggered caspase-9 and caspase-3 cleavage in cMo but not Mo, as demonstrated by increased phosphorylation of both Akt and Erk1/2 as assayed by Western blotting. FIG. 2B indicates the significant reduction in percentage of cMo cell survival compared to the percentage of Mo cell survival, as a result of apoptosis. FIG. 2C shows the total levels of common intracellular signaling proteins, including Akt and Erk1/2, which were not reduced in cMo cells compared to Mo.

FIGS. 3A-3I depict differentiation of monocytes from cancer patients (cMo) with TCR-activated T cells-conditioned medium (Karnelian X₁ or Carnelian X₁). FIG. 3A depicts the experimental scheme. FIGS. 3B-3C show the levels of cytokines produced from the first round of TCR stimulation of CD4 T cells derived from an individual with an autoimmune disorder, and the results demonstrated a high level of IL-10 production. FIG. 3D shows the cytokine profile of CD4 and CD8 T cells derived from an individual with an autoimmune disorder after the first and second round of TCR stimulation. FIGS. 3E and 3F show that morphology of cMo from patient SD-21-451 under treatment with activated T cell medium or M-CSF. FIG. 3F shows that the medium conditioned by TCR-activated T cells induces cMo differentiation. FIGS. 3G and 3H shows the morphology and cell surface expression of the antigen presentation machinery of cMo from patient SD-21-451 after differentiation by Karnelian X₁ for 72 hours, indicating that Karnelian X₁ differentiates cMo to professional APC. FIG. 3I indicates the percentage of cMo that survived and differentiated into κAPC, which was strongly supported using medium with high IL-10 concentration (i.e., the KX1 medium) but not in healthy CD4 Te cell medium lacking IL-10.

FIG. 4 depicts how monocytes from healthy donors or cancer patients survive and differentiate under different conditions (e.g., M-CSF, GM-CSF or Karnelian X₁).

FIGS. 5A-5K depicts important components in Karnelian X₁. FIG. 5A shows cytokines produced by T cells following the TCR stimulation. The medium collected on day 2 (post-stimulation 48 h) was termed Karnelian X₁. FIG. 5B shows that cytokine depletion assays identified IL-10, IFNγ, and TNFα in Karnelian X₁ to be the important for cMo survival. FIG. 5C shows that IL-10 (recombinant) dose-dependently restores IL-10-depleted Karnelian X₁ for supporting cMo survival. FIG. 5D shows the percent cMo survival when treated with the JAK inhibitor, C188-9, or STAT3 inhibitor, Napabucasin, in a dose-dependent reduction in cMo survival. FIG. 5E shows that STAT3 activators, Colivelin TFA and Garcinone D, both restored cMo survival in a dose-dependent manner for cMo cells grown in media with low IL-10 concentration. FIG. 5F depicts the percent of cMo survival for cells cultured with media containing an IL-10 family member cytokine (i.e., IL-19, IL-20, IL-22, and IL-24), which showed a similar dose-dependent response to treatment with IL-10 and thereby restored cMo survival. FIG. 5G depicts the percent of cMo survival for cells cultured with media containing an IL-12 family member cytokine (i.e., IL-12 and IL-23) following a similar pattern but at lower levels to treatment with IL-10, thereby restoring cMo survival. FIG. 5H shows that IL-12 treatment induced production of IL-10 by cMo. FIG. 5I provides a summary graphical representation of cMo survival and differentiation into κAPC in response to treatment with specific cytokines or STAT3 activators. FIG. 5J shows the percent of cMo survival when treated with IL-6 or IL-11 in low IL-10 media, which is significantly reduced compared to cMo cultured in media with IL-10. FIG. 5K shows the percent of cMo survival when treated with G-CSF in low IL-10 media, which is significantly reduced compared to cMo cultured in media with IL-10.

FIG. 6A-6G depicts the phenotypes of cMo grown in media supplemented with or lacking specific cytokines. FIG. 6A shows that cytokine depletion assays identified that IL-4, IFNγ, and TNFα are important for cMo phenotypic differentiation into APCs and depletion of IFNγ, TNFα, or IL-4 each delayed cMo-to-APC differentiation as indicated by reduced expression of MHC-II, CD80/86 and CD40. FIGS. 6B and 6D show that a cytokine cocktail (C-combo) recapitulated Karnelian X₁ for supporting cMo survival and APC differentiation.

FIG. 6C shows the impact of KX1 treatment on cMo derived from patients with different stages of liquid or solid cancers. FIGS. 6E-6G show a summary of components in Karnelian X₁ that support cMo-to-APC differentiation over 1 day and 2 days, respectively.

FIGS. 7A-7C depict mechanisms by which Carnelian X₁ induces cMo survival and differentiation into APCs. FIGS. 7A-7B shows cytokine receptor expression on cMo prior to and after Carnelian X₁ treatment compared to Mo from healthy donors. FIG. 7C depicts hypothesis that cytokine-mediated upregulation of IL-10R leads to cMo survival followed by GM-CSF, TNFα, IL-6 and IFNγ-mediated phenotypic differentiation to immunogenetic APCs.

FIGS. 8A-8C show that KX1 activated PI3K-Akt, MAPK, and STAT3 cell survival pathways and avoids caspase-mediated apoptosis. FIG. 8A shows that KX1 induced activation of Akt, Erk1/2, and STAT3 in cMo whereas M-CSF and GM-CSF both failed, as demonstrated by protein phosphorylation levels assessed by Western blotting. FIG. 8B demonstrates that KX1 treatment prevents cMo from undergoing apoptosis, as indicated by the levels of cleaved caspase-9 and caspase-3 measured by Western blotting. FIG. 8C shows the involvement of PI3K-Akt and MAPK pathways in KX1-mediated regulation of cMo survival such that inhibition of PI3K, Akt, or MAPK reduced cMo survival whereas inhibition of NFκB bore no impact on cMo survival.

FIGS. 9A-9D show that IL-10 induced survival signals in cMo. FIGS. 9A-9B shows cMo cultured with no treatment (NT), KX1, or KX1 supplemented with individual cytokine component depletion. Depletion of IL-10, IFNγ, and/or TNFα led to a reduction in cell survival and an increase in cleaved caspase-9 and caspase-3. FIG. 9C shows a time course activation of Akt, Erk1/2, and STAT3 as measured by protein phosphorylation levels in cMo grown in KX1 with IL-10 or depleted of IL-10. FIG. 9D shows the densitometry results calculated from the Western blotting images taken from FIG. 9C.

FIGS. 10A-10D show the C-combo component analyses. FIGS. 10A-10C depicted the C-combo V1 through V4 that were designed to test the minimal composition requirement for activation of Akt, Erk1/2, and STAT3 in cMo (FIGS. 10A-10B) and support cMo differentiation into κAPC (FIG. 10C). FIG. 10D compares KX1 media high in IL-10 to C-combo and found similar activation of Akt, Erk1/2, and STAT3 in cMo.

FIG. 11 depicts the phenotype of κAPC differentiated from cMo that were derived from cancer patients and compared the expression of antigen presentation machinery in cells grown in KX1 with IL-10 supplementation or in C-combo media.

FIGS. 12A-12H depict optimization of APC phenotypes by Karnelian reagents (FIGS. 12A-12B: Opt 1-4; FIGS. 12C-12D: Opt 1-5). FIG. 12A shows phenotypes of the APCs differentiated from cMo from cancer patients after treatment with a) Karnelian X₁ for 48 hours and b) Opt 1, 2, 3, or 4 for additional 24 hours. Karnelian X₂ is Opt 3 identified in FIG. 12B. FIG. 12C shows phenotypes of the APCs differentiated from cMo from cancer patients after treatment with a) Karnelian X₁ for 48 hours and b) Opt 1, 2, 3, 4, or 5 for additional 24 hours. FIG. 12D indicates that Karnelian X₂ is Opt 5. FIG. 12E depicts the determination of proinflammatory κAPC phenotype by detecting inflammatory cytokines secreted into the cell-free medium by κAPC. FIG. 12F depicts the determination of κAPC phagocytosis of tumor debris, wherein CFSE-stained tumor cells were snap frozen in liquid N2 followed by thawing on ice and adding the tumor cell debris to κAPC culture in KX2 Opt 5. FIG. 12G depicts the analyses of cell surface inhibitory receptors on κAPC that demonstrated low receptor levels except for the increased expression identified for LILRB1 and LILRB3. Inhibition of SHP-1 by TPI-1 was used to enhance the proinflammatory response. FIG. 12H depicts an example of the enhanced pro-inflammatory response to TPI-1 treatment.

FIGS. 13A-13B show the κAPC manufacturing protocol using KX1 and KX2. FIG. 13A depicts a summary of manufacturing κAPCs from cMo by Karnelian X₁ and X₂ reagents. Karnelian X₁ can be replaced by C-combo. FIG. 13B depicts surface expression of various molecules on fresh cMo and APCs derived from cMo after culture with Karnelian X₁ and Karnelian X₂.

FIG. 14A shows the cell morphology of κAPC assessed by light microscopy compared to immature DC, mature DC, M0 macrophages (MØ), M1 MØ, and M2 MØ using KX1 and/or KX2, and FIG. 14B shows the size of κAPC as compared to other types of cells.

FIGS. 15A-15B depicts the antigen presentation capability of cMo-derived κAPC and compares healthy Mo-derived κAPC to MoDC and M1 and M2 MØ. FIG. 15A shows a comparison of healthy Mo-derived κAPC to MoDC, M1 MØ, and M2 MØ derived from healthy individuals. FIG. 15B shows the levels of cMo-derived κAPC antigen presentation capability as assessed by flow cytometry, where the cMo were derived from patients with late-stage inoperable prostate cancer, colorectal cancer, or pancreatic cancer.

FIGS. 16A-16C show the gene signature from Nanostring transcriptional profiling of Mo, cMo, Mo-derived κAPC, and cMo-derived κAPC. FIG. 16A depicts the gene signature of Mo or cMo compared to LPS-treated mature MoDC, LPS- and IFNγ-treated MØ, and κAPC derived from healthy individuals and cancer patients. FIG. 16B shows the transcriptional analyses of Mo, cMo, Mo-derived κAPC, and cMo-derived κAPC and demonstrates that Mo and cMo share similar gene signatures, whereas some differences were identified in the Mo-derived κAPC and cMo-derived κAPC gene signatures. FIG. 16C provides the transcriptional analyses of cMo-derived κAPC grown in KX1 medium versus C-combo medium, wherein the gene signatures were similar.

FIG. 17 depicts a pair-comparison of gene transcription in 1) Mo-derived κAPC versus Mo, Mo-derived κAPC versus M1 MØ, and Mo-derived κAPC versus LPS-stimulated MoDC; 2) cMo-derived κAPC versus cMo and C-combo versus KX1 medium; and 3) Mo versus cMo and Mo-derived κAPC versus cMo-derived κAPC.

FIG. 18A-18B depicts the expression of different cell surface markers on κAPC compared to DC or MØ APCs. FIG. 18A depicts the cell surface markers found on different DC subtypes, on Mo-derived DC (MoDC), M1 MØ, and κAPC. FIG. 18B shows the flow cytometric analyses of the cell surface markers identified in FIG. 18A and demonstrates that κAPC do not share the same profile as cDC1, cDC2, or pDC or as MoDC or M1 MØ.

FIGS. 19A-19C provide an identification of κAPC-specific cell surface markers. FIG. 19A shows a comparison of cell surface expression between κAPC and MoDC and M1 MØ, where cell surface proteins were specifically increased on κAPC (i.e., IL-3R and CD32; indicated with an asterisk). FIG. 19B shows a comparison of cell surface proteins between κAPC and MoDC, M1 MØ, and M2 MØ, where Trem2, C3AR, IL-3R, LOX1, uPAR, CD40, and TLR2 were increased on κAPC compared to MoDC, M1 MØ, and M2 MØ. FIG. 19C shows the pattern recognition receptors expressed by κAPC compared to M1 MØ, wherein κAPC exhibited much higher levels of STING and TLR2.

FIG. 20 depicts a κAPC and tumor-focal RT combination therapy to KPC pancreatic ductal adenocarcinoma.

FIGS. 21A-21B depict therapeutic TT-κAPC vaccine against pancreatic cancer. Syngeneic KPC pancreatic ductal adenocarcinoma were established by subcutaneous engraftment. FIG. 21A depicts experimental scheme and tumor volume changes in mice without or with TT-κAPC vaccination via intratumoral (i.t.) injection. FIG. 21B shows that TME analyses reveal markedly increased CD45+ immune cells, especially the population of CD8 T cells, in TT-κAPC vaccinated tumor associated with tumor regression.

FIGS. 23A-23D depict NeoT cell targeting and clearance of multiple myeloma (MM) in vitro. FIG. 23A shows the degree of NeoT cell expansion after activation by κAPC or by anti-CD3/anti-CD28 antibody stimulation. FIG. 23B provides light microscopy images of NeoT expansion after co-culturing with MM antigen-loaded κAPC. FIG. 23C depicts the MM-specific NeoT cells exhibiting potent cytolytic activity against autologous MM cells in the total bone marrow, with avoiding non-marrow cells. NeoT activated by anti-CD3/anti-CD28 antibody stimulation showed limited cytolytic efficacy against MM cells. FIG. 23D provides still images from time-lapse videos of NeoT cells killing MM cells in in vitro co-cultures.

FIGS. 24A-24C depict the κAPC activation of NeoT cells derived from ovarian cancer tumor infiltrating lymphocytes (TILs). FIG. 24A shows NeoT cell proliferation after activation by κAPC or anti-CD3/anti-CD28 antibody stimulation, wherein the NeoT continuously respond to κAPC activation but fail to respond to a second round of anti-CD3/anti-CD28 antibody stimulation. FIG. 24B compares CD4 and CD8 T cell numbers in TILs before the addition of κAPC to the culture in vitro, followed by CD4 and CD8 T cell numbers after 10-day incubation with κAPC. After 10-day expansion, the Neo-Ts showed significant expansion in vitro. FIG. 24C provides light microscopy images of NeoT cells targeting dissociated ovarian cancer cells in vitro for cytolysis, which was recorded by time-lapse video. Graphical representation of the percent of ovarian cancer cells that were lysed showed a significant increase in tumor-specific cytotoxicity of κAPC-activated Neo-Ts.

FIG. 25 depicts the activation by κAPC of autologous NeoT cells that were derived from TILs of various solid tumors (i.e., liver metastatic urothelial carcinoma, ovarian cancer, colon cancer, kidney cancer, and lung cancer).

FIG. 26 depicts autologous NeoT cells engaging with κAPC and then activating and clonally expanding in vitro.

FIGS. 27A-27C depicts development of Neo-T therapy to a patient with metastatic uterine leiomyosarcoma. FIG. 27A shows differentiation of κAPC from autologous PBMC monocytes (cMo) using Karnelian X₁ and Karnelian X₂. The κAPC displayed potent antigen presentation capacity. FIG. 27B shows production of Neo-T in vitro. FIG. 27C shows Neo-T efficacy for killing cancer cells. A dose of ˜2×10⁷ Neo-Ts were intratumorally administrated (i.t.) into a liver metastatic mass (upper panel) and this treatment induced tumor volume reduction as assessed 4-weeks later (lower panel).

FIGS. 28A-28B depicts the tumor progression as measured by change in tumor tumor volume (FIG. 28A) and overall survival (FIG. 28B) of C57Bl/6 mice engrafted with KPC pancreatic ductal adenocarcinoma that are given NeoT adaptive cell therapy.

### DETAILED DESCRIPTION OF THE INVENTION

The present application provides novel compositions and agents that convert a large number of monocytes, particularly monocytes from cancer patients, into powerful antigen presenting cells (hereinafter referred to as “APCs”). These APCs can in turn be used to activate immune cells, rending them highly effective therapeutic agents for cancer treatment.

Inventors discovered that monocytes from cancer patients (referred to as “cMo” or “cMos”) are incapable of or inefficient of being differentiated to become dendritic cell- or macrophage-like APCs by traditional methods such as stimulation with M-CSF and/or GM-CSF. Such suppression surprisingly can be “unlocked” by cell culture supernatant of activated T cells that comprises a high level of IL-10. Inventors surprisingly discovered that IL-10, which is generally believed to be immunosuppressive in nature, is essential for removing the immunosuppression state of cMos. Without being bound by theory, it appears that IL-10, by activating IL-10 receptor, provides and differentiation factors (such as other cytokines). It was also found that IFNγ and TNFα present in the supernatant contribute to the survival of cancer monocytes, and that IL-4, IFNγ, and TNFα in the supernatant further promote differentiation from monocytes to APCs. Based on these profound findings, inventors created de novo compositions comprising one or more of these critical factors and demonstrated that IL-10 receptor activator (such as IL-10), along with one or more agents selected from the group consisting of an IL-4 receptor (IL-4R) activator, a TNFα receptor (TNFR) activator, and an interferon γ (IFNγ) receptor (IFNGR) activator, and demonstrated that such composition can achieve the same results initially observed with the supernatant. The APCs, when loaded with tumor associated peptides (tumor cells/antigens), were shown to conduct antigen presentation and activate tumor-antigen-specific CD4 and CD8 T cells, rendering them particularly effective in treating cancer.

Thus, the present application provides methods of generating APCs from monocytes (such as cMos), use of the APCs to activate immune cells, and use of the activated immune cells in treating cancer. Also provided are compositions comprising the critical factors discussed herein.

Accordingly, the present application in one aspect provides methods of stimulating a population of monocytes from an individual to produce a population of antigen presenting cells (“APCs”), comprising contacting the population of monocytes with a plurality of survival, differentiation and/or maturation factors (“S/D/M factors”) separately or simultaneously, wherein the plurality of S/D/M factors comprise: 1) an IL-10 receptor (IL-10R) activator and 2) one or more agents selected from the group consisting of: an IL-4 receptor (IL-4R) activator, a TNFα receptor (TNFR) activator, and an interferon γ (IFNγ) receptor (IFNGR) activator, thereby obtaining a population of APCs. The S/D/M factors need not perform the same function.

The present application in another aspect provides a population of APCs, such as APCs generated by some of the above methods and use of the APCs for cancer treatment.

The present application in another aspect provides method of activating a population of immune cells, comprising co-culturing the population of immune cells (e.g., T cells) with the population of the APCs described herein, wherein the APCs are pre-loaded with one or more neoantigen peptides. A population of activated immune cells obtained with the said methods, and methods of treating cancer by administering the activated immune cells are also provided.

The present application in another aspect provides compositions comprising a plurality of survival, differentiation and/or maturation factors (“S/D/M factors”), wherein the plurality of S/D/M factors comprise: 1) an IL-10 receptor (IL-10R) activator and 2) one or more agents selected from the group consisting of: an IL-4 receptor (IL-4R) activator, a TNFα receptor (TNFR) activator, and an interferon γ (IFNγ) receptor (IFNGR) activator.

### I. Definitions

In general, terms used in the claims and the specification are intended to be construed as having the plain meaning understood by a person of ordinary skill in the art. Certain terms are defined below to provide additional clarity. In case of conflict between the plain meaning and the provided definitions, the provided definitions are to be used.

The term “individual,” “subject,” or “patient” is used synonymously herein to describe a mammal, including humans. An individual includes, but is not limited to, human, bovine, horse, feline, canine, rodent, or primate. In some embodiments, the individual is human. In some embodiments, an individual suffers from a disease, such as cancer. In some embodiments, the individual is in need of treatment.

“Monocytes,” and “cells” as used herein, are understood to refer not only to the monocytes or cells when obtained, but to the progeny or potential progeny of such a cell. Because certain modifications may occur in succeeding generations due to e.g., environmental influences, such progeny may not, in fact, be identical to the parent cell, but are still included within the scope of the term as used herein.

“High level” or “higher level,” “low level,” or “lower level,” when referring to expression of a surface molecule in a population of the cells (e.g., monocytes or APCs) refers to how the average expression level of the particular surface molecule on the population of the cells as compared to the average level of the surface molecule on a reference cell population. Unless particularly stated otherwise, the reference cell population refers to a corresponding cell population derived from a healthy donor. In some cases, a high level of a particular molecule is defined when the expression level of the molecule on the recited cell population is at least about 20% (such as 20%, 30%, 40%, 50%, or more) higher than that on the reference cell population. In some cases, a low level of a particular molecule is defined when the expression level of the molecule on the recited cell population is at least about 20% (such as 20%, 30%, 40%, 50%, or more) lower than that on the reference cell population.

A “reference” as used herein, refers to any sample, standard, or level that is used for comparison purposes. A reference may be obtained from a healthy and/or non-diseased sample. In some examples, a reference may be obtained from an untreated sample. In some examples, a reference is obtained from a non-diseased or non-treated sample of an individual. In some examples, a reference is obtained from one or more healthy individuals who are not the individual or patient.

As used herein the term “antigen” is a substance that induces an immune response.

As used herein the term “neoantigen” is an antigen that has at least one alteration that makes it distinct from the corresponding wild-type, parental antigen, e.g., via mutation in a tumor cell or post-translational modification specific to a tumor cell. A neoantigen can include a polypeptide sequence. A mutation that results in a neoantigen can include a frameshift or non-frameshift indel, missense or nonsense substitution, splice site alteration, genomic rearrangement or gene fusion, or any genomic or expression alteration giving rise to a neoORF. A mutations can also include a splice variant. Post-translational modifications specific to a tumor cell can include aberrant phosphorylation. Post-translational modifications specific to a tumor cell can also include a proteasome-generated spliced antigen. See Liepe et al., A large fraction of HLA class I ligands are proteasome-generated spliced peptides; Science. 2016 Oct. 21; 354 (6310): 354-358.

As used herein the term “tumor neoantigen” or “cancer neoantigen” is a neoantigen present in a subject's tumor cell or tissue but not in the subject's corresponding normal cell or tissue.

The term “peptide” refers to a polymer of amino acids no more than 100 amino acids (including fragments of a protein), which may be linear or branched, comprise modified amino acids, and/or be interrupted by non-amino acids. The term also encompasses an amino acid polymer that has been modified naturally or by intervention, including, for example, disulfide bond formation, glycosylation, lipidation, acetylation, phosphorylation, or any other manipulation or modification. Also included within this term are, for example, polypeptides containing one or more analogs of an amino acid (including, for example, unnatural amino acids, etc.), as well as other modifications known in the art. The peptides described herein may be naturally-occurring, i.e., obtained or derived from a natural source (e.g., blood) or synthesized (e.g., chemically synthesized or by synthesized by recombinant DNA techniques).

As used herein the term “epitope” is the specific portion of an antigen typically bound by an antibody or T-cell receptor.

As used herein the term “immunogenic” is the ability to elicit an immune response, e.g., via T-cells, B cells, or both.

As used herein the term “HLA binding affinity” “MHC binding affinity” means affinity of binding between a specific antigen and a specific MHC allele.

As used herein the term “HLA type” is the complement of HLA gene alleles.

As used herein, “activated T cells” refer to a population of monoclonal (e.g., encoding the same TCR) or polyclonal (e.g., with clones encoding different TCRs) T cells that have T cell receptors that recognize at least one tumor antigen peptide. Activated T cells may contain one or more subtypes of T cells, including, but not limited to, cytotoxic T cells (e.g., CD8 T cells), helper T cells (e.g., CD4 T cells), natural killer T cells, γδ T cells, regulatory T cells, and memory T cells.

As used herein, “treatment” or “treating” is an approach for obtaining beneficial or desired results including clinical results. For purposes of this invention, beneficial or desired clinical results include, but are not limited to, one or more of the following: decreasing one more symptoms resulting from the disease, diminishing the extent of the disease, stabilizing the disease (e.g., preventing or delaying the worsening of the disease), preventing or delaying the spread (e.g., metastasis) of the disease, preventing or delaying the occurrence or recurrence of the disease, delay or slowing the progression of the disease, ameliorating the disease state, providing a remission (whether partial or total) of the disease, decreasing the dose of one or more other medications required to treat the disease, delaying the progression of the disease, increasing the quality of life, and/or prolonging survival. Also encompassed by “treatment” is a reduction of pathological consequence of cancer. The methods of the invention contemplate any one or more of these aspects of treatment.

As used herein, “delaying” the development of cancer means to defer, hinder, slow, retard, stabilize, and/or postpone development of the disease. This delay can be of varying lengths of time, depending on the history of the disease and/or individual being treated. As is evident to one skilled in the art, a sufficient or significant delay can, in effect, encompass prevention, in that the individual does not develop the disease. A method that “delays” development of cancer is a method that reduces probability of disease development in a given time frame and/or reduces the extent of the disease in a given time frame, when compared to not using the method. Such comparisons are typically based on clinical studies, using a statistically significant number of individuals. Cancer development can be detectable using standard methods, including, but not limited to, computerized axial tomography (CAT Scan), Magnetic Resonance Imaging (MRI), abdominal ultrasound, clotting tests, arteriography, or biopsy. Development may also refer to cancer progression that may be initially undetectable and includes occurrence, recurrence, and onset.

The term “simultaneous administration,” as used herein, means that a first therapy and second therapy in a combination therapy are administered with a time separation of no more than about 15 minutes, such as no more than about any of 10, 5, or 1 minutes. When the first and second therapies are administered simultaneously, the first and second therapies may be contained in the same composition (e.g., a composition comprising both a first and second therapy) or in separate compositions (e.g., a first therapy in one composition and a second therapy is contained in another composition).

As used herein, the term “sequential administration” means that the first therapy and second therapy in a combination therapy are administered with a time separation of more than about 15 minutes, such as more than about any of 20, 30, 40, 50, 60, or more minutes. Either the first therapy or the second therapy may be administered first. The first and second therapies are contained in separate compositions, which may be contained in the same or different packages or kits.

As used herein, the term “concurrent administration” means that the administration of the first therapy and that of a second therapy in a combination therapy overlap with each other.

As used herein, by “pharmaceutically acceptable” or “pharmacologically compatible” is meant a material that is not biologically or otherwise undesirable, e.g., the material may be incorporated into a pharmaceutical composition administered to an individual without causing any significant undesirable biological effects or interacting in a deleterious manner with any of the other components of the composition in which it is contained. Pharmaceutically acceptable carriers or excipients have preferably met the required standards of toxicological and manufacturing testing and/or are included on the Inactive Ingredient Guide prepared by the U.S. Food and Drug administration.

It is understood that embodiments of the application described herein include “consisting” and/or “consisting essentially of” embodiments.

Reference to “about” a value or parameter herein includes (and describes) variations that are directed to that value or parameter per se. For example, description referring to “about X” includes description of “X”.

As used herein, reference to “not” a value or parameter generally means and describes “other than” a value or parameter. For example, the method is not used to treat cancer of type X means the method is used to treat cancer of types other than X.

The term “about X-Y” used herein has the same meaning as “about X to about Y.”

It should be noted that, as used in the specification and the appended claims, the singular forms “a.” “an,” and “the” include plural referents unless the context clearly dictates otherwise.

Any terms not directly defined herein shall be understood to have the meanings commonly associated with them as understood within the art of the invention. Certain terms are discussed herein to provide additional guidance to the practitioner in describing the compositions, devices, methods and the like of aspects of the invention, and how to make or use them. It will be appreciated that the same thing may be said in more than one way. Consequently, alternative language and synonyms may be used for any one or more of the terms discussed herein. No significance is to be placed upon whether or not a term is elaborated or discussed herein. Some synonyms or substitutable methods, materials and the like are provided. Recital of one or a few synonyms or equivalents does not exclude use of other synonyms or equivalents, unless it is explicitly stated. Use of examples, including examples of terms, is for illustrative purposes only and does not limit the scope and meaning of the aspects of the invention herein.

### II. Method of Stimulating a Population of Monocytes to Generating APCs

The present application provides various methods of stimulating a population of monocytes from an individual to produce a population of APCs.

In some embodiments, there is provided a method of stimulating a population of monocytes from an individual (e.g., a cancer patient) to promote survival of the monocytes, comprising contacting the population of monocytes with an IL-10 receptor (IL-10R) activator (e.g., IL-10, e.g., IL-12).

In some embodiments, there is provided a method of stimulating a population of monocytes from an individual (e.g., a cancer patient) to produce a population of antigen presenting cells (“APCs”), comprising contacting the population of monocytes with a plurality of survival, differentiation and/or maturation factors (“S/D/M factors”) separately or simultaneously, wherein the plurality of S/D/M factors comprise: 1) an IL-10 receptor (IL-10R) activator (e.g., IL-10) and 2) one or more agents (e.g., two or more) selected from the group consisting of: an IL-4 receptor (IL-4R) activator (e.g., IL-4), a TNFα receptor (TNFR) activator (e.g., TNFα), and an interferon γ (IFNγ) receptor (IFNGR) activator (e.g., IFNγ), thereby obtaining a population of APCs. In some embodiments, there is provided a method of stimulating a population of monocytes from an individual (e.g., a cancer patient) to produce a population of antigen presenting cells (“APCs”), comprising contacting the population of monocytes with a plurality of survival, differentiation and/or maturation factors (“S/D/M factors”) separately or simultaneously, wherein the plurality of S/D/M factors comprise: 1) IL-10 and 2) two or more selected from the group consisting of: an IL-4 receptor (IL-4R) activator (e.g., IL-4), a TNFα receptor (TNFR) activator (e.g., TNFα), and an interferon γ (IFNγ) receptor (IFNGR) activator (e.g., IFNγ), thereby obtaining a population of APCs. In some embodiments, the plurality of S/D/M factors are present in a single composition. In some embodiments, at least one of the plurality of S/D/M factors is provided separately from one of other S/D/M factors in the plurality of S/D/M factors. In some embodiments, the plurality of the S/D/M factors further comprise a GM-CSF receptor (GM-CSFR) activator (e.g., GM-CSF). In some embodiments, the plurality of the S/D/M factors further comprise an IL-6 receptor (IL-6R) activator (e.g., IL-6). In some embodiments, the monocytes are cultured for about 1-3 days (e.g., 2-3 days) in the presence of at least one of the S/D/M factors. In some embodiments, the plurality of S/D/M factors are comprised in a composition derived from a medium (e.g., supernatant) derived from a culture of T cells after being treated with anti-CD3 and anti-CD28 antibodies. In some embodiments, the T cells are CD4 T cells. In some embodiments, the T cells are CD8 T cells. In some embodiments, the T cells are isolated from PBMC of the same individual or a different individual and have not been previously treated with anti-CD3 and/or anti-CD28 antibodies prior to the treatment. In some embodiments, the T cells are isolated from PBMC of the same individual or a different individual and have been previously treated with anti-CD3 and/or anti-CD28 antibodies prior to the treatment. In some embodiments, the medium is derived from the culture after the T cells are treated with anti-CD3 and anti-CD28 antibodies for about 1-3 days, optionally for about 2 days.

In some embodiments, there is provided a method of stimulating a population of monocytes from an individual (e.g., a cancer patient) to produce a population of antigen presenting cells (“APCs”), comprising contacting the population of monocytes with a plurality of survival, differentiation and/or maturation factors (“S/D/M factors”) separately or simultaneously, wherein the plurality of S/D/M factors comprise: 1) an IL-10 receptor (IL-10R) activator (e.g., IL-10), 2) an IL-4 receptor (IL-4R) activator (e.g., IL-4), 3) a TNFα receptor (TNFR) activator (e.g., TNFα), wherein the plurality of S/D/M factors are present in a single composition, thereby obtaining a population of APCs. In some embodiments, there is provided a method of stimulating a population of monocytes from an individual (e.g., a cancer patient) to produce a population of antigen presenting cells (“APCs”), comprising contacting the population of monocytes with a plurality of survival, differentiation and/or maturation factors (“S/D/M factors”) separately or simultaneously, wherein the plurality of S/D/M factors comprise: 1) IL-10, 2) IL-4, 3) TNFα, wherein the plurality of S/D/M factors are present in a single composition, thereby obtaining a population of APCs. In some embodiments, the IL-10 is a human IL-10 or a human recombination IL-10. In some embodiments, the IL-10 is present in the medium at a concentration of at least about 2 ng/ml, optionally at least about 10 ng/ml, further optionally about 10 ng/ml to about 200 ng/ml (e.g., about 20 ng/ml). In some embodiments, the TNFα is a human TNFα or a human recombinant TNFα. In some embodiments, the TNFα is present in the medium at a concentration of at least about 0.5 ng/ml, optionally at least about 1 ng/ml, further optionally about 0.5 ng/ml to about 30 ng/ml (e.g., about 1-10 ng/ml). In some embodiments, the IL-4 is a human IL-4 or a human recombinant IL-4. In some embodiments, the IL-4 is present in the medium at a concentration of at least about 15 μg/ml, optionally at least about 30 μg/ml, further optionally about 30 μg/ml to about 1 ng/ml (e.g., about 100 μg/ml to about 1 ng/ml). In some embodiments, the monocytes are cultured for about 1-3 days (e.g., 2-3 days) in the presence of at least one of the S/D/M factors. In some embodiments, the level of IL-10R on the monocytes before contacting the S/D/M factors is at least about 10%, 15%, 20%, 25%, 30%, 35%, 40%, 45% or 50% lower than the level of IL-10R on monocytes from a reference individual (e.g., a healthy individual). In some embodiments, the level of IL-4R on the monocytes before contacting the S/D/M factors is at least about 10%, 15%, 20%, 25%, 30%, 35%, 40%, 45% or 50% lower than the level of IL-4R on monocytes from a reference individual (e.g., a healthy individual).

In some embodiments, there is provided a method of stimulating a population of monocytes from an individual (e.g., a cancer patient) to produce a population of antigen presenting cells (“APCs”), comprising contacting the population of monocytes with a plurality of survival, differentiation and/or maturation factors (“S/D/M factors”) separately or simultaneously, wherein the plurality of S/D/M factors comprise: 1) an IL-10 receptor (IL-10R) activator (e.g., IL-10), 2) an IL-4 receptor (IL-4R) activator (e.g., IL-4), 3) a TNFα receptor (TNFR) activator (e.g., TNFα), wherein at least IL-4R activator is provided separated from the IL-10R activator or TNFR activator, thereby obtaining a population of APCs. In some embodiments, there is provided a method of stimulating a population of monocytes from an individual (e.g., a cancer patient) to produce a population of antigen presenting cells (“APCs”), comprising contacting the population of monocytes with a plurality of survival, differentiation and/or maturation factors (“S/D/M factors”) separately or simultaneously, wherein the plurality of S/D/M factors comprise: 1) IL-10, 2) IL-4, 3) TNFα, wherein at least IL-4 is provided separated from IL-10 or TNFα, thereby obtaining a population of APCs. In some embodiments, the IL-4R activator is provided after the IL-10R activator is provided. In some embodiments, the IL-4R activator is provided after the TNFR activator is provided. In some embodiments, the IL-10R activator and the TNFR activator are provided simultaneously. In some embodiments, the IL-10R activator and the TNFR activator are provided sequentially. In some embodiments, the IL-10 is a human IL-10 or a human recombination IL-10. In some embodiments, the IL-10 is present in the medium at a concentration of at least about 2 ng/ml, optionally at least about 10 ng/ml, further optionally about 10 ng/ml to about 200 ng/ml (e.g., about 20 ng/ml). In some embodiments, the TNFα is a human TNFα or a human recombinant TNFα. In some embodiments, the TNFα is present in the medium at a concentration of at least about 0.5 ng/ml, optionally at least about 1 ng/ml, further optionally about 0.5 ng/ml to about 30 ng/ml (e.g., about 1-10 ng/ml). In some embodiments, the IL-4 is a human IL-4 or a human recombinant IL-4. In some embodiments, the IL-4 is present in the medium at a concentration of at least about 15 μg/ml, optionally at least about 30 μg/ml, further optionally about 30 μg/ml to about 1 ng/ml (e.g., about 100 μg/ml to about 1 ng/ml). In some embodiments, the monocytes are cultured for about 1-3 days (e.g., 2-3 days) in the presence of at least one of the S/D/M factors. In some embodiments, the level of IL-10R on the monocytes before contacting the S/D/M factors is at least about 10%, 15%, 20%, 25%, 30%, 35%, 40%, 45% or 50% lower than the level of IL-10R on monocytes from a reference individual (e.g., a healthy individual). In some embodiments, the level of IL-4R on the monocytes before contacting the S/D/M factors is at least about 10%, 15%, 20%, 25%, 30%, 35%, 40%, 45% or 50% lower than the level of IL-4R on monocytes from a reference individual (e.g., a healthy individual).

In some embodiments, there is provided a method of stimulating a population of monocytes from an individual (e.g., a cancer patient) to produce a population of antigen presenting cells (“APCs”), comprising contacting the population of monocytes with a plurality of survival, differentiation and/or maturation factors (“S/D/M factors”) separately or simultaneously, wherein the plurality of S/D/M factors comprise: 1) an IL-10 receptor (IL-10R) activator (e.g., IL-10), 2) an IL-4 receptor (IL-4R) activator (e.g., IL-4), 3) an interferon γ (IFNγ) receptor (IFNGR) activator (e.g., IFNγ), wherein the plurality of S/D/M factors are present in a single composition, thereby obtaining a population of APCs. In some embodiments, there is provided a method of stimulating a population of monocytes from an individual (e.g., a cancer patient) to produce a population of antigen presenting cells (“APCs”), comprising contacting the population of monocytes with a plurality of survival, differentiation and/or maturation factors (“S/D/M factors”) separately or simultaneously, wherein the plurality of S/D/M factors comprise: 1) IL-10, 2) IL-4, 3) IFNγ, wherein the plurality of S/D/M factors are present in a single composition, thereby obtaining a population of APCs. In some embodiments, the IL-10 is a human IL-10 or a human recombination IL-10. In some embodiments, the IL-10 is present in the medium at a concentration of at least about 2 ng/ml, optionally at least about 10 ng/ml, further optionally about 10 ng/ml to about 200 ng/ml (e.g., about 20 ng/ml). In some embodiments, the IL-4 is a human IL-4 or a human recombinant IL-4. In some embodiments, the IL-4 is present in the medium at a concentration of at least about 15 μg/ml, optionally at least about 30 μg/ml, further optionally about 30 μg/ml to about 1 ng/ml (e.g., about 100 μg/ml to about 1 ng/ml). In some embodiments, the IFNγ is a human IFNγ or a human recombinant IFNγ. In some embodiments, the IFNγ is present in the medium at a concentration of at least about 5 ng/ml, optionally at least about 10 ng/ml, further optionally about 10 ng/ml to about 200 ng/ml (e.g., about 50-100 ng/ml). In some embodiments, the monocytes are cultured for about 1-3 days (e.g., 2-3 days) in the presence of at least one of the S/D/M factors. In some embodiments, the level of IL-10R on the monocytes before contacting the S/D/M factors is at least about 10%, 15%, 20%, 25%, 30%, 35%, 40%, 45% or 50% lower than the level of IL-10R on monocytes from a reference individual (e.g., a healthy individual). In some embodiments, the level of IL-4R on the monocytes before contacting the S/D/M factors is at least about 10%, 15%, 20%, 25%, 30%, 35%, 40%, 45% or 50% lower than the level of IL-4R on monocytes from a reference individual (e.g., a healthy individual).

In some embodiments, there is provided a method of stimulating a population of monocytes from an individual (e.g., a cancer patient) to produce a population of antigen presenting cells (“APCs”), comprising contacting the population of monocytes with a plurality of survival, differentiation and/or maturation factors (“S/D/M factors”) separately or simultaneously, wherein the plurality of S/D/M factors comprise: 1) an IL-10 receptor (IL-10R) activator (e.g., IL-10), 2) an IL-4 receptor (IL-4R) activator (e.g., IL-4), 3) an interferon γ (IFNγ) receptor (IFNGR) activator (e.g., IFNγ), wherein at least IL-4R activator is provided separated from the IL-10R activator or IFNGR activator, thereby obtaining a population of APCs. In some embodiments, there is provided a method of stimulating a population of monocytes from an individual (e.g., a cancer patient) to produce a population of antigen presenting cells (“APCs”), comprising contacting the population of monocytes with a plurality of survival, differentiation and/or maturation factors (“S/D/M factors”) separately or simultaneously, wherein the plurality of S/D/M factors comprise: 1) IL-10, 2) IL-4, 3) IFNγ, wherein at least IL-4 is provided separated from the IL-10 or IFNγ, thereby obtaining a population of APCs. In some embodiments, the IL-4R activator or IL-4 is provided after the IL-10R activator or IL-10 is provided. In some embodiments, the IL-4R activator or IL-4 is provided after the IFNGR activator or IFNγ is provided. In some embodiments, the IL-10R activator or IL-10 and the IFNGR activator or IFNγ are provided simultaneously. In some embodiments, the IL-10R activator or IL-10 and the IFNGR activator or IFNγ are provided sequentially. In some embodiments, the IL-10 is a human IL-10 or a human recombination IL-10. In some embodiments, the IL-10 is present in the medium at a concentration of at least about 2 ng/ml, optionally at least about 10 ng/ml, further optionally about 10 ng/ml to about 200 ng/ml (e.g., about 20 ng/ml). In some embodiments, the IL-4 is a human IL-4 or a human recombinant IL-4. In some embodiments, the IL-4 is present in the medium at a concentration of at least about 15 μg/ml, optionally at least about 30 μg/ml, further optionally about 30 μg/ml to about 1 ng/ml (e.g., about 100 μg/ml to about 1 ng/ml). In some embodiments, the IFNγ is a human IFNγ or a human recombinant IFNγ. In some embodiments, the IFNγ is present in the medium at a concentration of at least about 5 ng/ml, optionally at least about 10 ng/ml, further optionally about 10 ng/ml to about 200 ng/ml (e.g., about 50-100 ng/ml). In some embodiments, the monocytes are cultured for about 1-3 days (e.g., 2-3 days) in the presence of at least one of the S/D/M factors. In some embodiments, the level of IL-10R on the monocytes before contacting the S/D/M factors is at least about 10%, 15%, 20%, 25%, 30%, 35%, 40%, 45% or 50% lower than the level of IL-10R on monocytes from a reference individual (e.g., a healthy individual). In some embodiments, the level of IL-4R on the monocytes before contacting the S/D/M factors is at least about 10%, 15%, 20%, 25%, 30%, 35%, 40%, 45% or 50% lower than the level of IL-4R on monocytes from a reference individual (e.g., a healthy individual).

In some embodiments, there is provided a method of stimulating a population of monocytes from an individual (e.g., a cancer patient) to produce a population of antigen presenting cells (“APCs”), comprising contacting the population of monocytes with a plurality of survival, differentiation and/or maturation factors (“S/D/M factors”) separately or simultaneously, wherein the plurality of S/D/M factors comprise: 1) an IL-10 receptor (IL-10R) activator (e.g., IL-10), 2) a TNFα receptor (TNFR) activator (e.g., TNFα), and 3) an interferon γ (IFNγ) receptor (IFNGR) activator (e.g., IFNγ), wherein the plurality of S/D/M factors are present in a single composition, thereby obtaining a population of APCs. In some embodiments, there is provided a method of stimulating a population of monocytes from an individual (e.g., a cancer patient) to produce a population of antigen presenting cells (“APCs”), comprising contacting the population of monocytes with a plurality of survival, differentiation and/or maturation factors (“S/D/M factors”) separately or simultaneously, wherein the plurality of S/D/M factors comprise: 1) IL-10, 2) TNFα, and 3) IFNγ, wherein the plurality of S/D/M factors are present in a single composition, thereby obtaining a population of APCs. In some embodiments, the IL-10 is a human IL-10 or a human recombination IL-10. In some embodiments, the IL-10 is present in the medium at a concentration of at least about 2 ng/ml, optionally at least about 10 ng/ml, further optionally about 10 ng/ml to about 200 ng/ml (e.g., about 20 ng/ml). In some embodiments, the IFNγ is a human IFNγ or a human recombinant IFNγ. In some embodiments, the IFNγ is present in the medium at a concentration of at least about 5 ng/ml, optionally at least about 10 ng/ml, further optionally about 10 ng/ml to about 200 ng/ml (e.g., about 50-100 ng/ml). In some embodiments, the TNFα is a human TNFα or a human recombinant TNFα. In some embodiments, the TNFα is present in the medium at a concentration of at least about 0.5 ng/ml, optionally at least about 1 ng/ml, further optionally about 0.5 ng/ml to about 30 ng/ml (e.g., about 1-10 ng/ml). In some embodiments, the monocytes are cultured for about 1-3 days (e.g., 2-3 days) in the presence of at least one of the S/D/M factors. In some embodiments, the level of IL-10R on the monocytes before contacting the S/D/M factors is at least about 10%, 15%, 20%, 25%, 30%, 35%, 40%, 45% or 50% lower than the level of IL-10R on monocytes from a reference individual (e.g., a healthy individual). In some embodiments, the level of IL-4R on the monocytes before contacting the S/D/M factors is at least about 10%, 15%, 20%, 25%, 30%, 35%, 40%, 45% or 50% lower than the level of IL-4R on monocytes from a reference individual (e.g., a healthy individual).

In some embodiments, there is provided a method of stimulating a population of monocytes from an individual (e.g., a cancer patient) to produce a population of antigen presenting cells (“APCs”), comprising contacting the population of monocytes with a plurality of survival, differentiation and/or maturation factors (“S/D/M factors”) separately or simultaneously, wherein the plurality of S/D/M factors comprise: 1) an IL-10 receptor (IL-10R) activator (e.g., IL-10), 2) a TNFα receptor (TNFR) activator (e.g., TNFα), and 3) an interferon γ (IFNγ) receptor (IFNGR) activator (e.g., IFNγ), wherein at least IL-10R activator is provided separated from the TNFR activator or IFNGR activator, thereby obtaining a population of APCs. In some embodiments, there is provided a method of stimulating a population of monocytes from an individual (e.g., a cancer patient) to produce a population of antigen presenting cells (“APCs”), comprising contacting the population of monocytes with a plurality of survival, differentiation and/or maturation factors (“S/D/M factors”) separately or simultaneously, wherein the plurality of S/D/M factors comprise: 1) IL-10, 2) TNFα, and 3) IFNγ, wherein at least IL-10 is provided separated from the TNFα or IFNγ, thereby obtaining a population of APCs. In some embodiments, the IL-10R activator or IL-10 is provided before the TNFR activator or TNFα is provided. In some embodiments, the IL-10R activator or IL-10 is provided before the IFNGR activator or IFNγ is provided. In some embodiments, the TNFR activator or TNFα and the IFNGR activator or IFNγ are provided simultaneously. In some embodiments, the TNFR activator or TNFα and the IFNGR activator or IFNγ are provided sequentially. In some embodiments, the IL-10 is a human IL-10 or a human recombination IL-10. In some embodiments, the IL-10 is present in the medium at a concentration of at least about 2 ng/ml, optionally at least about 10 ng/ml, further optionally about 10 ng/ml to about 200 ng/ml (e.g., about 20 ng/ml). In some embodiments, the IFNγ is a human IFNγ or a human recombinant IFNγ. In some embodiments, the IFNγ is present in the medium at a concentration of at least about 5 ng/ml, optionally at least about 10 ng/ml, further optionally about 10 ng/ml to about 200 ng/ml (e.g., about 50-100 ng/ml). In some embodiments, the TNFα is a human TNFα or a human recombinant TNFα. In some embodiments, the TNFα is present in the medium at a concentration of at least about 0.5 ng/ml, optionally at least about 1 ng/ml, further optionally about 0.5 ng/ml to about 30 ng/ml (e.g., about 1-10 ng/ml). In some embodiments, the monocytes are cultured for about 1-3 days (e.g., 2-3 days) in the presence of at least one of the S/D/M factors. In some embodiments, the level of IL-10R on the monocytes before contacting the S/D/M factors is at least about 10%, 15%, 20%, 25%, 30%, 35%, 40%, 45% or 50% lower than the level of IL-10R on monocytes from a reference individual (e.g., a healthy individual). In some embodiments, the level of IL-4R on the monocytes before contacting the S/D/M factors is at least about 10%, 15%, 20%, 25%, 30%, 35%, 40%, 45% or 50% lower than the level of IL-4R on monocytes from a reference individual (e.g., a healthy individual).

In some embodiments, there is provided a method of stimulating a population of monocytes from an individual (e.g., a cancer patient) to produce a population of antigen presenting cells (“APCs”), comprising contacting the population of monocytes with a plurality of survival, differentiation and/or maturation factors (“S/D/M factors”) separately or simultaneously, wherein the plurality of S/D/M factors comprise: 1) an IL-10 receptor (IL-10R) activator (e.g., IL-10) and 2) an IL-4 receptor (IL-4R) activator (e.g., IL-4), 3) a TNFα receptor (TNFR) activator (e.g., TNFα), and 4) an interferon γ (IFNγ) receptor (IFNGR) activator (e.g., IFNγ), wherein the plurality of S/D/M factors are present in a single composition, thereby obtaining a population of APCs. In some embodiments, there is provided a method of stimulating a population of monocytes from an individual (e.g., a cancer patient) to produce a population of antigen presenting cells (“APCs”), comprising contacting the population of monocytes with a plurality of survival, differentiation and/or maturation factors (“S/D/M factors”) separately or simultaneously, wherein the plurality of S/D/M factors comprise: 1) IL-10, and 2) IL-4, 3) TNFα, and 4) IFNγ, wherein the plurality of S/D/M factors are present in a single composition, thereby obtaining a population of APCs. In some embodiments, the IL-10 is a human IL-10 or a human recombination IL-10. In some embodiments, the IL-10 is present in the medium at a concentration of at least about 2 ng/ml, optionally at least about 10 ng/ml, further optionally about 10 ng/ml to about 200 ng/ml (e.g., about 20 ng/ml). In some embodiments, the IFNγ is a human IFNγ or a human recombinant IFNγ. In some embodiments, the IFNγ is present in the medium at a concentration of at least about 5 ng/ml, optionally at least about 10 ng/ml, further optionally about 10 ng/ml to about 200 ng/ml (e.g., about 50-100 ng/ml). In some embodiments, the TNFα is a human TNFα or a human recombinant TNFα. In some embodiments, the TNFα is present in the medium at a concentration of at least about 0.5 ng/ml, optionally at least about 1 ng/ml, further optionally about 0.5 ng/ml to about 30 ng/ml (e.g., about 1-10 ng/ml). In some embodiments, the IL-4 is a human IL-4 or a human recombinant IL-4. In some embodiments, the IL-4 is present in the medium at a concentration of at least about 15 μg/ml, optionally at least about 30 μg/ml, further optionally about 30 μg/ml to about 1 ng/ml (e.g., about 100 μg/ml to about 1 ng/ml). In some embodiments, the monocytes are cultured for about 1-3 days (e.g., 2-3 days) in the presence of at least one of the S/D/M factors. In some embodiments, the level of IL-10R on the monocytes before contacting the S/D/M factors is at least about 10%, 15%, 20%, 25%, 30%, 35%, 40%, 45% or 50% lower than the level of IL-10R on monocytes from a reference individual (e.g., a healthy individual). In some embodiments, the level of IL-4R on the monocytes before contacting the S/D/M factors is at least about 10%, 15%, 20%, 25%, 30%, 35%, 40%, 45% or 50% lower than the level of IL-4R on monocytes from a reference individual (e.g., a healthy individual).

In some embodiments, there is provided a method of stimulating a population of monocytes from an individual (e.g., a cancer patient) to produce a population of antigen presenting cells (“APCs”), comprising contacting the population of monocytes with a plurality of survival, differentiation and/or maturation factors (“S/D/M factors”) separately or simultaneously, wherein the plurality of S/D/M factors comprise: 1) an IL-10 receptor (IL-10R) activator (e.g., IL-10) and 2) an IL-4 receptor (IL-4R) activator (e.g., IL-4), 3) a TNFα receptor (TNFR) activator (e.g., TNFα), and 4) an interferon γ (IFNγ) receptor (IFNGR) activator (e.g., IFNγ), wherein at least one of the plurality of S/D/M factors is provided separately from at least one of the other S/D/M factors in the plurality of S/D/M factors, thereby obtaining a population of APCs. In some embodiments, there is provided a method of stimulating a population of monocytes from an individual (e.g., a cancer patient) to produce a population of antigen presenting cells (“APCs”), comprising contacting the population of monocytes with a plurality of survival, differentiation and/or maturation factors (“S/D/M factors”) separately or simultaneously, wherein the plurality of S/D/M factors comprise: 1) IL-10 and 2) IL-4, 3) TNFα, and 4) IFNγ, wherein at least one of the plurality of S/D/M factors is provided separately from at least one of the other S/D/M factors in the plurality of S/D/M factors, thereby obtaining a population of APCs. In some embodiments, the IL-10R activator or IL-10 is provided before at least one of the other factors (e.g., the IL-4R activator or IL-4) is provided. In some embodiments, the IL-4R activator or IL-4 is provided after at least one of the other factors (e.g., the IL-10R activator or IL-10, the IFNGR activator or IFNγ, or the TNFGR activator or TNFα) is provided. In some embodiments, the IL-10R activator or IL-10, the TNFR activator or TNFα, and the IFNGR activator or TNFα are provided simultaneously. In some embodiments, the IL-10R activator or IL-10, the TNFR activator or TNFα, and the IFNGR activator or IFNγ are provided sequentially. In some embodiments, the IL-4R activator or IL-4, the TNFR activator or TNFα, and the IFNGR activator or IFNγ are provided simultaneously. In some embodiments, the IL-4R activator or IL-4, the TNFR activator or TNFα, and the IFNGR activator or IFNγ are provided sequentially. In some embodiments, the IL-10 is a human IL-10 or a human recombination IL-10. In some embodiments, the IL-10 is present in the medium at a concentration of at least about 2 ng/ml, optionally at least about 10 ng/ml, further optionally about 10 ng/ml to about 200 ng/ml (e.g., about 20 ng/ml). In some embodiments, the IFNγ is a human IFNγ or a human recombinant IFNγ. In some embodiments, the IFNγ is present in the medium at a concentration of at least about 5 ng/ml, optionally at least about 10 ng/ml, further optionally about 10 ng/ml to about 200 ng/ml (e.g., about 50-100 ng/ml). In some embodiments, the TNFα is a human TNFα or a human recombinant TNFα. In some embodiments, the TNFα is present in the medium at a concentration of at least about 0.5 ng/ml, optionally at least about 1 ng/ml, further optionally about 0.5 ng/ml to about 30 ng/ml (e.g., about 1-10 ng/ml). In some embodiments, the IL-4 is a human IL-4 or a human recombinant IL-4. In some embodiments, the IL-4 is present in the medium at a concentration of at least about 15 μg/ml, optionally at least about 30 μg/ml, further optionally about 30 μg/ml to about 1 ng/ml (e.g., about 100 μg/ml to about 1 ng/ml). In some embodiments, the monocytes are cultured for about 1-3 days (e.g., 2-3 days) in the presence of at least one of the S/D/M factors. In some embodiments, the level of IL-10R on the monocytes before contacting the S/D/M factors is at least about 10%, 15%, 20%, 25%, 30%, 35%, 40%, 45% or 50% lower than the level of IL-10R on monocytes from a reference individual (e.g., a healthy individual). In some embodiments, the level of IL-4R on the monocytes before contacting the S/D/M factors is at least about 10%, 15%, 20%, 25%, 30%, 35%, 40%, 45% or 50% lower than the level of IL-4R on monocytes from a reference individual (e.g., a healthy individual).

In some embodiments, there is provided a method of stimulating a population of monocytes from an individual (e.g., a cancer patient) to produce a population of antigen presenting cells (“APCs”), comprising contacting the population of monocytes with a plurality of survival, differentiation and/or maturation factors (“S/D/M factors”) separately or simultaneously, wherein the plurality of S/D/M factors comprise: 1) an IL-10 receptor (IL-10R) activator (e.g., IL-10) and 2) an IL-4 receptor (IL-4R) activator (e.g., IL-4), 3) a TNFα receptor (TNFR) activator (e.g., TNFα), 4) an interferon γ (IFNγ) receptor (IFNGR) activator (e.g., IFNγ), and 5) one or both of a GM-CSF receptor (GM-SCFR) activator (e.g., GM-CSF) and an IL-6 receptor (IL-6R) activator (e.g., IL-6), wherein the plurality of S/D/M factors are present in a single composition, thereby obtaining a population of APCs. In some embodiments, there is provided a method of stimulating a population of monocytes from an individual (e.g., a cancer patient) to produce a population of antigen presenting cells (“APCs”), comprising contacting the population of monocytes with a plurality of survival, differentiation and/or maturation factors (“S/D/M factors”) separately or simultaneously, wherein the plurality of S/D/M factors comprise: 1) IL-10 and 2) IL-4, 3) TNFα, 4) IFNγ, and 5) one or both of GM-CSF and IL-6, wherein the plurality of S/D/M factors are present in a single composition, thereby obtaining a population of APCs. In some embodiments, the IL-10 is a human IL-10 or a human recombination IL-10. In some embodiments, the IL-10 is present in the medium at a concentration of at least about 2 ng/ml, optionally at least about 10 ng/ml, further optionally about 10 ng/ml to about 200 ng/ml (e.g., about 20 ng/ml). In some embodiments, the IFNγ is a human IFNγ or a human recombinant IFNγ. In some embodiments, the IFNγ is present in the medium at a concentration of at least about 5 ng/ml, optionally at least about 10 ng/ml, further optionally about 10 ng/ml to about 200 ng/ml (e.g., about 50-100 ng/ml). In some embodiments, the TNFα is a human TNFα or a human recombinant TNFα. In some embodiments, the TNFα is present in the medium at a concentration of at least about 0.5 ng/ml, optionally at least about 1 ng/ml, further optionally about 0.5 ng/ml to about 30 ng/ml (e.g., about 1-10 ng/ml). In some embodiments, the IL-4 is a human IL-4 or a human recombinant IL-4. In some embodiments, the IL-4 is present in the medium at a concentration of at least about 15 μg/ml, optionally at least about 30 μg/ml, further optionally about 30 μg/ml to about 1 ng/ml (e.g., about 100 μg/ml to about 1 ng/ml). In some embodiments, the IL-6 is a human IL-6 or a human recombinant IL-6. In some embodiments, the IL-6 is present in the medium at a concentration of at least about 1 μg/ml, optionally at least about 5 μg/ml, further optionally about 5 μg/ml to about 100 μg/ml (e.g., about 10-50 pg/ml, e.g., about 30 μg/ml). In some embodiments, the GM-CSF is a human GM-CSF or a human recombinant GM-CSF. In some embodiments, the GM-CSF is present in the medium at a concentration of at least about 30 μg/ml, optionally at least about 50 μg/ml, further optionally about 100 μg/ml to about 1 ng/ml (e.g., about 100 μg/ml to about 500 μg/ml, e.g., about 300 μg/ml). In some embodiments, the monocytes are cultured for about 1-3 days (e.g., 2-3 days) in the presence of at least one of the S/D/M factors. In some embodiments, the level of IL-10R on the monocytes before contacting the S/D/M factors is at least about 10%, 15%, 20%, 25%, 30%, 35%, 40%, 45% or 50% lower than the level of IL-10R on monocytes from a reference individual (e.g., a healthy individual). In some embodiments, the level of IL-4R on the monocytes before contacting the S/D/M factors is at least about 10%, 15%, 20%, 25%, 30%, 35%, 40%, 45% or 50% lower than the level of IL-4R on monocytes from a reference individual (e.g., a healthy individual).

In some embodiments, there is provided a method of stimulating a population of monocytes from an individual (e.g., a cancer patient) to produce a population of antigen presenting cells (“APCs”), comprising contacting the population of monocytes with a plurality of survival, differentiation and/or maturation factors (“S/D/M factors”) separately or simultaneously, wherein the plurality of S/D/M factors comprise: 1) an IL-10 receptor (IL-10R) activator (e.g., IL-10), 2) an IL-4 receptor (IL-4R) activator (e.g., IL-4), 3) a TNFα receptor (TNFR) activator (e.g., TNFα), 4) an interferon γ (IFNγ) receptor (IFNGR) activator (e.g., IFNγ), and 5) one or both of a GM-CSF receptor (GM-SCFR) activator (e.g., GM-CSF) and an IL-6 receptor (IL-6R) activator (e.g., IL-6), wherein at least one of the plurality of S/D/M factors is provided separately from at least one of the other S/D/M factors in the plurality of S/D/M factors, thereby obtaining a population of APCs. In some embodiments, there is provided a method of stimulating a population of monocytes from an individual (e.g., a cancer patient) to produce a population of antigen presenting cells (“APCs”), comprising contacting the population of monocytes with a plurality of survival, differentiation and/or maturation factors (“S/D/M factors”) separately or simultaneously, wherein the plurality of S/D/M factors comprise: 1) IL-10, 2) IL-4, 3) TNFα, 4) IFNγ, and 5) one or both of GM-CSF and IL-6, wherein at least one of the plurality of S/D/M factors is provided separately from at least one of the other S/D/M factors in the plurality of S/D/M factors, thereby obtaining a population of APCs. In some embodiments, the IL-10R activator or IL-10, and/or the GM-CSFR activator or GM-CSF is provided before at least one of the other factors (e.g., the IL-4R activator or IL-4, e.g., the IL-6R activator or IL-6) is provided. In some embodiments, the IL-4R activator or IL-4, and/or the IL-6R activator or IL-6 is provided after at least one of the other factors (e.g., the IL-10R activator or IL-10, e.g., the GM-CSFR activator or GM-CSF) is provided. In some embodiments, the IL-10R activator or IL-10, the TNFR activator or TNFα, and the IFNGR activator or IFNγ are provided simultaneously. In some embodiments, the IL-10R activator or IL-10, the TNFR activator or TNFα, and the IFNGR activator or IFNγ are provided sequentially. In some embodiments, the IL-4R activator or IL-4, the TNFR activator or TNFα, and the IFNGR activator or IFNγ are provided simultaneously. In some embodiments, the IL-4R activator or IL-4, the TNFR activator or TNFα, and the IFNGR activator or IFNγ are provided sequentially. In some embodiments, the IL-10 is a human IL-10 or a human recombination IL-10. In some embodiments, the IL-10 is present in the medium at a concentration of at least about 2 ng/ml, optionally at least about 10 ng/ml, further optionally about 10 ng/ml to about 200 ng/ml (e.g., about 20 ng/ml). In some embodiments, the IFNγ is a human IFNγ or a human recombinant IFNγ. In some embodiments, the IFNγ is present in the medium at a concentration of at least about 5 ng/ml, optionally at least about 10 ng/ml, further optionally about 10 ng/ml to about 200 ng/ml (e.g., about 50-100 ng/ml). In some embodiments, the TNFα is a human TNFα or a human recombinant TNFα. In some embodiments, the TNFα is present in the medium at a concentration of at least about 0.5 ng/ml, optionally at least about 1 ng/ml, further optionally about 0.5 ng/ml to about 30 ng/ml (e.g., about 1-10 ng/ml). In some embodiments, the IL-4 is a human IL-4 or a human recombinant IL-4. In some embodiments, the IL-4 is present in the medium at a concentration of at least about 15 μg/ml, optionally at least about 30 μg/ml, further optionally about 30 μg/ml to about 1 ng/ml (e.g., about 100 μg/ml to about 1 ng/ml). In some embodiments, the IL-6 is a human IL-6 or a human recombinant IL-6. In some embodiments, the IL-6 is present in the medium at a concentration of at least about 1 μg/ml, optionally at least about 5 μg/ml, further optionally about 5 μg/ml to about 100 μg/ml (e.g., about 10-50 pg/ml, e.g., about 30 μg/ml). In some embodiments, the GM-CSF is a human GM-CSF or a human recombinant GM-CSF. In some embodiments, the GM-CSF is present in the medium at a concentration of at least about 30 μg/ml, optionally at least about 50 μg/ml, further optionally about 100 μg/ml to about 1 ng/ml (e.g., about 100 μg/ml to about 500 μg/ml, e.g., about 300 μg/ml). In some embodiments, the monocytes are cultured for about 1-3 days (e.g., 2-3 days) in the presence of at least one of the S/D/M factors. In some embodiments, the level of IL-10R on the monocytes before contacting the S/D/M factors is at least about 10%, 15%, 20%, 25%, 30%, 35%, 40%, 45% or 50% lower than the level of IL-10R on monocytes from a reference individual (e.g., a healthy individual). In some embodiments, the level of IL-4R on the monocytes before contacting the S/D/M factors is at least about 10%, 15%, 20%, 25%, 30%, 35%, 40%, 45% or 50% lower than the level of IL-4R on monocytes from a reference individual (e.g., a healthy individual).

In some embodiments, there is provided a method of stimulating a population of monocytes from an individual (e.g., a cancer patient) to produce a population of antigen presenting cells (“APCs”), comprising contacting the population of monocytes with a plurality of survival, differentiation and/or maturation factors (“S/D/M factors”) separately or simultaneously, wherein the plurality of S/D/M factors comprise: 1) IL-10 (e.g., a human IL-10 or a human recombinant IL-10) and 2) IL-4 (e.g., a human IL-4 or a human recombinant IL-4), 3) TNFα (e.g., a human TNFα or a human recombinant TNFα), 4) IFNγ (e.g., a human IFNγ or a human recombinant IFNγ), and 5) GM-CSF (e.g., a human GM-CSF or a human recombinant GM-CSF), and 6) IL-6 (e.g., a human IL-6 or a human recombinant IL-6), wherein the plurality of S/D/M factors are present in a single composition, thereby obtaining a population of APCs. In some embodiments, the IL-10 is a human IL-10 or a human recombination IL-10. In some embodiments, the IL-10 is present in the medium at a concentration of at least about 2 ng/ml, optionally at least about 10 ng/ml, further optionally about 10 ng/ml to about 200 ng/ml (e.g., about 20 ng/ml). In some embodiments, the IFNγ is a human IFNγ or a human recombinant IFNγ. In some embodiments, the IFNγ is present in the medium at a concentration of at least about 5 ng/ml, optionally at least about 10 ng/ml, further optionally about 10 ng/ml to about 200 ng/ml (e.g., about 50-100 ng/ml). In some embodiments, the TNFα is a human TNFα or a human recombinant TNFα. In some embodiments, the TNFα is present in the medium at a concentration of at least about 0.5 ng/ml, optionally at least about 1 ng/ml, further optionally about 0.5 ng/ml to about 30 ng/ml (e.g., about 1-10 ng/ml). In some embodiments, the IL-4 is a human IL-4 or a human recombinant IL-4. In some embodiments, the IL-4 is present in the medium at a concentration of at least about 15 μg/ml, optionally at least about 30 μg/ml, further optionally about 30 μg/ml to about 1 ng/ml (e.g., about 100 μg/ml to about 1 ng/ml). In some embodiments, the IL-6 is a human IL-6 or a human recombinant IL-6. In some embodiments, the IL-6 is present in the medium at a concentration of at least about 1 μg/ml, optionally at least about 5 μg/ml, further optionally about 5 μg/ml to about 100 μg/ml (e.g., about 10-50 pg/ml, e.g., about 30 μg/ml). In some embodiments, the GM-CSF is a human GM-CSF or a human recombinant GM-CSF. In some embodiments, the GM-CSF is present in the medium at a concentration of at least about 30 μg/ml, optionally at least about 50 μg/ml, further optionally about 100 μg/ml to about 1 ng/ml (e.g., about 100 μg/ml to about 500 μg/ml, e.g., about 300 μg/ml). In some embodiments, the monocytes are cultured for about 1-3 days (e.g., 2-3 days) in the presence of at least one of the S/D/M factors. In some embodiments, the level of IL-10R on the monocytes before contacting the S/D/M factors is at least about 10%, 15%, 20%, 25%, 30%, 35%, 40%, 45% or 50% lower than the level of IL-10R on monocytes from a reference individual (e.g., a healthy individual). In some embodiments, the level of IL-4R on the monocytes before contacting the S/D/M factors is at least about 10%, 15%, 20%, 25%, 30%, 35%, 40%, 45% or 50% lower than the level of IL-4R on monocytes from a reference individual (e.g., a healthy individual).

In some embodiments, there is provided a method of stimulating a population of monocytes from an individual (e.g., a cancer patient) to produce a population of antigen presenting cells (“APCs”), comprising contacting the population of monocytes with a plurality of survival, differentiation and/or maturation factors (“S/D/M factors”) separately or simultaneously, wherein the plurality of S/D/M factors comprise: 1) IL-10 (e.g., a human IL-10 or a human recombinant IL-10) and 2) IL-4 (e.g., a human IL-4 or a human recombinant IL-4), 3) TNFα (e.g., a human TNFα or a human recombinant TNFα), 4) IFNγ (e.g., a human IFNγ or a human recombinant IFNγ), and 5) GM-CSF (e.g., a human GM-CSF or a human recombinant GM-CSF), and 6) IL-6 (e.g., a human IL-6 or a human recombinant IL-6), wherein at least one of the plurality of S/D/M factors is provided separately from at least one of the other S/D/M factors in the plurality of S/D/M factors, thereby obtaining a population of APCs. In some embodiments, IL-10 and/or GM-CSF is provided before at least one of the other factors (e.g., IL-4 or IL-6) is provided. In some embodiments, IL-4 and/or IL-6 is provided after at least one of the other factors (e.g., IL-10 or GM-CSF) is provided. In some embodiments, IL-10, TNFα and IFNγ are provided simultaneously. In some embodiments, IL-10, TNFα and the IFNγ are provided sequentially. In some embodiments, IL-4, TNFα and the IFNγ are provided simultaneously. In some embodiments, IL-4, TNFα and the IFNγ are provided sequentially. In some embodiments, the IL-10 is a human IL-10 or a human recombination IL-10. In some embodiments, the IL-10 is present in the medium at a concentration of at least about 2 ng/ml, optionally at least about 10 ng/ml, further optionally about 10 ng/ml to about 200 ng/ml (e.g., about 20 ng/ml). In some embodiments, the IFNγ is a human IFNγ or a human recombinant IFNγ. In some embodiments, the IFNγ is present in the medium at a concentration of at least about 5 ng/ml, optionally at least about 10 ng/ml, further optionally about 10 ng/ml to about 200 ng/ml (e.g., about 50-100 ng/ml). In some embodiments, the TNFα is a human TNFα or a human recombinant TNFα. In some embodiments, the TNFα is present in the medium at a concentration of at least about 0.5 ng/ml, optionally at least about 1 ng/ml, further optionally about 0.5 ng/ml to about 30 ng/ml (e.g., about 1-10 ng/ml). In some embodiments, the IL-4 is a human IL-4 or a human recombinant IL-4. In some embodiments, the IL-4 is present in the medium at a concentration of at least about 15 μg/ml, optionally at least about 30 μg/ml, further optionally about 30 μg/ml to about 1 ng/ml (e.g., about 100 μg/ml to about 1 ng/ml). In some embodiments, the IL-6 is a human IL-6 or a human recombinant IL-6. In some embodiments, the IL-6 is present in the medium at a concentration of at least about 1 μg/ml, optionally at least about 5 μg/ml, further optionally about 5 μg/ml to about 100 μg/ml (e.g., about 10-50 pg/ml, e.g., about 30 μg/ml). In some embodiments, the GM-CSF is a human GM-CSF or a human recombinant GM-CSF. In some embodiments, the GM-CSF is present in the medium at a concentration of at least about 30 μg/ml, optionally at least about 50 μg/ml, further optionally about 100 μg/ml to about 1 ng/ml (e.g., about 100 μg/ml to about 500 μg/ml, e.g., about 300 μg/ml). In some embodiments, the monocytes are cultured for about 1-3 days (e.g., 2-3 days) in the presence of at least one of the S/D/M factors. In some embodiments, the level of IL-10R on the monocytes before contacting the S/D/M factors is at least about 10%, 15%, 20%, 25%, 30%, 35%, 40%, 45% or 50% lower than the level of IL-10R on monocytes from a reference individual (e.g., a healthy individual). In some embodiments, the level of IL-4R on the monocytes before contacting the S/D/M factors is at least about 10%, 15%, 20%, 25%, 30%, 35%, 40%, 45% or 50% lower than the level of IL-4R on monocytes from a reference individual (e.g., a healthy individual).

In some embodiments, there is provided a method of stimulating a population of monocytes from an individual having a cancer to produce a population of antigen presenting cells (“APCs”), comprising contacting the population of monocytes with a medium derived from a culture (e.g., a supernatant) of T cells after being treated with anti-CD3 and anti-CD28 antibodies, wherein the medium comprises an IL-10R activator (e.g., IL-10). In some embodiments, the medium further comprises an IL-4R activator (e.g., IL-4), an IFNGR activator (e.g., IFNγ), a TNFR activator (e.g., TNFα). In some embodiments, the medium further comprises a GM-CSFR activator (e.g., GM-CSF) and/or an IL-6R activator (e.g., IL-6). In some embodiments, the T cells are isolated from PBMC of the same individual or a different individual and have not been previously treated with anti-CD3 and/or anti-CD28 antibodies prior to the treatment. In some embodiments, the medium is derived from the culture after the T cells are treated with anti-CD3 and anti-CD28 antibodies for about 1-3 days, optionally for about 2 days. In some embodiments, the monocytes are cultured for about 2-3 days in the presence of the medium derived from the culture of T cells. In some embodiments, the IL-10 is a human IL-10 or a human recombination IL-10. In some embodiments, the IL-10 is present in the medium at a concentration of at least about 2 ng/ml, optionally at least about 10 ng/ml, further optionally about 10 ng/ml to about 200 ng/ml (e.g., about 20 ng/ml). In some embodiments, the IFNγ is a human IFNγ or a human recombinant IFNγ. In some embodiments, the IFNγ is present in the medium at a concentration of at least about 5 ng/ml, optionally at least about 10 ng/ml, further optionally about 10 ng/ml to about 200 ng/ml (e.g., about 50-100 ng/ml). In some embodiments, the TNFα is a human TNFα or a human recombinant TNFα. In some embodiments, the TNFα is present in the medium at a concentration of at least about 0.5 ng/ml, optionally at least about 1 ng/ml, further optionally about 0.5 ng/ml to about 30 ng/ml (e.g., about 1-10 ng/ml). In some embodiments, the IL-4 is a human IL-4 or a human recombinant IL-4. In some embodiments, the IL-4 is present in the medium at a concentration of at least about 15 μg/ml, optionally at least about 30 μg/ml, further optionally about 30 μg/ml to about 1 ng/ml (e.g., about 100 μg/ml to about 1 ng/ml). In some embodiments, the IL-6 is a human IL-6 or a human recombinant IL-6. In some embodiments, the IL-6 is present in the medium at a concentration of at least about 1 μg/ml, optionally at least about 5 μg/ml, further optionally about 5 μg/ml to about 100 μg/ml (e.g., about 10-50 pg/ml, e.g., about 30 μg/ml). In some embodiments, the GM-CSF is a human GM-CSF or a human recombinant GM-CSF. In some embodiments, the GM-CSF is present in the medium at a concentration of at least about 30 μg/ml, optionally at least about 50 μg/ml, further optionally about 100 μg/ml to about 1 ng/ml (e.g., about 100 μg/ml to about 500 μg/ml, e.g., about 300 μg/ml). In some embodiments, the monocytes are cultured for about 1-3 days (e.g., 2-3 days) in the presence of at least one of the S/D/M factors. In some embodiments, the level of IL-10R on the monocytes before contacting the S/D/M factors is at least about 10%, 15%, 20%, 25%, 30%, 35%, 40%, 45% or 50% lower than the level of IL-10R on monocytes from a reference individual (e.g., a healthy individual). In some embodiments, the level of IL-4R on the monocytes before contacting the S/D/M factors is at least about 10%, 15%, 20%, 25%, 30%, 35%, 40%, 45% or 50% lower than the level of IL-4R on monocytes from a reference individual (e.g., a healthy individual).

In some embodiments according to any of the embodiments described above, the method further comprises contacting the population of monocytes with a plurality of refinement factors selected from the group consisting of type-I interferon, IFNγ, TNFα, a TLR ligand, CD40L or a CD40-ligating antibody, an anti-PD-L1 antibody, and TPI-1, optionally wherein the type-I interferon comprises IFNα and/or IFNβ, and optionally wherein the TLR ligand is poly IC, CpG, or LPS. In some embodiments, the plurality of refinement factors are provided after the plurality of monocytes are contacted with the plurality of S/D/M factors or the medium derived from the culture of T cells, thereby producing the population of APCs, and wherein the population of APCs are cultured for about 1-5 days in the presence of the plurality of the refinement factors, optionally wherein the population of APCs are cultured for about one day. In some embodiments, the plurality of refinement factors are provided when a) at least about 50% of the monocytes survive, b) at least about 30% of the population of APCs exhibit a dendritic cell morphology and/or c) the population of APCs express i) a high level of one or more molecules selected from the group consisting of MHC I, MHC II, CD80, CD86, and/or CD40, and/or ii) a low level of SIRPα.

In some embodiments, the method further comprises contacting the population of monocytes with a plurality of refinement factors comprising IFNα, IFNγ, and TNFα.

In some embodiments, the refinement factors comprise IFNα, IFNγ, TNFα, poly IC, CpG.

In some embodiments, the refinement factors comprise IFNα, IFNγ, TNFα, poly IC, CpG, CD40L, and an anti-PD-L1 antibody.

In some embodiments, the refinement factors comprise IFNα, IFNγ, TNFα, poly IC. CpG, CD40L, TPI-1, and an anti-PD-L1 antibody.

Survival, Differentiation and/or Maturation Factors (“S/D/M Factors”)

In some embodiments, the plurality of survival, differentiation and/or maturation factors (“S/D/M factors”) described herein comprise: 1) an IL-10 receptor (IL-10R) activator (e.g., IL-10) and 2) one or more agents selected from the group consisting of: an IL-4 receptor (IL-4R) activator (e.g., IL-4), a TNFα receptor (TNFR) activator (e.g., TNFα), and an interferon γ (IFNγ) receptor (IFNGR) activator (e.g., IFNγ).

In some embodiments, the plurality of survival, differentiation and/or maturation factors (“S/D/M factors”) described herein comprise: 1) an IL-10 receptor (IL-10R) activator (e.g., IL-10) and 2) IFNγ. In some embodiments, the plurality of survival, differentiation and/or maturation factors (“S/D/M factors”) described herein comprise: 1) an IL-10 receptor (IL-10R) activator (e.g., IL-10) and 2) TNFα. In some embodiments, the plurality of survival, differentiation and/or maturation factors (“S/D/M factors”) described herein comprise: 1) an IL-10 receptor (IL-10R) activator (e.g., IL-10) and 2) IL-6. In some embodiments, the plurality of survival, differentiation and/or maturation factors (“S/D/M factors”) described herein comprise: 1) an IL-10 receptor (IL-10R) activator (e.g., IL-10) and 2) IL-4. In some embodiments, the plurality of survival, differentiation and/or maturation factors (“S/D/M factors”) described herein comprise: 1) an IL-10 receptor (IL-10R) activator (e.g., IL-10) and 2) GM-CSF. In some embodiments, the plurality of survival, differentiation and/or maturation factors (“S/D/M factors”) described herein comprise: 1) an IL-10 receptor (IL-10R) activator (e.g., IL-10) and 2) IL-12. In some embodiments, the plurality of survival, differentiation and/or maturation factors (“S/D/M factors”) described herein comprise: 1) an IL-10 receptor (IL-10R) activator (e.g., IL-10) and 2) poly: IC. In some embodiments, the plurality of survival, differentiation and/or maturation factors (“S/D/M factors”) described herein comprise: 1) an IL-10 receptor (IL-10R) activator (e.g., IL-10) and 2) CpG.

In some embodiments, the plurality of survival, differentiation and/or maturation factors (“S/D/M factors”) described herein comprise: 1) an IL-10 receptor (IL-10R) activator (e.g., IL-10), 2) a TNFα receptor (TNFR) activator (e.g., TNFα), and 3) an interferon γ (IFNγ) receptor (IFNGR) activator (e.g., IFNγ).

In some embodiments, the plurality of survival, differentiation and/or maturation factors (“S/D/M factors”) described herein comprise: 1) an IL-10 receptor (IL-10R) activator (e.g., IL-10), 2) a TNFα receptor (TNFR) activator (e.g., TNFα), 3) an interferon γ (IFNγ) receptor (IFNGR) activator (e.g., IFNγ), 4) an IL-6 receptor (IL-6R) activator (e.g., IL-6), and 5) a GM-CSF receptor (GM-CSF) activator (e.g., GM-CSF).

In some embodiments, the plurality of survival, differentiation and/or maturation factors (“S/D/M factors”) described herein comprise: 1) an IL-22, 2) a TNFα receptor (TNFR) activator (e.g., TNFα), 3) an interferon γ (IFNγ) receptor (IFNGR) activator (e.g., IFNγ), 4) an IL-6 receptor (IL-6R) activator (e.g., IL-6), and 5) a GM-CSF receptor (GM-CSF) activator (e.g., GM-CSF).

In some embodiments, the plurality of survival, differentiation and/or maturation factors (“S/D/M factors”) described herein comprise: 1) a TNFα receptor (TNFR) activator (e.g., TNFα), 2) an interferon γ (IFNγ) receptor (IFNGR) activator (e.g., IFNγ), 3) an IL-6 receptor (IL-6R) activator (e.g., IL-6), and 4) a GM-CSF receptor (GM-CSF) activator (e.g., GM-CSF).

In some embodiments, the plurality of survival, differentiation and/or maturation factors (“S/D/M factors”) described herein comprise: 1) an IL-10 receptor (IL-10R) activator (e.g., IL-10), 2) a TNFα receptor (TNFR) activator (e.g., TNFα), 3) an interferon γ (IFNγ) receptor (IFNGR) activator (e.g., IFNγ), 4) an IL-6 receptor (IL-6R) activator (e.g., IL-6), 5) an IL-4 receptor (IL-4R) activator (e.g., IL-4), and 6) a GM-CSF receptor (GM-CSF) activator (e.g., GM-CSF).

### IL-10 Receptor (IL-10R) Activator and IL-10

“IL-10 receptor (IL-10R) activator” described herein refers to a molecule that activates IL-10 receptor mediated signaling pathway. IL-10R includes both IL-10R1 and IL-10R2.

Interleukin 10 (IL-10), also known as human cytokine synthesis inhibitory factor (CSIF), is an anti-inflammatory cytokine. In humans, interleukin 10 is encoded by the IL10 gene. IL-10 signals through a receptor complex consisting of two IL-10 receptor-1 and two IL-10 receptor-2 proteins. Consequently, the functional receptor consists of four IL-10 receptor molecules. IL-10 binding induces STAT3 signaling via the phosphorylation of the cytoplasmic tails of IL-10 receptor 1 and IL-10 receptor 2 by JAK1 and Tyk2 respectively. See e.g., Saraiva, M., O'Garra, A. The regulation of IL-10 production by immune cells. Nat Rev Immunol 10, 170-181 (2010).

In humans, IL-10 is encoded by the IL10 gene, which is located on chromosome 1 and comprises 5 exons, and is primarily produced by monocytes and, to a lesser extent, lymphocytes, namely type-II T helper cells (TH2), mast cells, CD4+CD25+Foxp3+ regulatory T cells, and in a certain subset of activated T cells and B cells. IL-10 can be produced by monocytes upon PD-1 triggering in these cells.

IL-10 is a cytokine with multiple, pleiotropic, effects in immunoregulation and inflammation. It downregulates the expression of Th1 cytokines, MHC class II antigens, and co-stimulatory molecules on macrophages. It also enhances B cell survival, proliferation, and antibody production. IL-10 can block NF-κB activity and is involved in the regulation of the JAK-STAT signaling pathway.

IL-10 was initially reported to suppress cytokine secretion, antigen presentation, and CD4+ T cell activation. Further investigation has shown that IL-10 predominantly inhibits lipopolysaccharide (LPS) and bacterial product mediated induction of the pro-inflammatory cytokines TNFα, IL-1B, IL-12, and IFNγ secretion from Toll-Like Receptor (TLR) triggered myeloid lineage cells.

IL-10 referred herein comprises any constructs that have a component of IL-10 (e.g., a naturally occurring IL-10, e.g., a recombinant IL-10). These include and are not limited to natural IL-10 (e.g., various isoforms of human IL-10), synthetic or recombinant IL-10, and fusion proteins having an IL-10 component.

In some embodiments, the IL-10R activator further comprises a moiety that enhances stability or half-life, including for example an Fc portion or a PEG moiety.

In some embodiments, the IL-10R activator is selected from the group consisting of: an IL-10 (e.g., a pegylated IL-10, e.g., pegilodecakin or AM0010), an IL-10 family member (e.g., IL-19, IL-20, IL-22, IL-24, IL-26, IL-28), an IL-10R agonist antibody, a small molecule activator of IL-10R, and an activator of the IL-10R downstream STAT3 (e.g., Long noncoding RNA (LncRNA) PVT1, NEAT1, FEZF1-AS1, UICC). In some embodiments, the IL-10R activator is IL-10. In some embodiments, the IL-10 is a human IL-10 or a human recombinant IL-10. In some embodiments, the IL-10 (e.g., a human IL-10) is present in the medium at a concentration of at least about 2 ng/ml, optionally at least about 10 ng/ml (e.g., about 20 ng/ml). In some embodiments, the IL-10 (e.g., a human IL-10) is present in the medium at a concentration of about 2 ng/ml to about 200 ng/ml (e.g., about 10 ng/ml to about 200 ng/ml, e.g., about 10 ng/ml to about 100 ng/ml, e.g., about 20 ng/ml to about 100 ng/ml).

### STAT3 Activator and STAT3

In some embodiments, the IL-6R activator is a STAT3 activator. “STAT3 activator” described herein refers to a molecule that activates STAT3 signaling, e.g., the STAT3 nuclear localization and transcription factor activity.

STAT3 is a transcription factor that resides in the cytoplasm in its inactive, unphosphorylated form and translocates to the nucleus upon its activation via phosphorylation, e.g., Tyr705 phosphorylation, and subsequent dimerization. Upon entering the nucleus, the activated STAT3 dimer binds to the IFN-γ-activated sequence (GAS) in target promoters and thereby activates transcription of target genes. Multiple tyrosine kinases have been described as intracellular activators of STAT3 activity (e.g., JAK1, JAK2, EGFR, Src, and ERK). Further mechanisms of activation include: i) STAT3 phosphorylation at Ser727 by protein kinase C (PKC), mitogen-activated protein kinases (MAPKs), and CDK5; and ii) STAT3 acetylation on Lys685 by histone acetyltransferase, which can enhance STAT3 dimer stability. See, e.g., Rébé et al., JAKSTAT 2013; 2 (1): €23010.

STAT3 is expressed in most cell types under specific conditions, and generally is described to be involved with biological processes such as: cell proliferation, differentiation, apoptosis, angiogenesis, metastasis, inflammation, and immunity. In immune cells, STAT3 has been described in contradictory terms. For example, STAT3 has been described as promoting the differentiation of macrophages toward the M2 phenotype and the absence of functional dendritic cells (see, e.g., Rébé et al., 2013). STAT3 has also been described to promote the gp130-mediated maintenance of the pluripotential state of proliferating embryonic stem cells and for the gp130-induced macrophage differentiation of M1 cells. Both c-myc and pim have been identified as target genes of STAT3 and together can compensate for STAT3 in cell survival and cell-cycle transition (see, e.g., Hirano et al. Oncogene 2000; 19:2548-2556).

STAT3 activation is rapid and transient under normal biological conditions and mediated by many extracellular stimuli, including cytokines (IL-6, IL-10, IFNs, TNFα, LIF, OSM, etc.) and growth factors (e.g., EGF, G-CSF, GM-CSF, VEGF, HGF, GH, and Her2/Neu). Active oncogenic proteins, such as Src (e.g., v-Src) and Ras, as well as chemical carcinogens and other molecules also can activate STAT3. Indeed, many regulated genes induced by STAT3 activity in turn activate the same STAT3 pathways and thereby keep a stable feedforward loop. In some embodiments, the STAT3 activator comprises a cytokine selected from the group consisting of: IL-6, IL-10, IL-11, IL-12, IL-19, IL-20, IL-22, IL-23, IL-24, IL-26, IL-27, IFNα, IFNβ, IFNγ, TNFα, leukemia inhibitory factor (LIF), oncostatin M (OSM), biologically active derivatives thereof, and any combination thereof. In some embodiments, the STAT3 activator comprises a growth factor selected from the group consisting of: EGF, FGF, IGF, G-CSF, GM-CSF, VEGF, HGF, GF, Her2/Neu, biologically active derivatives thereof, and any combination thereof. In some embodiments, the STAT3 activator comprises a JAK activator, such as an enzyme that phosphorylates JAK (e.g., JAK2). In some embodiments, the STAT3 activator comprises a hormone (e.g., leptin). In some embodiments, the STAT3 activator comprises a chaperone protein (e.g., HSP90, HSP70, HSP27, HSP110, HOP).

STAT3 activity can be positively regulated by the signaling pathways of IL-10 and IL-10 family members, including IL-19, IL-20, IL-22, IL-24, and IL-26. Further, IL-12 and affiliated family members (e.g., IL-23) can activate STAT3 activity, at least in part by promoting IL-10/IL-10R production and autocrine signaling in the κAPC cells. IL-6 has also been described as an activator of the STAT3 pathway. In some embodiments, the STAT3 activator is selected from the group consisting of: IL-6, IL-10, IL-12, IL-19, IL-20, IL-22, IL-23, IL-24, IL-26, biologically active derivatives thereof, and any combination thereof. In some embodiments, the STAT3 activator is an IL-10R activator, such as any described herein or known in the art.

In some embodiments, the IL-10R activator is an activator of the IL-10R downstream STAT3. STAT3 activators can include, but are not limited to, any of: a small molecule, a nucleic acid (e.g., an siRNA, an shRNA, an antisense RNA, a microRNA), a nucleic acid base inhibitor (e.g., a circular RNA inhibitor), a nucleic acid editing system (e.g., CRISPR, ZEN, or TALENS systems), a decoy oligonucleotide, a peptide agent, a protein agent (e.g., an antibody agent that targets IL-10R; e.g., a protein agent that targets STAT3 phosphorylation and/or prevents STAT3 dephosphorylation), a protein stabilizing agent (e.g., a STAT3-stabilizing agent such as a chaperone protein, for example HSP90, HSP70, HSP27, HSP110, and/or HOP), a protein degrading or destabilizing agent (e.g., a phosphatase degrading or destabilizing agent such as a phosphatase-targeting PROTAC, LYTAC, molecular glue, AbTAC, CMATAC, etc.), a protein modified with an unnatural amino acid, a viral agent (e.g., Kaposi sarcoma herpesvirus (KSHV)), derivatives thereof, and any combination thereof.

In some embodiments, the STAT3 activator comprises a cancer cell STAT3 activator. These cancer cell STAT3 activators can include any of: PVT1, NEAT1, FEZF1-AS1, UICC, MALAT1, XIST, miR-30d, CD109, CD146, CD24, CDK7, SOX, Smad6, Smad7, TRIM24, TRIM27, TRIM59, ADAM12, USP22, BMX AKR1C1, PRMT1, PBX1, HSP110, RanBP6, RAC1-GTP, PA28γ, E6, and FABP5.

In some embodiments, the STAT3 activator comprises a small molecule selected from the group consisting of: Colivelin, Colivelin TFA, Garcinone D, Butyzamide, Eflepedocokin alfa, Broussonin E, derivatives thereof, and any combinations thereof. In some embodiments, the STAT3 activator comprises Colivelin, Colivelin TFA, and/or Garcinone D.

In some embodiments, the STAT3 activator comprises an inhibitor or antagonist of a molecule or compound that inactivates STAT3 (e.g., reduces phosphorylated STAT3 levels) or that reduces total STAT3 levels (e.g., via protcasomal degradation and/or transcriptional suppression) in a cell of interest, e.g., in a myeloid cell such as a κAPC cell. For example, molecules or compounds that can inactivate STAT3 include, but are not limited to: β-elemene, selective serotonin-reuptake inhibitors (SSRIs, e.g., fluoxetine), minecoside, Lutcolin (3,4,5,7-tetrahydroxyflavonc), SHP-1, SHP-2, PTP1B, PTPRM, cEF2 kinase, PKM2, curcumin, cucurbitacin, honokiol, guggulsterone, resveratrol, berbamine, flavopiridol, JAK inhibitors/inactivated JAK (e.g., JAK2), low molecular weight-DSP2, PIAS3, etc. In some embodiments, molecules or compounds that can reduce total STAT3 levels (e.g., via proteasomal degradation and/or transcriptional suppression) include, but are not limited to: PDLIM2, COP1, calcineurin, SOCS proteins, Rubulavirus (e.g., Mumps virus, e.g., the Mumps viral V protein, for example the V-dependent degradation complex VDC or V/DDB 1/Cullin degradation complex), TSM-1, KYM-003, KTX-201, SD-36, AUY922, 17-DMAG, etc. In some embodiments, the inhibitor of a molecule or compound that inactivates STAT3 or that reduces total STAT3 levels in a cell of interest competitively bind to STAT3 to prevent inactivation (e.g., DDIAS) or protein degradation (e.g., chaperones, such as HSP90).

See, e.g., Kim et al. Oncol Lett. 2022; 23 (3): 94; Zheng et al. Exp Mol Med. 2018; 50 (9): 1-14; Liao et al. Anticancer Res. 2022; 42 (8): 3807-3814; Xiao et al. Cell Commun Signal. 2020; 18 (1): 25; Jego et al. Cancers (Basel) 2020; 12 (1): 21; Liu et al. Cell Death Dis. 2014; 5 (6): c1293; and Yang et al. Cytokine Growth Factor Rev. 2019; 46:10-22. The amount of exemplified STAT3 activator can be seen in e.g., Table 1.

### IL-4 Receptor (IL-4R) Activator and IL-4

“IL-4 receptor (IL-4R) activator” described herein refers to a molecule that activates IL-4 receptor mediated signaling pathway.

Interleukin 4 (IL-4) is a cytokine that induces differentiation of naive helper T cells (Th0 cells) to Th2 cells. Upon activation by IL-4, Th2 cells subsequently produce additional IL-4 in a positive feedback loop. IL-4 is produced primarily by mast cells, Th2 cells, cosinophils and basophils. It is closely related and has functions similar to IL-13. Interleukin 4 has many biological roles, including the stimulation of activated B cell and T cell proliferation, and the differentiation of B cells into plasma cells. It is a key regulator in humoral and adaptive immunity. IL-4 induces B cell class switching to IgE, and up-regulates MHC class II production. IL-4 decreases the production of Th1 cells, macrophages, IFNγ, and dendritic cells IL-12. IL-4 signaling determines the levels of CD20 on the surface of normal and malignant B lymphocytes via activation of transcription factor STAT6. Overproduction of IL-4 is associated with allergies.

IL-4 referred herein comprises any constructs that have a component of IL-4 (e.g., a naturally occurring IL-4, e.g., a recombinant IL-4). These include and are not limited to natural IL-4 (e.g., various isoforms of human IL-4), synthetic or recombinant IL-4, and fusion proteins having an IL-4 component.

In some embodiments, the IL-4R activator further comprises a moiety that enhances stability or half-life, including for example an Fc portion or a PEG moiety.

In some embodiments, the plurality of S/D/M factors comprise an IL-4R activator, optionally wherein the IL-4R activator is selected from the group consisting of IL-4, IL-13, an IL-4R agonist antibody, and a small molecule activator of IL-4R. In some embodiments, the IL-4R activator is IL-4. In some embodiments, the IL-4 is a human IL-4 or a human recombinant IL-4. In some embodiments, the IL-4 (e.g., a human IL-4) is present in the medium at a concentration of at least about 15 μg/ml, optionally at least about 30 μg/ml (e.g., at least about 30 μg/ml, 50 μg/ml, 75 μg/ml, 100 μg/ml, 125 μg/ml, or 150 μg/ml). In some embodiments, the IL-4 (e.g., a human IL-4) is present in the medium at a concentration of about 15 μg/ml to about 1.5 ng/ml (e.g., about 30 μg/ml to about 1 ng/ml, e.g., about 100 μg/ml to about 1 ng/ml, e.g., about 100 μg/ml to about 1 ng/ml).

In some embodiments, the IL-4R activator is IL-13 (such as a human IL-13 or a human recombinant IL-13). In some embodiments, the IL-13 is present in the medium at a concentration of at least about 30 μg/ml, optionally at least about 60 μg/ml, further optionally about 60 μg/ml to about 2 ng/ml (e.g., about 100 μg/ml to about 2 ng/ml).

### TNFα Receptor (TNFR) Activator and TNFα

“TNFα receptor (TNFR) activator” described herein refers to a molecule that activates TNFR mediated signaling pathway. TNFR described herein refers to either TNFR1 or TNFR2.

Tumor necrosis factor α (TNF, cachexin, or cachectin; often called tumor necrosis factor alpha or TNF-α) is an adipokine and a cytokine. TNFα is a member of the TNFα superfamily, which consists of various transmembrane proteins with a homologous TNFα domain.

TNFα can bind two receptors, TNFR1 (TNFα receptor type 1; CD120a; p55/60) and TNFR2 (TNFα receptor type 2; CD120b; p75/80). TNFR1 is 55-kDa and TNER2 is 75-kDa.[38] TNFR1 is expressed in most tissues, and can be fully activated by both the membrane-bound and soluble trimeric forms of TNF, whereas TNFR2 is found typically in cells of the immune system, and responds to the membrane-bound form of the TNFα homotrimer.

TNFα was thought to be produced primarily by macrophages.[50] but it is produced also by a broad variety of cell types including lymphoid cells, mast cells, endothelial cells, cardiac myocytes, adipose tissue, fibroblasts, and neurons.[51] [unreliable medical source?] Large amounts of TNFα are released in response to lipopolysaccharide, other bacterial products, and interleukin-1 (IL-1). In the skin, mast cells appear to be the predominant source of pre-formed TNFα, which can be released upon inflammatory stimulus (e.g., LPS).

TNFα referred herein comprises any constructs that have a component of TNFα (e.g., a naturally occurring TNFα, e.g., a recombinant TNFα). These include and are not limited to natural TNFα (e.g., various isoforms of human TNFα), synthetic or recombinant TNFα, and fusion proteins having a TNFα component.

In some embodiments, the TNFR activator further comprises a moiety that enhances stability or half-life, including for example an Fc portion or a PEG moiety.

In some embodiments, the plurality of S/D/M factors comprise a TNFR activator, optionally wherein the TNFR activator is selected from the group consisting of TNFα, a TNFR agonist antibody, and a small molecule activator of TNFR. In some embodiments, the TNFR activator is TNFα. In some embodiments, the TNFα is a human TNFα or a human recombinant TNFα. In some embodiments, the TNFα (e.g., a human TNFα) is present in the medium at a concentration of at least about 0.2 ng/ml, optionally at least about 0.5 ng/ml (e.g., at least about 1 ng/ml, about 2 ng/ml, or about 3 ng/ml). In some embodiments, the TNFα (e.g., a human TNFα) is present in the medium at a concentration of about 0.2 ng/ml to about 30 ng/ml (e.g., about 0.5 ng/ml to about 10 ng/ml, e.g., about 1 ng/ml to about 5 ng/ml, e.g., about 2 ng/ml to about 4 ng/ml).

### IFNγ Receptor (IFNGR) Activator and IFNγ

“IFNγ receptor (INFGR) activator” described herein refers to a molecule that activates INFGR mediated signaling pathway.

Interferon gamma (IFN-γ) is a dimerized soluble cytokine that is the only member of the type II class of interferons. IFN-γ, or type II interferon, is a cytokine that is critical for innate and adaptive immunity against viral, some bacterial and protozoan infections. IFN-γ is an important activator of macrophages and inducer of major histocompatibility complex class II molecule expression. Aberrant IFN-γ expression is associated with a number of autoinflammatory and autoimmune diseases. The importance of IFN-γ in the immune system stems in part from its ability to inhibit viral replication directly, and most importantly from its immunostimulatory and immunomodulatory effects. IFN-γ is produced predominantly by natural killer cells (NK) and natural killer T cells (NKT) as part of the innate immune response, and by CD4 Th1 and CD8 cytotoxic T lymphocyte (CTL) effector T cells once antigen-specific immunity develops as part of the adaptive immune response. IFN-γ is also produced by non-cytotoxic innate lymphoid cells (ILC), a family of immune cells first discovered in the early 2010s.

IFN-γ referred herein comprises any constructs that have a component of IFN-γ (e.g., a naturally occurring IFN-γ, e.g., a recombinant IFN-γ). These include and are not limited to natural IFN-γ (e.g., various isoforms of human IFN-γ), synthetic or recombinant IFN-γ, and fusion proteins having an IFN-γ component.

In some embodiments, the IFNGR activator further comprises a moiety that enhances stability or half-life, including for example an Fc portion or a PEG moiety.

In some embodiments, the plurality of S/D/M factors comprise an IFNGR activator, optionally wherein the IFNGR activator is selected from the group consisting of IFNγ, an IFNGR agonist antibody, and a small molecule activator of IFNGR. In some embodiments, the IFNGR activator is IFNγ. In some embodiments, the IFNγ is a human IFNγ or a human recombinant IFNγ. In some embodiments, the IFNγ (e.g., a human IFNγ) is present in the medium at a concentration of at least about 1 ng/ml, optionally at least about 5 ng/ml (e.g., at least about 10 ng/ml, about 20 ng/ml, or about 50 ng/ml). In some embodiments, the IFNγ (e.g., a human IFNγ) is present in the medium at a concentration of about 1 ng/ml to about 500 ng/ml (e.g., about 5 ng/ml to about 200 ng/ml, e.g., about 10 ng/ml to about 100 ng/ml, e.g., about 40 ng/ml to about 60 ng/ml, e.g., about 50 ng/ml).

In some embodiments, the plurality of S/D/M factors comprise two or more agents selected from the group consisting of an IL-4R activator, a TNFR activator, and an IFNGR activator as described herein.

In some embodiments, the plurality of S/D/M factors comprises IL-10, IL-4, TNFα, and IFNγ.

### GM-CSF Receptor (GM-CSFR) Activator and GM-CSF

“GM-CSF receptor (GM-CSFR) activator” described herein refers to a molecule that activates GM-CSFR mediated signaling pathway.

Granulocyte-macrophage colony-stimulating factor (GM-CSF), also known as colony-stimulating factor 2 (CSF2), is a monomeric glycoprotein secreted by macrophages, T cells, mast cells, natural killer cells, endothelial cells and fibroblasts that functions as a cytokine. GM-CSF stimulates stem cells to produce granulocytes (neutrophils, cosinophils, and basophils) and monocytes. Monocytes exit the circulation and migrate into tissue, whereupon they mature into macrophages and dendritic cells. It is part of the immune/inflammatory cascade, by which activation of a small number of macrophages can rapidly lead to an increase in their numbers, a process crucial for fighting infection. GM-CSF also has some effects on mature cells of the immune system. These include, for example, enhancing neutrophil migration and causing an alteration of the receptors expressed on the cells surface.

GM-CSF referred herein comprises any constructs that have a component of GM-CSF (e.g., a naturally occurring GM-CSF, e.g., a recombinant GM-CSF). These include and are not limited to natural GM-CSF (e.g., various isoforms of human GM-CSF), synthetic or recombinant GM-CSF, and fusion proteins having a GM-CSF component.

In some embodiments, the GM-CSFR activator further comprises a moiety that enhances stability or half-life, including for example an Fc portion or a PEG moiety.

In some embodiments, the plurality of the S/D/M factors further comprise a GM-CSF receptor (GM-CSFR) activator. In some embodiments, the GM-CSFR activator is selected from the group consisting of GM-CSF, a GM-CSFR agonist antibody, and a small molecule activator of GM-CSFR. In some embodiments, the GM-CSFR activator is GM-CSF. In some embodiments, the GM-CSF is a human GM-CSF or a human recombinant GM-CSF. In some embodiments, the GM-CSF (e.g., a human GM-CSF) is present in the medium at a concentration of at least about 30 μg/ml, optionally at least about 50 μg/ml (e.g., at least about 100 μg/ml, about 150 μg/ml, about 200 μg/ml, or about 300 μg/ml). In some embodiments, the GM-CSF (e.g., a human GM-CSF) is present in the medium at a concentration of about 30 μg/ml to about 3 ng/ml (e.g., about 50 μg/ml to about 1 ng/ml, e.g., about 100 μg/ml to about 500 μg/ml, e.g., about 200 μg/ml to about 400 μg/ml, e.g., about 300 μg/ml).

### IL-6 Receptor (IL-6R) Activator and IL-6

“IL-6 receptor (IL-6R) activator” described herein refers to a molecule that activates IL-6 receptor mediated signaling pathway.

Interleukin 6 (IL-6) is an interleukin that acts as both a pro-inflammatory cytokine and an anti-inflammatory myokine. In immune system, IL-6 is secreted by macrophages in response to specific microbial molecules, referred to as pathogen-associated molecular patterns (PAMPs). These PAMPs bind to an important group of detection molecules of the innate immune system, called pattern recognition receptors (PRRs), including Toll-like receptors (TLRs). These are present on the cell surface and intracellular compartments and induce intracellular signaling cascades that give rise to inflammatory cytokine production. IL-6 is an important mediator of fever and of the acute phase response. IL-6 is responsible for stimulating acute phase protein synthesis, as well as the production of neutrophils in the bone marrow. It supports the growth of B cells and is antagonistic to regulatory T cells.

IL-6 referred herein comprises any constructs that have a component of IL-6 (e.g., a naturally occurring IL-6, e.g., a recombinant IL-6). These include and are not limited to natural IL-6 (e.g., various isoforms of human IL-6), synthetic or recombinant IL-6, and fusion proteins having an IL-6 component.

In some embodiments, the IL-6R activator further comprises a moiety that enhances stability or half-life, including for example an Fc portion or a PEG moiety.

In some embodiments, the plurality of the S/D/M factors further comprise an IL-6 receptor (IL-6R) activator, optionally wherein the IL-6R activator is selected from the group consisting of IL-6, an IL-6R agonist antibody, and a small molecule activator of IL-6R. In some embodiments, the IL-6R activator is IL-6. In some embodiments, the IL-6 is a human IL-6 or a human recombinant IL-6. In some embodiments, the IL-6 (e.g., a human IL-6) is present in the medium at a concentration of at least about 1 μg/ml, optionally at least about 5 μg/ml (e.g., at least about 10 μg/ml, about 15 μg/ml, about 20 μg/ml, or about 25 μg/ml). In some embodiments, the IL-6 (e.g., a human IL-6) is present in the medium at a concentration of about 1 μg/ml to about 300 μg/ml (e.g., about 5 μg/ml to about 100 μg/ml, e.g., about 10 μg/ml to about 50 μg/ml, e.g., about 20 μg/ml to about 40 μg/ml, e.g., about 30 μg/ml).

In some embodiments, the plurality of S/D/M factors comprises IL-10, IL-4, TNFα, IFNγ, GM-CSF and IL-6.

In some embodiments, the plurality of S/D/M factors described herein is present in a single composition.

In some embodiments, the plurality of S/D/M factors described herein further comprises one or more cytokines selected from the group consisting of IL-2, IL-17 (e.g., IL-17A), and/or M-CSF.

In some embodiments, at least one of the plurality of S/D/M factors (e.g., IL-10) is provided separately from other S/D/M factors in the plurality of S/D/M factors.

### Refinement Factors

In some embodiments, the methods described above further comprises contacting the population of monocytes with a plurality of refinement factors after the plurality of monocytes are contacted with the plurality of S/D/M factors or the medium derived from the culture of T cells. The refinement factors are selected from the group consisting of type-I interferon (such as IFNα and/or IFNβ), IFNγ, TNFα, a TLR ligand (such as poly IC, CpG, or LPS), CD40L or a CD40-ligating antibody, an anti-PD-L1 antibody, and TPI-1.

In some embodiments, there is provided a method of refining a population of APCs, comprising contacting the population of APCs with a) IFNα, b) IFNγ, and c) TNFα.

In some embodiments, there is provided a method of refining a population of APCs, comprising contacting the population of APCs with a) IFNα, b) IFNγ, c) TNFα, d) poly IC and e) CpG.

In some embodiments, there is provided a method of refining a population of APCs, comprising contacting the population of APCs with a) IFNα, b) IFNγ, c) TNFα, d) poly IC, e) CD40L, and f) anti-PD-L1 antibody.

In some embodiments, there is provided a method of refining a population of APCs, comprising contacting the population of APCs with a) IFNα, b) IFNγ, c) TNFα, d) poly IC, e) CD40L, f) anti-PD-L1 antibody, g) a SHP-1 inhibitor (e.g., TPI-1).

In some embodiments, there is provided a method of refining a population of APCs, comprising contacting the population of APCs with a) IFNα, b) IFNγ, c) R848, d) poly IC, e) a SHP-1 inhibitor (e.g., TPI-1).

In some embodiments, there is provided a method of refining a population of APCs, comprising contacting the population of APCs with a) IFNγ, b) R848, c) poly IC, d) a SHP-1 inhibitor (e.g., TPI-1).

In some embodiments, there is provided a method of refining a population of APCs, comprising contacting the population of APCs with a) IFNα, b) IFNγ, c) poly IC, d) CpG, e) CD40L, f) anti-PD-L1 antibody, g) a SHP-1 inhibitor (e.g., TPI-1), and h) TNFα.

In some embodiments, the plurality of the refinement factors are provided immediately after the plurality of monocytes are contacted with the plurality of S/D/M factors or the medium derived from the culture of T cells. In some embodiments, the plurality of the refinement factors are provided within about one day after the plurality of monocytes are contacted with the plurality of S/D/M factors or the medium derived from the culture of T cells.

In some embodiments, the plurality of the monocytes are cultured for about 1-5 days (e.g., for about one, two, three, four or five days) in the presence of the plurality of the refinement factors.

In some embodiments, the plurality of refinement factors are provided when at least about 50% (e.g., about 50%, 60%, 70%, 80%, or 99%) of the monocytes survive after the plurality of monocytes are contacted with the plurality of S/D/M factors or the medium derived from the culture of T cells.

In some embodiments, the plurality of refinement factors are provided when at least about 10%, 20%, 30%, 40% or 50% of the monocytes exhibit a dendritic cell morphology.

In some embodiments, the plurality of refinement factors are provided when monocytes express a high level of one or more molecules selected from the group consisting of MHC I, MHC II, CD80, CD86, and/or CD40. In some embodiments, the plurality of refinement factors are provided when monocytes express a higher (e.g., at least 20%, 30%, 40%, 50%, 60%, 70%, 80%, 90%, or 100%) level of one or more molecules selected from the group consisting of MHC I, MHC II, CD80, CD86, and/or CD40 than monocytes obtained from the same individual and cultured with GM-CSF and M-CSF (e.g., at a concentration routinely used in the field for such methods).

In some embodiments, the plurality of refinement factors described herein can be used independently from the methods described above for priming APCs (e.g., APCs obtained from a human).

In some embodiments, the refinement factors comprise IFNα, IFNγ, and TNFα.

In some embodiments, the refinement factors comprise IFNα, IFNγ, TNFα, poly IC and CpG.

In some embodiments, the refinement factors comprise IFNα, IFNγ, TNFα, poly IC, CpG, CD40L, and an anti-PD-L1 antibody.

In some embodiments, the refinement factors comprise IFNα, IFNγ, TNFα, poly IC, CpG, CD40L, an anti-PD-L1 antibody and TPI-1.

In some embodiments, the concentration of IFNα in the refinement factors is about 1 ng/ml to about 50 ng/ml (e.g., about 5 ng/ml to about 20 ng/ml, e.g., about 10 ng/ml).

In some embodiments, the concentration of IFNγ in the refinement factors is about 5 ng/ml to about 500 ng/ml (e.g., about 10 ng/ml to about 250 ng/ml, e.g., about 20 ng/ml to about 100 ng/ml, e.g., about 50 ng/ml).

In some embodiments, the concentration of TNFα in the refinement factors is about 1 ng/ml to about 50 ng/ml (e.g., about 5 ng/ml to about 20 ng/ml, e.g., about 10 ng/ml).

In some embodiments, the concentration of poly IC in the refinement factors is about 0.1 μg/ml to about 10 μg/ml (e.g., about 0.2 μg/ml to about 5 μg/ml, e.g., about 0.5 μg/ml to about 2.5 μg/ml, e.g., about 1 μg/ml).

In some embodiments, the concentration CpG of in the refinement factors is about 0.1 μg/ml to about 10 μg/ml (e.g., about 0.2 μg/ml to about 5 μg/ml, e.g., about 0.5 μg/ml to about 2.5 μg/ml, e.g., about 1 μg/ml).

In some embodiments, the concentration of CD40L in the refinement factors is about 1 μg/ml to about 100 μg/ml (e.g., about 2 μg/ml to about 50 μg/ml, e.g., about 5 μg/ml to about 20 μg/ml, e.g., about 10 μg/ml).

In some embodiments, the concentration of the anti-PD-L1 antibody in the refinement factors is about 1 μg/ml to about 200 μg/ml (e.g., about 5 μg/ml to about 100 μg/ml, e.g., about 10 μg/ml to about 50 μg/ml, e.g., about 20 μg/ml).

In some embodiments, the concentration of TPI-1 in the refinement factors is about 0.1 μg/ml to about 10 μg/ml (e.g., about 0.2 μg/ml to about 5 μg/ml, e.g., about 0.5 μg/ml to about 2.5 μg/ml, e.g., about 1 μg/ml).

In some embodiments, the concentration of R848 in the refinement factors is about 0.1 μg/ml to about 10 μg/ml (e.g., about 0.2 μg/ml to about 5 μg/ml, e.g., about 0.5 μg/ml to about 2.5 μg/ml, e.g., about 1 μg/ml).

### Methods for Promoting Survival of the Monocytes

The present application provides various methods for promoting survival of monocytes. In some embodiments, the monocytes are obtained (e.g., freshly isolated) from an individual (e.g., a human). In some embodiments, the individual has a cancer (e.g., any type or kind of cancer described herein). In some embodiments, the individual has a disease or condition associated with immune suppression (e.g., fibrosis, post organ transplantation under immunosuppression medications). In some embodiments, the individual has a virus infection. In some embodiments, the monocytes obtained from the individual express a lower level of IL-10 receptor (“IL-10R”), IL-4 receptor (“IL-4R”), IL-6 receptor (“IL-6R”), M-CSF receptor (“GM-CSFR”), and/or M-CSF receptor (“GM-CSFR”) as compared to those obtained from a reference individual (e.g., a healthy individual).

In some embodiments, the present application provides a method of promoting the survival of a population of monocytes from an individual in an in vitro culture, comprising cultivating the population of monocytes in a medium having an IL-10R activator, optionally wherein the IL-10R activator is selected from the group consisting of: an IL-10 (e.g., a pegylated IL-10, e.g., pegilodecakin or AM0010), an IL-10 family member (e.g., IL-19, IL-20, IL-22, IL-24, IL-26, IL-28), an IL-10R agonist antibody, a small molecule activator of IL-10R, and an activator of the IL-10R downstream STAT3 (e.g., Long noncoding RNA (LncRNA) PVT1, NEAT1, FEZF1-AS1, UICC). In some embodiments, the IL-10 is a human IL-10 or a human recombination IL-10. In some embodiments, the IL-10 is present in the medium at a concentration of at least about 2 ng/ml, optionally at least about 10 ng/ml, further optionally about 10 ng/ml to about 200 ng/ml (e.g., about 20 ng/ml). In some embodiments, the population of monocytes express a lower level (at least about 10%, 15%, 20%, 25%, 30%, 35%, 40%, 45% or 50% lower) of IL-10R prior to contacting with the IL-10R activator as compared to monocytes obtained from a reference individual (e.g., a healthy individual). In some embodiments, the culture further comprise a TNFα receptor (TNFR) activator, and/or an interferon γ (IFNγ) receptor (IFNGR) activator, optionally wherein the TNFR activator is selected from the group consisting of TNFα, a TNFR agonist antibody, and a small molecule activator of TNFR, and optionally wherein the IFNGR activator is selected from the group consisting of IFNγ, an IFNGR agonist antibody, and a small molecule activator of IFNGR, and further optionally the culture comprises TNFα and/or IFNγ. In some embodiments, the IFNγ is present in the medium at a concentration of at least about 5 ng/ml, optionally at least about 10 ng/ml, further optionally about 10 ng/ml to about 200 ng/ml (e.g., about 50-100 ng/ml). In some embodiments, the TNFα is present in the medium at a concentration of at least about 0.5 ng/ml, optionally at least about 1 ng/ml, further optionally about 0.5 ng/ml to about 30 ng/ml (e.g., about 1-10 ng/ml). In some embodiments, the individual has a cancer (e.g., a solid tumor).

In some embodiments, the present application provides a method of promoting the survival of a population of monocytes from an individual in an in vitro culture, comprising cultivating the population of monocytes in a medium having a TNFα receptor (TNFR) activator, and/or an interferon γ (IFNγ) receptor (IFNGR) activator, optionally wherein the TNFR activator is selected from the group consisting of TNFα, a TNFR agonist antibody, and a small molecule activator of TNFR, and optionally wherein the IFNGR activator is selected from the group consisting of IFNγ, an IFNGR agonist antibody, and a small molecule activator of IFNGR, and further optionally the culture comprises TNFα and/or IFNγ. In some embodiments, the IFNγ is present in the medium at a concentration of at least about 5 ng/ml, optionally at least about 10 ng/ml, further optionally about 10 ng/ml to about 200 ng/ml (e.g., about 50-100 ng/ml). In some embodiments, the TNFα is present in the medium at a concentration of at least about 0.5 ng/ml, optionally at least about 1 ng/ml, further optionally about 0.5 ng/ml to about 30 ng/ml (e.g., about 1-10 ng/ml). In some embodiments, the population of monocytes express a lower level (at least about 10%, 15%, 20%, 25%, 30%, 35%, 40%, 45% or 50% lower) of IL-10R prior to contacting with the IL-10R activator as compared to monocytes obtained from a reference individual (e.g., a healthy individual). In some embodiments, the individual has a cancer (e.g., a solid tumor).

In some embodiments, the present application provides a method of promoting the survival of a population of monocytes from an individual in an in vitro culture, comprising cultivating the population of monocytes in a medium having IL-10, TNFα and IFNγ. In some embodiments, the IL-10 is a human IL-10 or a human recombination IL-10. In some embodiments, the IL-10 is present in the medium at a concentration of at least about 2 ng/ml, optionally at least about 10 ng/ml, further optionally about 10 ng/ml to about 200 ng/ml (e.g., about 20 ng/ml). In some embodiments, the IFNγ is present in the medium at a concentration of at least about 5 ng/ml, optionally at least about 10 ng/ml, further optionally about 10 ng/ml to about 200 ng/ml (e.g., about 50-100 ng/ml). In some embodiments, the TNFα is present in the medium at a concentration of at least about 0.5 ng/ml, optionally at least about 1 ng/ml, further optionally about 0.5 ng/ml to about 30 ng/ml (e.g., about 1-10 ng/ml). In some embodiments, the individual has a cancer (e.g., a solid tumor).

In some embodiments, the culture further comprises a GM-CSF receptor (GM-CSFR) activator. In some embodiments, the GM-CSFR activator is selected from the group consisting of GM-CSF, a GM-CSFR agonist antibody, and a small molecule activator of GM-CSFR. In some embodiments, the GM-CSFR activator is GM-CSF. In some embodiments, the GM-CSF is a human GM-CSF or a human recombinant GM-CSF. In some embodiments, the GM-CSF is present in the medium at a concentration of at least about 30 μg/ml, optionally at least about 50 μg/ml, further optionally about 100 μg/ml to about 1 ng/ml (e.g., about 100 μg/ml to about 500 μg/ml, e.g., about 300 μg/ml).

In some embodiments, the culture further comprises an IL-6 receptor (IL-6R) activator. In some embodiments, the IL-6R activator is selected from the group consisting of IL-6, an IL-6R agonist antibody, and a small molecule activator of IL-6R. In some embodiments, the IL-6R activator is IL-6. In some embodiments, the IL-6 is a human IL-6 or a human recombinant IL-6. In some embodiments, the IL-6 is present in the medium at a concentration of at least about 1 μg/ml, optionally at least about 5 μg/ml, further optionally about 5 μg/ml to about 100 μg/ml (e.g., about 10-50 pg/ml, e.g., about 30 μg/ml).

In some embodiments, the present application provides a method of promoting the survival of a population of monocytes from an individual in an in vitro culture, comprising cultivating the population of monocytes in a medium derived from a culture of T cells after being treated with anti-CD3 and anti-CD28 antibodies, wherein the medium comprises an activator of IL-10R. In some embodiments, the T cells are CD4 T cells. In some embodiments, the T cells are CD8 T cells. In some embodiments, the T cells are isolated from PBMC of the same individual or a different individual and have not been previously treated with anti-CD3 and/or anti-CD28 antibodies prior to the treatment. In some embodiments, the T cells are isolated from PBMC of the same individual or a different individual and have been previously treated with anti-CD3 and/or anti-CD28 antibodies prior to the treatment. In some embodiments, the medium is derived from the culture after the T cells are treated with anti-CD3 and anti-CD28 antibodies for about 1-3 days, optionally for about 2 days. In some embodiments, the population of monocytes express a lower level (at least about 10%, 15%, 20%, 25%, 30%, 35%, 40%, 45% or 50% lower) of IL-10R prior to contacting with the IL-10R activator as compared to monocytes obtained from a reference individual (e.g., a healthy individual). In some embodiments, the individual has a cancer (e.g., a solid tumor).

The anti-CD3/CD28 treatment for T cells described herein are techniques well known in the field for activating T cells.

The present application further provides a method of increasing expression of IL-10 receptor (IL-10R) in a population of monocytes from an individual having cancer, comprising contacting the population of monocytes with one or more agents selected from the group consisting of: an IL-10R activator, a TNFR activator, and an IFNGR activator. In some embodiments, the population of monocytes express a lower level (at least about 10%, 15%, 20%, 25%, 30%, 35%, 40%, 45% or 50% lower) of IL-10R prior to contacting with the IL-10R activator as compared to monocytes obtained from a reference individual (e.g., a healthy individual). In some embodiments, the IL-10R activator is selected from the group consisting of: an IL-10 (e.g., a pegylated IL-10, e.g., pegilodecakin or AM0010), an IL-10 family member (e.g., IL-19, IL-20, IL-22, IL-24, IL-26, IL-28), an IL-10R agonist antibody, a small molecule activator of IL-10R, and an activator of the IL-10R downstream STAT3 (e.g., Long noncoding RNA (LncRNA) PVT1, NEAT1, FEZF1-AS1, UICC). In some embodiments, the IL-10R activator is IL-10. In some embodiments, the IL-10 is a human IL-10 or a human recombination IL-10. In some embodiments, the IL-10 is present in the medium at a concentration of at least about 2 ng/ml, optionally at least about 10 ng/ml, further optionally about 10 ng/ml to about 200 ng/ml (e.g., about 20 ng/ml). In some embodiments, the population of monocytes express a lower level (at least about 10%, 15%, 20%, 25%, 30%, 35%, 40%, 45% or 50% lower) of IL-10R prior to contacting with the IL-10R activator as compared to monocytes obtained from a reference individual (e.g., a healthy individual). In some embodiments, the the TNFR activator is selected from the group consisting of TNFα, a TNFR agonist antibody, and a small molecule activator of TNFR. In some embodiments, the IFNGR activator is selected from the group consisting of IFNγ, an IFNGR agonist antibody, and a small molecule activator of IFNGR. In some embodiments, the IFNGR activator comprises TNFα. In some embodiments, the IFNGR activator comprises IFNγ. In some embodiments, the IFNγ is present in the medium at a concentration of at least about 5 ng/ml, optionally at least about 10 ng/ml, further optionally about 10 ng/ml to about 200 ng/ml (e.g., about 50-100 ng/ml). In some embodiments, the TNFα is present in the medium at a concentration of at least about 0.5 ng/ml, optionally at least about 1 ng/ml, further optionally about 0.5 ng/ml to about 30 ng/ml (e.g., about 1-10 ng/ml). In some embodiments, the individual has a cancer (e.g., a solid tumor).

In some embodiments, the monocytes are cultured for at least about 2 days (e.g., about 2-3 days).

### Methods for Promoting the Differentiation of a Population of Monocytes to Antigen Presenting Cells (“APCs”)

The present application provides a method of promoting the differentiation of a population of monocytes from an individual (e.g., a cancer patient or a patient with virus infection) to antigen presenting cells (“APCs”) in an in vitro culture, comprising cultivating the population of monocytes in a medium having one or more molecules selected from the group consisting of an IL-4 receptor (IL-4R) activator (e.g., IL-4), a TNFα receptor (TNFR) activator (e.g., TNFα), and an interferon γ (IFNγ) receptor (IFNGR) activator (e.g., IFNγ), optionally wherein the monocytes have contacted with, or the medium further comprises an IL-10 receptor (IL-10R) activator (e.g., IL-10). In some embodiments, there is provided a method of promoting the differentiation of a population of monocytes from an individual (e.g., a cancer patient) to antigen presenting cells (“APCs”) in an in vitro culture, comprising cultivating the population of monocytes in a medium having an IL-4 receptor (IL-4R) activator (e.g., IL-4), a TNFα receptor (TNFR) activator (e.g., TNFα), and an interferon γ (IFNγ) receptor (IFNGR) activator (e.g., IFNγ). In some embodiments, the IL-4R activator is selected from the group consisting of IL-4, IL-13, an IL-4R agonist antibody, and a small molecule activator of IL-4R. In some embodiments, the IL-4R activator is IL-4. In some embodiments, the IL-4 is a human IL-4 or a human recombinant IL-4. In some embodiments, the IL-4 is present in the medium at a concentration of at least about 15 μg/ml, optionally at least about 30 μg/ml, further optionally about 30 μg/ml to about 1 ng/ml (e.g., about 100 μg/ml to about 1 ng/ml). In some embodiments, the TNFR activator is selected from the group consisting of TNFα, a TNFR agonist antibody, and a small molecule activator of TNFR. In some embodiments, the TNFR activator is TNFα. In some embodiments, the TNFα is a human TNFα or a human recombinant TNFα. In some embodiments, the TNFα is present in the medium at a concentration of at least about 0.5 ng/ml, optionally at least about 1 ng/ml, further optionally about 0.5 ng/ml to about 30 ng/ml (e.g., about 1-10 ng/ml). In some embodiments, the IFNGR activator is selected from the group consisting of IFNγ, an IFNGR agonist antibody, and a small molecule activator of IFNGR. In some embodiments, the IFNGR activator is IFNγ. In some embodiments, the IFNγ is a human IFNγ or a human recombinant IFNγ. In some embodiments, the IFNγ is present in the medium at a concentration of at least about 5 ng/ml, optionally at least about 10 ng/ml, further optionally about 10 ng/ml to about 200 ng/ml (e.g., about 50-100 ng/ml). In some embodiments, the culture further comprises the IL-6 receptor (IL-6R) activator. In some embodiments, the IL-6R activator is selected from the group consisting of IL-6, an IL-6R agonist antibody, and a small molecule activator of IL-6R. In some embodiments, the IL-6R activator is IL-6. In some embodiments, the IL-6 is present in the medium at a concentration of at least about 1 μg/ml, optionally at least about 5 μg/ml, further optionally about 5 μg/ml to about 100 μg/ml (e.g., about 10-50 pg/ml, e.g., about 30 μg/ml). In some embodiments, the monocytes obtained from the individual express a lower (e.g., at least about 10%, 15%, 20%, 25%, 30%, 35%, 40%, 45% or 50% lower) level of IL-4 receptor (“IL-4R”) as compared to those obtained from a reference individual (e.g., a healthy individual).

In some embodiments, there is provided a method of promoting the differentiation of a population of monocytes from an individual (e.g., a cancer patient) to antigen presenting cells (“APCs”) in an in vitro culture, comprising cultivating the population of monocytes in a medium having IL-4, TNFα, and IFNγ. In some embodiments, the IL-4 is a human IL-4 or a human recombinant IL-4. In some embodiments, the IL-4 is present in the medium at a concentration of at least about 15 μg/ml, optionally at least about 30 μg/ml, further optionally about 30 μg/ml to about 1 ng/ml (e.g., about 100 μg/ml to about 1 ng/ml). In some embodiments, the TNFα is a human TNFα or a human recombinant TNFα. In some embodiments, the TNFα is present in the medium at a concentration of at least about 0.5 ng/ml, optionally at least about 1 ng/ml, further optionally about 0.5 ng/ml to about 30 ng/ml (e.g., about 1-10 ng/ml). In some embodiments, the IFNγ is a human IFNγ or a human recombinant IFNγ. In some embodiments, the IFNγ is present in the medium at a concentration of at least about 5 ng/ml, optionally at least about 10 ng/ml, further optionally about 10 ng/ml to about 200 ng/ml (e.g., about 50-100 ng/ml). In some embodiments, the monocytes obtained from the individual express a lower (e.g., at least about 10%, 15%, 20%, 25%, 30%, 35%, 40%, 45% or 50% lower) level of IL-4 receptor (“IL-4R”) as compared to those obtained from a reference individual (e.g., a healthy individual).

In some embodiments, there is provided a method of promoting the differentiation of a population of monocytes from an individual (e.g., a cancer patient) to antigen presenting cells (“APCs”) in an in vitro culture, comprising cultivating the population of monocytes in a medium having IL-4, IL-6, TNFα, and IFNγ, optionally wherein the monocytes have a lower (e.g., at least about 10%, 15%, 20%, 25%, 30%, 35%, 40%, 45% or 50% lower) level of IL-4 receptor (IL-4R) expression as compared to monocytes obtained from a reference individual (e.g., a healthy individual). In some embodiments, the IL-4 is a human IL-4 or a human recombinant IL-4. In some embodiments, the IL-4 is present in the medium at a concentration of at least about 15 μg/ml, optionally at least about 30 μg/ml, further optionally about 30 μg/ml to about 1 ng/ml (e.g., about 100 μg/ml to about 1 ng/ml). In some embodiments, the TNFα is a human TNFα or a human recombinant TNFα. In some embodiments, the TNFα is present in the medium at a concentration of at least about 0.5 ng/ml, optionally at least about 1 ng/ml, further optionally about 0.5 ng/ml to about 30 ng/ml (e.g., about 1-10 ng/ml). In some embodiments, the IFNγ is a human IFNγ or a human recombinant IFNγ. In some embodiments, the IFNγ is present in the medium at a concentration of at least about 5 ng/ml, optionally at least about 10 ng/ml, further optionally about 10 ng/ml to about 200 ng/ml (e.g., about 50-100 ng/ml). In some embodiments, the IL-6 is a human IL-6 or a human recombinant IL-6. In some embodiments, the IL-6 is present in the medium at a concentration of at least about 1 μg/ml, optionally at least about 5 μg/ml, further optionally about 5 μg/ml to about 100 μg/ml (e.g., about 10-50 pg/ml, e.g., about 30 μg/ml).

In some embodiments, the culture further comprises a GM-CSF receptor (GM-CSFR) activator. In some embodiments, the GM-CSFR activator is selected from the group consisting of GM-CSF, a GM-CSFR agonist antibody, and a small molecule activator of GM-CSFR. In some embodiments, the GM-CSFR activator is GM-CSF. In some embodiments, the GM-CSF is a human GM-CSF or a human recombinant GM-CSF. In some embodiments, the GM-CSF is present in the medium at a concentration of at least about 30 μg/ml, optionally at least about 50 μg/ml, further optionally about 100 μg/ml to about 1 ng/ml (e.g., about 100 μg/ml to about 500 μg/ml, e.g., about 300 μg/ml).

In some embodiments, the culture further comprises an IL-10 receptor (IL-10R) activator. In some embodiments, the IL-10R activator is selected from the group consisting of: an IL-10 (e.g., a pegylated IL-10, e.g., pegilodecakin or AM0010), an IL-10 family member (e.g., IL-19, IL-20, IL-22, IL-24, IL-26, IL-28), an IL-10R agonist antibody, a small molecule activator of IL-10R, and an activator of the IL-10R downstream STAT3 (e.g. Long noncoding RNA (LncRNA) PVT1, NEAT1, FEZF1-AS1, UICC). In some embodiments, the IL-10R activator is IL-10. In some embodiments, the IL-10 is a human IL-10 or a human recombinant IL-10. In some embodiments, the IL-10 is a human IL-10 or a human recombination IL-10. In some embodiments, the IL-10 is present in the medium at a concentration of at least about 2 ng/ml, optionally at least about 10 ng/ml, further optionally about 10 ng/ml to about 200 ng/ml (e.g., about 20 ng/ml).

In some embodiments, the monocytes are cultured for at least about 2 days (e.g., about 2-3 days).

### Methods of Obtaining Tumor-Specific or Virus-Specific Antigen Presenting Cells

In some embodiments, there is provided a method of obtaining tumor-specific antigen presenting cells, comprising: a) contacting a population of monocytes obtained from an individual with a plurality of survival, differentiation and/or maturation factors (“S/D/M factors”) separately or simultaneously, wherein the plurality of S/D/M factors comprise: 1) an IL-10 receptor (IL-10R) activator and 2) one or more agents selected from the group consisting of: an IL-4 receptor (IL-4R) activator, a TNFα receptor (TNFR) activator, and an interferon γ (IFNγ) receptor (IFNGR) activator, thereby obtaining a population of APCs; and b) contacting the APCs with a composition comprising a tumor antigen (e.g., a plurality of synthetic tumor antigen peptides), thereby obtaining the tumor-specific APCs.

In some embodiments, the composition comprising a tumor antigen comprises tumor cells (e.g., a tumor biopsy or cells from the patient, e.g., a fresh or a freeze-thaw tumor biopsy sample, e.g., cultured tumor cells from tumor biopsy).

In some embodiments, the composition comprising a tumor antigen comprises neoantigen peptides (e.g., personalized neoantigen long peptides (LP)). In some embodiments, neoantigen peptides are AI-identified and synthesized. See e.g., Mor S K et al. Oncoimmunology. 2022 Jan. 10; 11 (1): 2023255.

In some embodiments, the composition comprising a tumor antigen comprises shared tumor-associated antigens. In some embodiments, the shared tumor-associated antigens comprise self-antigens (e.g., abnormally expressed self-antigens). In some embodiments, the self-antigens are selected from the group consisting of melanoma antigen-1 (MAGE-1), prostate-associated PAP, PSA and PSMA, breast cancer-associated BCAR3, and multi-cancer associated MUC1. In some embodiments, the shared tumor-associated antigens comprise non-self antigens of viral origins (e.g., antigens from LMP1/2 associated with nasopharyngeal carcinoma and lymphoma, e.g., antigens from E6 and E7 proteins of high-risk human papillomavirus (HPV), e.g., antigens from retrovirus Tax protein found in adult T cell leukemia). In some embodiments, the shared tumor-associated antigens comprise mutations-caused neoantigens shared in different types of cancer (e.g., neoantigens associated with p53 mutations or KRAS mutations).

In some embodiments, the tumor antigen peptides (e.g., synthetic tumor antigen peptides) are obtained by: a) identifying a tumor-specific mutation in a tumor tissue sample of a patient having a virus-associated cancer, wherein the tumor-specific mutation is not present in the virus, and b) synthesizing a peptide comprising the tumor-specific mutation. In some embodiments, the tumor-specific mutation is identified by sequencing the tumor tissue sample and a virus sample and comparing the sequences from the two samples.

In some embodiments, the tumor antigen peptides (e.g., synthetic tumor antigen peptides) are obtained by: a) identifying a tumor-specific mutation in a tumor tissue sample of patient having cancer that is not present in a normal tissue sample of the cancer patient, and b) synthesizing a peptide comprising the tumor-specific mutation. In some embodiments, the tumor-specific mutation is identified by sequencing the tumor tissue sample and the normal tissue sample and comparing the sequences from the two samples.

In some embodiments, there is provided a method of obtaining tumor-specific antigen presenting cells, comprising: a) contacting a population of monocytes obtained from an individual with a plurality of survival, differentiation and/or maturation factors (“S/D/M factors”) separately or simultaneously, wherein the plurality of S/D/M factors comprise: 1) an IL-10 receptor (IL-10R) activator and 2) one or more agents selected from the group consisting of: an IL-4 receptor (IL-4R) activator, a TNFα receptor (TNFR) activator, and an interferon γ (IFNγ) receptor (IFNGR) activator, thereby obtaining a population of APCs; and b) introducing into the population of APCs a polynucleotide encoding a tumor antigen (such as any tumor antigen described herein). In some embodiments, the polynucleotide is a DNA. In some embodiments, the polynucleotide is an mRNA.

In some embodiments, there is provided a method of obtaining virus-specific antigen presenting cells, comprising: a) contacting a population of monocytes obtained from an individual with a plurality of survival, differentiation and/or maturation factors (“S/D/M factors”) separately or simultaneously, wherein the plurality of S/D/M factors comprise: 1) an IL-10 receptor (IL-10R) activator and 2) one or more agents selected from the group consisting of: an IL-4 receptor (IL-4R) activator, a TNFα receptor (TNFR) activator, and an interferon γ (IFNγ) receptor (IFNGR) activator, thereby obtaining a population of APCs; and b) contacting the APCs with a composition comprising a virus antigen, thereby obtaining the virus-specific APCs. In some embodiments, the composition comprising a virus antigen comprises a virus sample.

In some embodiments, there is provided a method of obtaining virus-specific antigen presenting cells, comprising: a) contacting a population of monocytes obtained from an individual with a plurality of survival, differentiation and/or maturation factors (“S/D/M factors”) separately or simultaneously, wherein the plurality of S/D/M factors comprise: 1) an IL-10 receptor (IL-10R) activator and 2) one or more agents selected from the group consisting of: an IL-4 receptor (IL-4R) activator, a TNFα receptor (TNFR) activator, and an interferon γ (IFNγ) receptor (IFNGR) activator, thereby obtaining a population of APCs; and b) introducing into the population of APCs a polynucleotide encoding a virus antigen. In some embodiments, the polynucleotide is a DNA. In some embodiments, the polynucleotide is an mRNA.

In some embodiments, there is provided a population of tumor-specific APCs or virus-specific APCs produced by any of the methods described herein.

### Monocytes

The methods described herein converts a plurality of monocytes into APCs. In some embodiments, the plurality of monocytes is obtained from the peripheral blood of the individual.

In some embodiments, the monocytes express CD14 at the time when they are obtained from the peripheral blood. Methods of obtaining monocytes from peripheral blood is well known in the art. For example, PBMC can be planted on cell culture dish to allow monocyte adhesion, which is a common way to separate them from lymphocytes that are not adhesion. Monocytes can also be separated by positive selection with anti-CD14 Ab or negative selection using all Abs against other cells.

The monocytes obtained from the individual in some embodiments express a lower level of IL-10 receptor (“IL-10R”) prior to contacting with one or more cytokines described above as compared to those obtained from a reference individual (e.g., a healthy individual). In some embodiments, the level of IL-10R on the monocytes from the individual is at least about 10%, 15%, 20%, 25%, 30%, 35%, 40%, 45% or 50% lower than the level of IL-10R on monocytes from the reference individual (e.g., a healthy individual).

In some embodiments, the monocytes obtained from the individual express a lower level of IL-4 receptor (“IL-4R”) prior to contacting with one or more cytokines described above as compared to those obtained from a healthy individual. In some embodiments, the level of IL-4R on the monocytes from the individual is at least about 10%, 15%, 20%, 25%, 30%, 35%, 40%, 45% or 50% lower than the level of IL-4R on monocytes from the reference individual (e.g., a healthy individual).

In some embodiments, the monocytes obtained from the individual express a lower level of IL-6 receptor (“IL-6R”) prior to contacting with one or more cytokines described above as compared to those obtained from a healthy individual. In some embodiments, the level of IL-6R on the monocytes from the individual is at least about 10%, 15%, 20%, 25%, 30%, 35%, 40%, 45% or 50% lower than the level of IL-6R on monocytes from the reference individual (e.g., a healthy individual).

In some embodiments, the monocytes obtained from the individual express a lower level of M-CSF receptor (“M-CSFR”) prior to contacting with one or more cytokines described above as compared to those obtained from a healthy individual. In some embodiments, the level of M-CSFR on the monocytes from the individual is at least about 10%, 15%, 20%, 25%, 30%, 35%, 40%, 45%, 50%, 55%, 60%, or 65% lower than the level of M-CSFR on monocytes from the reference individual (e.g., a healthy individual).

In some embodiments, the monocytes obtained from the individual express a lower level of GM-CSF receptor (“GM-CSFR”) prior to contacting with one or more cytokines described above as compared to those obtained from a healthy individual. In some embodiments, the level of GM-CSFR on the monocytes from the individual is at least about 10%, 15%, 20%, 25%, 30%, 35%, 40%, 45%, 50%, 55%, 60%, or 65% lower than the level of GM-CSFR on monocytes from the reference individual (e.g., a healthy individual).

In some embodiments, the methods described herein further comprise assessing IL-10R expression level of the monocytes (e.g., prior to contacting the monocytes with one or more cytokines such as IL-10, IFNγ and/or TNFα).

### Cancer

Cancer described in this section (e.g., in the context of monocytes derived from a cancer patient) can be any type or kind. All the cancer types discussed in Section V are similarly applicable here.

In some embodiments, the cancer is a solid tumor. In some embodiments, the cancer is a hematologic cancer.

In some embodiments, the cancer is an advanced cancer. In some embodiments, the cancer is a late stage cancer. In some embodiments, the cancer is in stage II, III or IV. In some embodiments, the cancer is an inoperable tumor and/or is malignant.

Examples of cancers described herein include, but are not limited to, adrenocortical carcinoma, agnogenic myeloid metaplasia, AIDS-related cancers (e.g., AIDS-related lymphoma), anal cancer, appendix cancer, astrocytoma (e.g., cerebellar and cerebral), basal cell carcinoma, bile duct cancer (e.g., extrahepatic), bladder cancer, bone cancer, (osteosarcoma and malignant fibrous histiocytoma), brain tumor (e.g., glioma, brain stem glioma, cerebellar or cerebral astrocytoma (e.g., pilocytic astrocytoma, diffuse astrocytoma, anaplastic (malignant) astrocytoma), malignant glioma, ependymoma, oligodenglioma, meningioma, craniopharyngioma, hacmangioblastomas, medulloblastoma, supratentorial primitive neuroectodermal tumors, visual pathway and hypothalamic glioma, and glioblastoma), breast cancer, bronchial adenomas/carcinoids, carcinoid tumor (e.g., gastrointestinal carcinoid tumor), carcinoma of unknown primary, central nervous system lymphoma, cervical cancer, colon cancer, colorectal cancer, chronic myeloproliferative disorders, endometrial cancer (e.g., uterine cancer), ependymoma, esophageal cancer, Ewing's family of tumors, eye cancer (e.g., intraocular melanoma and retinoblastoma), gallbladder cancer, gastric (stomach) cancer, gastrointestinal carcinoid tumor, gastrointestinal stromal tumor (GIST), germ cell tumor, (e.g., extracranial, extragonadal, ovarian), gestational trophoblastic tumor, head and neck cancer, hepatocellular (liver) cancer (e.g., hepatic carcinoma and heptoma), hypopharyngeal cancer, islet cell carcinoma (endocrine pancreas), laryngeal cancer, laryngeal cancer, leukemia, lip and oral cavity cancer, oral cancer, liver cancer, lung cancer (e.g., small cell lung cancer, non-small cell lung cancer, adenocarcinoma of the lung, and squamous carcinoma of the lung), lymphoid neoplasm (e.g., lymphoma), medulloblastoma, melanoma, mesothelioma, metastatic squamous neck cancer, mouth cancer, multiple endocrine neoplasia syndrome, myelodysplastic syndromes, myelodysplastic/myeloproliferative diseases, nasal cavity and paranasal sinus cancer, nasopharyngeal cancer, neuroblastoma, neuroendocrine cancer, oropharyngeal cancer, ovarian cancer (e.g., ovarian epithelial cancer, ovarian germ cell tumor, ovarian low malignant potential tumor), pancreatic cancer, parathyroid cancer, penile cancer, cancer of the peritoneal, pharyngeal cancer, pheochromocytoma, pincoblastoma and supratentorial primitive neuroectodermal tumors, pituitary tumor, pleuropulmonary blastoma, lymphoma, primary central nervous system lymphoma (microglioma), pulmonary lymphangiomyomatosis, rectal cancer, renal cancer, renal pelvis and ureter cancer (transitional cell cancer), rhabdomyosarcoma, salivary gland cancer, skin cancer (e.g., non-melanoma (e.g., squamous cell carcinoma), melanoma, and Merkel cell carcinoma), small intestine cancer, squamous cell cancer, testicular cancer, throat cancer, thymoma and thymic carcinoma, thyroid cancer, tuberous sclerosis, urethral cancer, vaginal cancer, vulvar cancer, Wilms' tumor, and post-transplant lymphoproliferative disorder (PTLD), abnormal vascular proliferation associated with phakomatoses, edema (such as that associated with brain tumors), and Meigs' syndrome.

In some embodiments, the cancer is a virus-infection-related cancer. In some embodiments, the cancer is a human papillomavirus (HPV)-related cancer (e.g., HPV-related cervical cancer, e.g., HPV-related head and neck cancer, e.g., HPV related squamous cell carcinoma). In some embodiments, the cancer is human herpes virus 8 (HHV8) related cancer (e.g., Kaposi sarcoma). In some embodiments, the cancer is human T-lymphotrophic virus (HTLV-1)-related cancer (e.g., adult T cell leukemia or lymphoma). In some embodiments, the cancer is Epstein-Barr virus (EBV) related cancer (e.g., Burkitt lymphoma, Hodgkin's and non-Hodgkin's lymphoma, stomach cancer). In some embodiments, the cancer is hepatitis B virus (HBV) related cancer (e.g., liver cancer). In some embodiments, the cancer is hepatitis C virus) related cancer (e.g., liver cancer, non-Hodgkin's lymphoma).

In some embodiments, the cancer is refractory to one or more of irradiation therapy, chemotherapy, or immunotherapy (e.g., checkpoint blockade).

In some embodiments, the cancer is a liver cancer, a kidney cancer, an endometrial cancer, a thymic epithelial neoplasma, lung cancer, spindle cell sarcoma, chondrosarcoma, uterine smooth muscle, pancreatic cancer.

### Individuals

In some embodiments, the individual is healthy or do not exhibit symptoms of a disease or condition (e.g., cancer). In some embodiments, the individual has a cancer or tumor. In some embodiments, the individual has a solid tumor. In some embodiments, the cancer is a hematologic cancer.

In some embodiments, the individual has an advanced cancer. In some embodiments, the individual has a late stage cancer. In some embodiments, the individual has a cancer that is in stage II, III or IV. In some embodiments, the individual has an inoperable tumor and/or metastases. In some embodiments, the cancer is malignant.

In some embodiments, the individual has an infection (e.g., an infection of virus, an infection of a bacteria).

In some embodiments, the individual is a female. In some embodiments, the individual is a male.

In some embodiments, the individual is a human. In some embodiments, the individual is a human who is at least about 50, 55, 60, 65, 70 or 75 years old.

### III. Antigen Presenting Cells (APCs), Compositions, and Cultures

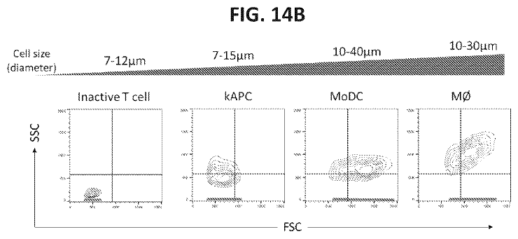

The present application provides APCs such as those prepared according to any of the methods described above with unique properties that distinguish them from naturally occurring APCs or APCs generated in vitro by currently known methods. Comprehensive studies discussed in the Examples in detail show that the exemplified APCs of the present application are distinct from e.g., dendritic cells (e.g., cDC1, cDC2, pDC) or macrophages (e.g., M1 macrophages, M2 macrophages) in shape/size, how they adhere to matrix substratum, various cell surface molecules, antigen presentation capacity, and/or gene transcription profile. See e.g., FIGS. 14-19C. The results as a whole suggest that the exemplified APCs of the present application are a unique population of cells that are generally smaller (about 7-15 μm after trypsinization) than macrophages/dendritic cells (about 10-20 μm) with distinct shapes while having superior antigen presenting capacity and being highly sensitive for various kinds of antigens evidenced by their high expressions of e.g., CD40, TLR2, and STING. Exemplified APCs uniquely express high levels of LOX1, and uPAR and have vastly different gene transcription profiles as compared to mature DCs or macrophages. These data demonstrate that the APCs of the present application represent a novel and robust APC population that is fundamentally distinct from currently known APCs.

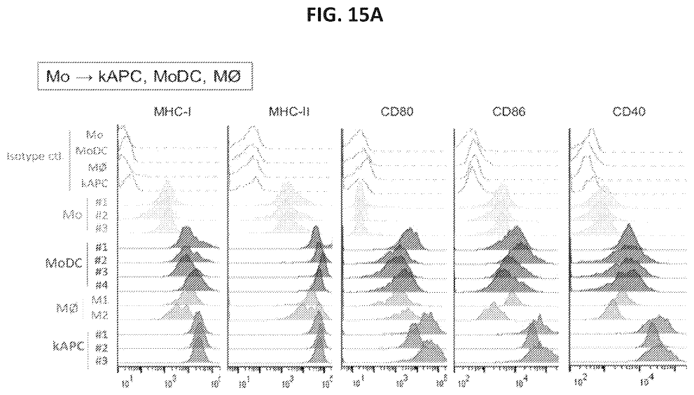

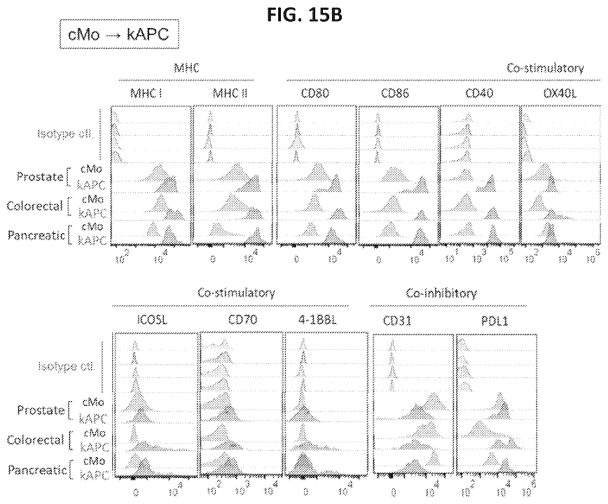

The exemplified APCs described herein have distinct surface markers from known dendritic cell subsets such as myeloid cDC1, myeloid cDC2, plasmacytoid DC (pDC) or Mo-DC, and known human macrophages (such as M1 macrophages and M2 macrophages) or monocytes. Sec e.g., FIGS. 18A-18B, 19A-19C and 15A-15B. For example, while cDC1 express high levels of CD141, CLEC9A, XCR1, CD103, CD103 and DEC205, exemplified APCs of the present application do not express or express a level that is much lower all of the cDC1 markers described above. Similarly, cDC2 express a high level of SIPRα and CD1c, while exemplified APCs of the present application express a level that is much lower of both of them. pDC express a high level of CD303, while exemplified APCs of the present application express a level that is much lower of CD303. MoDC express a high level of DEC205, SIRPα, CD1c, CD11c, CD303, CD209, while exemplified APCs of the present application either do not express or express a much lower level of any of those surface markers. M1 macrophages express a high level of CD26, SIRPα, CD11c, CCR2, CCR7, CD14, CD303, PD-L1, CLEC5, CCR1, while exemplified APCs of the present application have similar expression of CD14 and PD-L1 but have distinct expressions of all other surface markers.

On the other hand, exemplified APCs of the present application consistently express several surface molecules that are not expressed or expressed at a relatively low level in dendritic cells or macrophages described above. These molecules include receptor for oxidized LDL (LOX1), receptor for urokinase plasminogen activator (uPAR), receptor for IL-3 (IL-3R) and receptor for complement component 3a (C3AR), TLR2 and/or STING. None of the dendritic cells or macrophages have a similar expression pattern of these molecules.

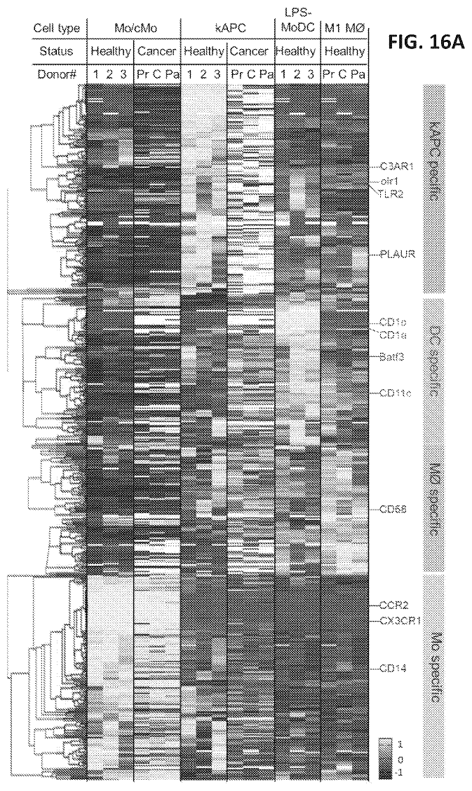

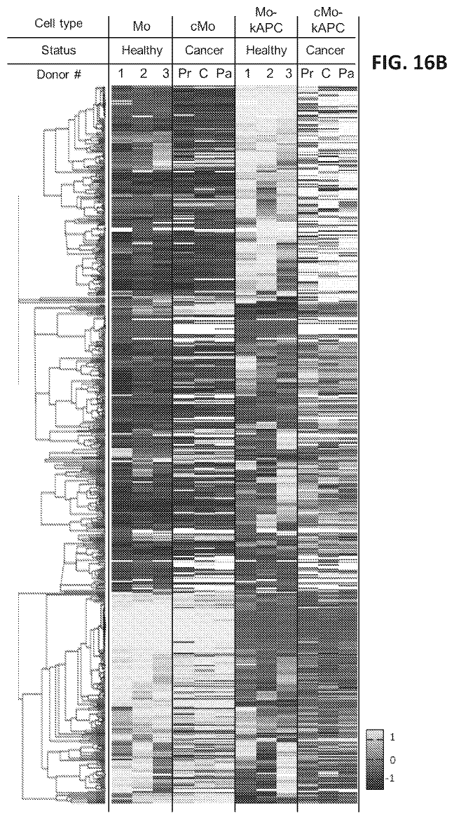

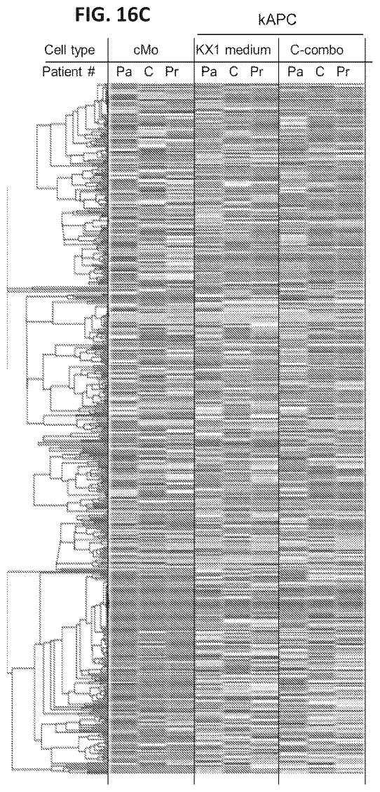

Consistently, exemplified APCs of the present application have distinct morphology as shown in e.g., FIG. 14. These APCs also show distinct gene transcription profile as shown in FIG. 16A-16C. As shown, these cells are smaller than dendritic cells or macrophages and have distinct cell shape. While these exemplified APCs have a unique pattern of gene transcription profile from dendritic cells, macrophages or monocytes, the exemplified APCs a) derived monocytes from cancer patients, b) derived from monocytes from healthy individuals, c) cultured with KX1 d) cultured with c-combo exhibit highly similar gene transcription profiles. These results again evidenced that the APCs of the present application are a unique and non-transient APC population.

In some embodiments, there is provided a population of APCs, wherein the APCs a) are MHC-I+/high and MHC-II+/high, b) express or express a high level of at least one or more (e.g., two, three, four) costimulatory molecules comprising CD40, CD80 and CD86, and/or OX40L+/high, and/or PD-L1+/high, c) TLR2+ and/or STING+, d) do not express or express a lower level of at least one or more (e.g., two, three, four, five, six) cDC1 surface molecules comprising CD141, Clec9a, CD26, XCR1, CD103, DEC205 as compared to cDC1, e) express a lower level of at least one cDC2 surface molecules comprising SIRPα and CD1c as compared to cDC2, f) express a lower level of at least one (e.g., two, three, four, or five) dendritic cell or macrophage surface molecules comprising CD11c, CCR2, CCR7, CD14, and CD303 as compared to MoDC (such as those derived from monocytes after treatment of LPS or TNFα), g) express a lower level of pDC surface molecule CD303, and/or h) express a high level of uPAR and/or LOX1 (e.g., express a higher level of LOX1 and/or uPAR as compared to monocytes, LPS-MoDC, or M1/M2 macrophages). In some embodiments, the APCs are OX40L+/high, ICOSL+, CD70+, and/or 4-1BBL+ or have increased expression of OX40L, ICOSL, CD70, and/or 4-1BBL as compared to monocytes (e.g., the monocytes that they are derived from). In some embodiments, the APCs are CD31+/low or have decreased CD31 as compared to monocytes (e.g., the monocytes that they are derived from). In some embodiments, the APCs are PD-L1+/high or have increased PD-L1 as compared to monocytes (e.g., the monocytes that they are derived from). In some embodiments, the APCs are SIRPα−/low, LilRB−/low, and/or Siglec−/low or have reduced expression of SIRPα, LilRB, and Siglec as compared to monocytes (e.g., the monocytes that they are derived from). In some embodiments, the APCs are CD32+/high, Trem2high, IL-3Rhigh, and/or c-Met+/high. In some embodiments, the APCs are TLR3+/high and/or TLR8+/high (e.g., have a higher TLR3 and/or TLR8 expression than M1 macrophages). In some embodiments, the APCs express a lower level of CD14 as compared to monocytes. In some embodiments, the APCs have a size of less than about 15 μm (diameter) (e.g., about 7-15 μm). In some embodiments, the APCs have multiple shapes (multi-shaped) when they adhere to the matrix, and can elongate/stretch to adopt a spindle shape. After trypsinization, these cells round up and have a uniformed size that is less than about 15 μm (e.g., about 7-15 μm).

“+/high” described herein refers to a positive expression of a certain surface molecule, or a high expression of a certain surface molecule. In some embodiments, a high expression refers to the scenario that the cell (e.g., APCs) expresses a higher level of the surface molecule than a reference cell population. The reference cell population in some embodiments are monocytes, macrophages (e.g., M1 macrophages, or M2 macrophages), dendritic cells (e.g., Mo-DC, cDC1, cDC2, pDC) that are known to express this surface molecule. In some embodiments, a higher level refers to an expression level that is at least about 10%, 20%, 30%, 40%, 50%, 60%, 70%, 80%, 90%, 100%, 2-fold, 5-fold, 10-fold or 100-fold higher level of expression. In some embodiments, an increased expression of a certain molecule refers to an expression level that is at least about 10%, 20%, 30%, 40%, 50%, 60%, 70%, 80%, 90%, 100%, 2-fold, 5-fold, 10-fold or 100-fold higher level of expression. In some embodiments, an decreased expression of a certain molecule refers to an expression level that is at least about 10%, 20%, 30%, 40%, 50%, 60%, 70%, 80% less.

In some embodiments, there is provided a population of APCs, wherein the APCs are LOX1+/high, uPAR+/high, CD40+/high, and/or TLR2+/high. In some embodiments, the APCs have a CD40 surface expression level that is at least 5-fold or 10-fold higher than dendritic cells (e.g., dendritic cells derived from monocyte after GM-CSF/IL-4 treatment followed by LPS treatment as described herein) or M1/M2 macrophages (e.g., M1/M2 macrophages derived from monocytes after M-CSF treatment followed by LPS and IFNγ treatment for M1 phenotype, or LPS, IL4 and IL-10 treatment for M2 phenotype). In some embodiments, the APCs have comparable or higher surface expression of MHC-I, MHC-II, CD80, CD86, and CD40 as compared to dendritic cells (e.g., dendritic cells derived from monocyte after GM-CSF/IL-4 treatment followed by LPS treatment as described herein) or M1/M2 macrophages (e.g., M1/M2 macrophages derived from monocytes after M-CSF treatment followed by LPS and IFNγ treatment for M1 phenotype, or LPS, IL4 and IL-10 treatment for M2 phenotype). In some embodiments, the APCs are IL-3R+/high, TREM2+/high, C3AR+/high, and PD-L1+/high. In some embodiments, the APCs have a size of less than about 15 μm (diameter) (e.g., about 7-15 μm). In some embodiments, the APCs have multiple shapes (multi-shaped) when they adhere to the matrix, and can elongate/stretch to adopt a spindle shape. After trypsinization, these cells round up and have a uniformed size that is less than about 15 μm (e.g., about 7-15 μm).

In some embodiments, there are provided a population of APCs (such as APCs derived from monocytes obtained from an individual (e.g., a healthy individual, a cancer patient or a virus infected patient)), wherein the APCs a) are MHC-I+/high and MHC-II+/high (e.g., express a higher level of MHC-I and MHC-II as compared to monocytes, M1 macrophages, M2 macrophages, and MoDCs), and b) have a size of less than about 15 μm (e.g., about 7-15 μm). In some embodiments, the APCs have multiple shapes (multi-shaped) when they adhere to the matrix, and can elongate/stretch to adopt a spindle shape. After trypsinization, these cells round up and have a uniformed size that is less than about 15 μm (e.g., about 7-15 μm). They are smaller than both dendritic cells and macrophages. See e.g., FIG. 14. In some embodiments, they are TLR2+/high and STING+/high (e.g., express a higher level of TLR2 and STING than M1 macrophages), and/or express a higher level of CD40 as compared to monocytes, M1 macrophages, M2 macrophages, and MoDCs. In some embodiments, the APCs a) do not express or express a lower level of CD141, XCR1, and CD103 as compared to cDC1, b) do not express or express a lower level of CD1c and SIRPα as compared to cDC2, c) express a lower level of CD303 as compared to pDC or M1 macrophages, d) express a lower level of CCR7, CCR2 and CD11c as compared to M1 macrophages. In some embodiments, the APCs have a size of less than about 15 μm (e.g., about 7-15 μm). In some embodiments, the APCs have a spindle or elongated shape, or multi-shaped. In some embodiments, the APCs adhere to the matrix substratum but can be easily dislodged by repeated pipetting or brief trypsinization (<2 min at 37° C.). In some embodiments, the APCs a) are CD80+/high and CD86+/high (e.g., express a higher level of CD80 and CD86 as compared to monocytes, M1 macrophages, M2 macrophages, and MoDCs). In some embodiments, the expression level of CD40 on the APCs are at least 5-fold, 10-fold, 20-fold, 50-fold, or 100-fold higher than that on monocytes, M1 macrophages, M2 macrophages, and MoDCs.

In some embodiments, there are provided a population of APCs (such as APCs derived from monocytes obtained from an individual (e.g., a healthy individual, a cancer patient or a virus infected patient)), wherein the APCs 1) are MHC-I+/high and MHC-II+/high (e.g., express a higher level of MHC-I and MHC-II as compared to monocytes, M1 macrophages, M2 macrophages, and MoDCs), and 2) a) do not express or express a lower level of CD141, XCR1, and CD103 as compared to cDC1, b) do not express or express a lower level of CD1c and SIRPα as compared to cDC2, c) express a lower level of CD303 as compared to pDC or M1 macrophages, and/or d) express a lower level of CCR7, CCR2 and CD11c as compared to M1 macrophages. In some embodiments, the APCs have a size of less than about 15 μm (e.g., about 7-15 μm). In some embodiments, the APCs a) are CD80+/high and CD86+/high (e.g., express a higher level of CD80 and CD86 as compared to monocytes, M1 macrophages, M2 macrophages, and MoDCs). In some embodiments, the APCs express a comparable or higher level of CD80, CD86, CD40, OX40L, ICOSL, and/or CD70 as compared to mature dendritic cells (e.g., derived from monocytes after culture with LPS or TNFα). IN some embodiments, the APC express a higher level of LOX1, uPAR, CD40, TLR2, IL-3R, TREM2, C3AR, IL-3R and/or PD-L1 as compared to mature dendritic cells (e.g., derived from monocytes after culture with LPS or TNFα), monocytes, M1 or M2 macrophages.

In some embodiments, there are provided a population of APCs (such as APCs derived from monocytes obtained from an individual (e.g., a healthy individual, a cancer patient or a virus infected patient)), wherein the APCs a) are MHC-I+/high and MHC-II+/high (e.g., express a higher level of MHC-I and MHC-II as compared to monocytes, M1 macrophages, M2 macrophages, and MoDCs), b) are TLR2+/high and STING+/high (e.g., express a higher level of TLR2 and STING than M1 macrophages), and c) express a higher level of CD40 as compared to monocytes, M1 macrophages, M2 macrophages, and MoDCs. In some embodiments, the APCs a) do not express or express a lower level of CD141, XCR1, and CD103 as compared to cDC1, b) do not express or express a lower level of CD1c and SIRPα as compared to cDC2, c) express a lower level of CD303 as compared to pDC or M1 macrophages, d) express a lower level of CCR7, CCR2 and CD11c as compared to M1 macrophages. In some embodiments, the APCs have a size of less than about 15 μm (e.g., about 7-15 μm). In some embodiments, the APCs have a spindle or elongated shape, or multi-shaped. In some embodiments, the APCs adhere to the matrix substratum but can be easily dislodged by repeated pipetting or brief trypsinization (<2 min at 37° C.). In some embodiments, the APCs a) are CD80+/high and CD86+/high (e.g., express a higher level of CD80 and CD86 as compared to monocytes, M1 macrophages, M2 macrophages, and MoDCs). In some embodiments, the expression level of CD40 on the APCs are at least 5-fold, 10-fold, 20-fold, 50-fold, or 100-fold higher than that on monocytes, M1 macrophages, M2 macrophages, and MoDCs.

In some embodiments, there are provided a population of APCs (such as APCs derived from monocytes obtained from an individual (e.g., a healthy individual, a cancer patient or a virus infected patient), wherein the APCs a) are MHC-I+/high and MHC-II+/high (e.g., express a higher level of MHC-I and MHC-II as compared to monocytes, M1 macrophages, M2 macrophages, and MoDCs), b) express or express a high level of at least one of LOX1 and uPAR (e.g., a higher level of LOX1 and uPAR as compared to monocytes, M1 macrophages, M2 macrophages, and MoDCs), and c) express a higher level of CD40 as compared to monocytes, M1 macrophages, M2 macrophages, and MoDCs. In some embodiments, the APCs express a high level of at least one of TLR2 and STING (e.g., a higher level of TLR2 and STING than M1 macrophages). In some embodiments, the APCs a) do not express or express a lower level of CD141, XCR1, and CD103 as compared to cDC1, b) do not express or express a lower level of CD1c and SIRPα as compared to cDC2, c) express a lower level of CD303 as compared to pDC or M1 macrophages, d) express a lower level of CCR7, CCR2 and CD11c as compared to M1 macrophages. In some embodiments, the APCs have a size of less than about 15 μm (e.g., about 7-15 μm). In some embodiments, the APCs have a spindle or elongated shape, or multi-shaped. In some embodiments, the APCs adhere to the matrix substratum but can be easily dislodged by repeated pipetting or brief trypsinization (<2 min at 37° C.). In some embodiments, the APCs a) are CD80+/high and CD86+/high (e.g., express a higher level of CD80 and CD86 as compared to monocytes, M1 macrophages, M2 macrophages, and MoDCs). In some embodiments, the expression level of CD40 on the APCs are at least 5-fold, 10-fold, 20-fold, 50-fold, or 100-fold higher than that on monocytes, M1 macrophages, M2 macrophages, and MoDCs.

In some embodiments, there are provided a population of APCs (such as APCs derived from monocytes obtained from an individual (e.g., a healthy individual, a cancer patient or a virus infected patient), wherein the APCs a) are MHC-I+/high and MHC-II+/high (e.g., express a higher level of MHC-I and MHC-II as compared to monocytes, M1 macrophages, M2 macrophages, and MoDCs), b) express a high level of at least one of LOX1 and uPAR (e.g., a higher level of LOX1 and uPAR as compared to monocytes, M1 macrophages, M2 macrophages, and MoDCs), and c) b) are TLR2+/high and STING+/high (e.g., express a higher level of TLR2 and STING than M1 macrophages). In some embodiments, the APCs a) do not express or express a lower level of CD141, XCR1, and CD103 as compared to cDC1, b) do not express or express a lower level of CD1c and SIRPα as compared to cDC2, c) express a lower level of CD303 as compared to pDC or M1 macrophages, d) express a lower level of CCR7, CCR2 and CD11c as compared to M1 macrophages. In some embodiments, the APCs have a size of less than about 15 μm (e.g., about 7-15 μm). In some embodiments, the APCs have a spindle or elongated shape, or multi-shaped. In some embodiments, the APCs adhere to the matrix substratum but can be easily dislodged by repeated pipetting or brief trypsinization (<2 min at 37° C.). In some embodiments, the APCs a) are CD80+/high and CD86+/high (e.g., express a higher level of CD80 and CD86 as compared to monocytes, M1 macrophages, M2 macrophages, and MoDCs). In some embodiments, the expression level of CD40 on the APCs are at least 5-fold, 10-fold, 20-fold, 50-fold, or 100-fold higher than that on monocytes, M1 macrophages, M2 macrophages, and MoDCs.

In some embodiments, there are provided a population of APCs derived from monocytes obtained from an individual (e.g., a healthy individual, a cancer patient or a virus infected patient), wherein the APCs a) have a spindle or elongated shape, or are multi-shaped, b) express a higher or comparable level of MHC-I, MHC-II, CD80, CD86, CD40, OX40L, ICOSL, and/or CD70 as compared to mature dendritic cells (e.g., derived from monocytes after culture with LPS or TNFα), c) express a higher level of LOX1, uPAR, CD40, TLR2, IL-3R. TREM2, C3AR, IL-3R and/or PD-L1 as compared to mature dendritic cells (e.g., derived from monocytes after culture with LPS or TNFα), monocytes, M1 or M2 macrophages, d) express a lower level of DEC205, SIRPα, and/or CD11c as compared to mature dendritic cells (e.g., derived from monocytes after culture with LPS or TNFα) or M1 macrophages, c) express a lower level of CD11c and CD303 as compared to mature dendritic cells (e.g., derived from monocytes after culture with LPS or TNFα) or M1 macrophages, f) express a lower level of CD26, CCR7 and/or CCR2 than M1 macrophages, and/or g) express a higher level of Sting and/or TLR2, optionally TLR3, and/or TLR8 as compared to M1 macrophages.

In some embodiments, the APCs express a higher level of CCL3L1, CXCL8, IL-6, IL1B, CCL2, CXCL1, CXCL2, CXCL3, CCL7, C3AR1, SLC16A6, CXCL5, and/or SERPINB2 as compared to mature dendritic cells (e.g., derived from monocytes after culture with LPS). In some embodiments, the APCs express a lower level of CCL22, CSTB, LIPA, CCL17, CCL13, APOE, FABP4. CD1B, FN1, CD1c, CDIA, and/or PTGDS as compared to mature dendritic cells (e.g., derived from monocytes after culture with LPS). In some embodiments, the APCs express a higher level of CXCL8, IL-6, NAMPT, CXCL1, CCL6, CXCL3, CCL18, PELI1, CXCL5, SLC16A6, and/or SERPINB2 as compared to M1 macrophages. In some embodiments, the APCs express a lower level of CXCL10, MMP9, LIPA, S100A4, CCL22, IL12B, APOE, CRABP2, PTGDS, and/or FN1 as compared to M1 macrophages. In some embodiments, the APCs express a higher level of C3AR1, olr1, TLR2 and PLAUR as compared to mature dendritic cells (e.g., derived from monocytes after culture with LPS or TNFα), M1/M2 macrophages, and/or monocytes. In some embodiments, the APCs express a lower level of DC specific antigens such as CD11c, CD1a, CD1c, Batf3 as compared to mature dendritic cells (e.g., derived from monocytes after culture with LPS or TNFα). In some embodiments, the APCs express a lower level of macrophage specific antigens (e.g., CD68) as compared to M1/M2 macrophages. In some embodiments, the APCs express a lower level of monocyte specific antigens (e.g., CCR2, CXCR1, CD14). In some embodiments, the APC express a lower level of CD31 as compared to monocytes.

In some embodiments, there are provided a population of APCs derived from monocytes obtained from an individual (e.g., a cancer patient or a virus infected patient), wherein the APCs a) express a high level of one or more antigen presentation molecule, wherein the antigen presentation molecule is selected from the group consisting of: MHCI, MHCII, CD86, CD80, OX40L, ICAML, ICOSL, and CD40, and/or b) a low level of an inhibitory signaling molecule, wherein the inhibitory signaling molecule is selected from the group consisting of: TGFβR, SIRPα, LILRB (LILRB1 and/or LILRB2) and Siglec 10. In some embodiments, the monocytes exhibit a lower expression level of M-CSFR, GM-CSFR. IL-6R, IL-10R, and/or IL-4R (e.g., at least about 10%, 20%, 30%, 40%, 50%, or 60% lower) at the time when they are obtained from the individual as compared to the monocytes obtained from a reference individual (e.g., a healthy individual). In some embodiments, at least about 20%, 30%, 40%, 50%, 60%, 70%, 80%, or 90% of the APCs have a dendritic cell morphology.

In some embodiments, there are provided a population of APCs derived from monocytes obtained from an individual (e.g., a cancer patient or a virus infected patient), wherein the APCs a) express a high level of MHC I, MHC II, CD80, CD40, OX40L (e.g., at least 20%, 30%, 40%, 50%, 60%, 70%, 80%, 90%, or 100% higher than the level of corresponding molecule on monocytes obtained from same individual and cultured with GM-CSF and M-CSF for about 2 days); b) produce a high level of IL-12, type I and/or type II IFN, TNFα, IL-1, and IL-6 (e.g., at least 20%, 30%, 40%, 50%, 60%, 70%, 80%, 90%, or 100% higher than the level of corresponding molecule on monocytes obtained from same individual and cultured with GM-CSF and M-CSF for about 2 days); c) express a low level of TGFβR, SIRPα, LILRB (LILRB1 and/or LILRB2) and Siglec 10 (e.g., at least about 10%, 20%, 30%, 40%, or 50% lower than the level of corresponding molecule on monocytes obtained from same individual and cultured with GM-CSF and M-CSF for about 2 days); and/or d) at least about 20%, 30%, 40%, 50%, 60%, 70%, 80%, or 90% of the APCs have a dendritic cell morphology.

The APCs described herein in some embodiments are derived from monocytes obtained from an individual (e.g., a cancer patient or a virus infected patient). In some embodiments, the monocytes exhibit a lower expression level of M-CSFR (e.g., at least about 10%, 20%, 30%, 40%, 50%, or 60% lower) at the time when they are obtained from the individual as compared to the monocytes obtained from a reference individual (e.g., a healthy individual). In some embodiments, the monocytes exhibit a lower expression level of GM-CSFR (e.g., at least about 10%, 20%, 30%, 40%, 50%, or 60% lower) at the time when they are obtained from the individual as compared to the monocytes obtained from a reference individual (e.g., a healthy individual). In some embodiments, the monocytes exhibit a lower expression level of both M-CSFR and G-CSFR (e.g., at least about 10%, 20%, 30%, 40%, 50%, or 60% lower) at the time when they are obtained from the individual as compared to the monocytes obtained from a reference individual (e.g., a healthy individual).

In some embodiments, the monocytes exhibit a lower expression level of IL-10R (e.g., at least about 10%, 20%, 30%, 40%, 50%, or 60% lower) at the time when they are obtained from the individual as compared to the monocytes obtained from a reference individual (e.g., a healthy individual). In some embodiments, the monocytes exhibit a lower expression level of IL-6R (e.g., at least about 10%, 20%, 30%, 40%, 50%, or 60% lower) at the time when they are obtained from the individual as compared to the monocytes obtained from a reference individual (e.g., a healthy individual). In some embodiments, the monocytes exhibit a lower expression level of IL-4R (e.g., at least about 10%, 20%, 30%, 40%, 50%, or 60% lower) at the time when they are obtained from the individual as compared to the monocytes obtained from a reference individual (e.g., a healthy individual). In some embodiments, the monocytes exhibit a lower expression level of IFNGR (e.g., at least about 10%, 20%, 30%, 40%, 50%, or 60% lower) at the time when they are obtained from the individual as compared to the monocytes obtained from a reference individual (e.g., a healthy individual)

In some embodiments, the APCs express a high level of one or more (e.g., two, three, four, five, six, seven, or eight) antigen presentation molecule, wherein the antigen presentation molecule is selected from the group consisting of: MHCI, MHCII, CD86, CD80, OX40L. ICAML, ICOSL, and CD40, optionally wherein the APCs are produced from monocytes (such as any monocytes described herein, e.g., monocytes obtained from a cancer patient) in a cell culture. In some embodiments, the APC express a high level of MHC I, MHC II, CD86, CD80, CD40, and/or OX40L.

In some embodiments, the APCs express a high level of one or more (e.g., two, three, four, five, six, seven, or eight) antigen presentation molecule, wherein the antigen presentation molecule is selected from the group consisting of: MHCI, MHCII, CD86, CD80, OX40L, ICAML, ICOSL, and CD40 when the level of one or more antigen presentation molecule selected from the group consisting of: MHCI, MHCII, CD86, CD80, OX40L, ICAML, ICOSL, and CD40 is at least about 20%, 30%, 40%, 50%, 60%, 70%, 80%, 90%, or 100% higher than the level of corresponding molecule on monocytes obtained from same individual and cultured with GM-CSF and M-CSF (e.g., for about 2 days).

In some embodiments, the APCs express a high level of one or more (e.g., two, three, four, five, six, seven, or eight) antigen presentation molecule, wherein the antigen presentation molecule is selected from the group consisting of: MHCI, MHCII, CD86, CD80, OX40L, ICAML, ICOSL, and CD40 when the level of one or more (e.g., two, three, four, five, six, seven, or eight) antigen presentation molecule selected from the group consisting of: MHCI, MHCII, CD86, CD80, OX40L, ICAML, ICOSL, and CD40 is at least about 20%, 30%, 40%, 50%, 60%, 70%, 80%, 90%, or 100% higher than the level of corresponding molecule on dendritic cells obtained from a healthy human and cultured with GM-CSF and IL-4 for about 5 days.

In some embodiments, the APC express a high level of MHC I, MHC II, CD86, CD80, CD40, and/or OX40L.

In some embodiments, the APCs produce a high level of one or more (e.g., at least two, three, four, five, or six) cytokines selected from the group consisting of IL-12, type I and/or type II IFN, TNFα, IL-1, and IL-6, when the level of one or more (e.g., at least two, three, four, five, or six) cytokines selected from the group consisting of IL-12, type I and/or type II IFN, TNFα, IL-1, and IL-6 is at least about 20%, 30%, 40%, 50%, 60%, 70%, 80%, 90%, or 100% higher than the level of corresponding cytokine on monocytes obtained from same individual and cultured with GM-CSF and M-CSF (e.g., for about 2 days).

In some embodiments, the APCs produce a high level of PD-L1, when the level of PD-L1 is at least about 50%, 60%, 70%, 80%, 90%, 100%, 125%, 150%, 175% or 200% higher than the level of PD-L1 on monocytes they are derived from when obtained from the individual.

In some embodiments, the APCs express a low level of an inhibitory signaling molecule, wherein the inhibitory signaling molecule is selected from the group consisting of: TGFβR, SIRPα, LILRB (LILRB1 and/or LILRB2) and Siglec 10. In some embodiments, the APCs express a low level of SIRPα.

In some embodiments, the APCs express a low level of an inhibitory signaling molecule when the level of one or more antigen presentation molecule selected from the group consisting of: TGFβR, SIRPα, LILRB (LILRB1 and/or LILRB2) and Siglec 10 is at least about 10%, 20%, 30%, 40%, or 50% lower than the level of corresponding molecule on monocytes obtained from same individual and cultured with GM-CSF and M-CSF (e.g., for about 2 days).

In some embodiments, the APCs express a low level of an inhibitory signaling molecule when the level of one or more antigen presentation molecule selected from the group consisting of: TGFβR, SIRPα, LILRB (LILRB1 and/or LILRB2) and Siglec 10 is at least about 10%, 20%, 30%, 40%, or 50% lower than the level of corresponding molecule on dendritic cells obtained from a healthy human and cultured with GM-CSF and IL-4 for about 5 days.

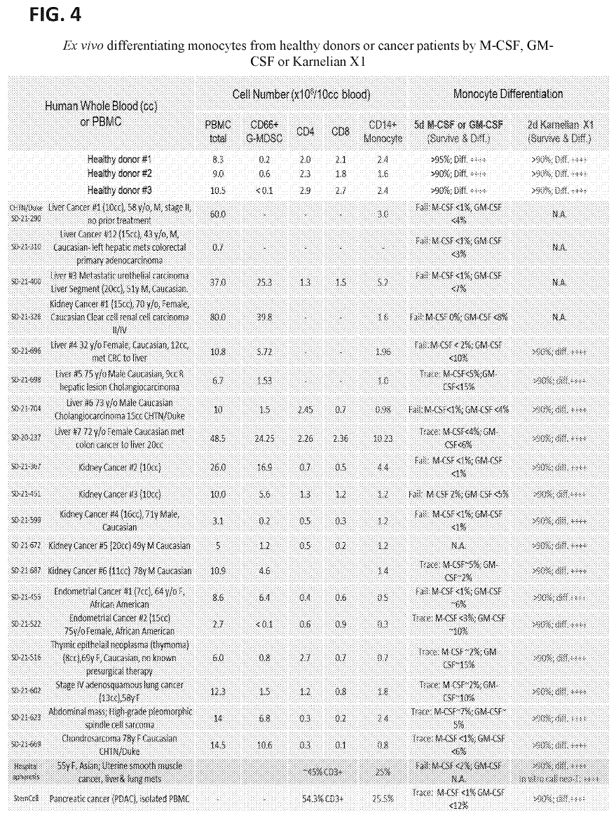

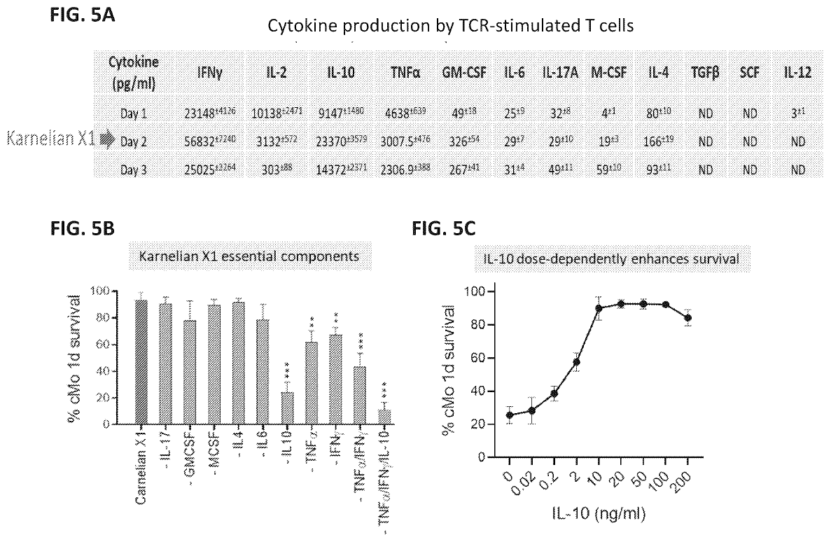

In some embodiments, the APCs have a morphology substantially the same as shown in FIG. 4F (right).

In some embodiments, at least about 20%, 30%, 40%, 50%, 60%, 70%, 80%, or 90% of the APCs have a dendritic cell morphology.

In some embodiments, the APCs comprise one or more tumor-associated antigen peptides, e.g., neoantigen peptides (such as any of those discussed herein).

In some embodiments, the APCs comprise one or more virus-associated antigen peptides.

In some embodiments, the APCs are capable of promote proliferation of immune cells (e.g., T cells, e.g., CD4 T cells and/or CD8 T cells) upon incubation with the immune cells. In some embodiments, the APCs promote proliferation of T cells for at least about 5-fold, 10-fold, 15-fold or 20-fold in a culture comprising IL-2, IL-7 and IL-15. In some embodiments, the incubation is no longer than about 24 hours, 22 hours, 20 hours or 18 hours. In some embodiments, the APCs present one or more disease associated peptides (e.g., tumor peptides) to immune cells.

In some embodiments, there is provided a composition (e.g., a culture) comprising the APCs described herein. In some embodiments, the APCs present one or more disease associated peptides (e.g., tumor peptides) to immune cells.

### Tumor-Associated Antigen Peptides

Various approaches are available to identify tumor-associated antigen peptides.

One approach often employed to identify the peptides recognized by such CTL is expression cloning, which consists in isolating the peptide-encoding gene by transfecting a library of tumoral cDNA and testing the transfected cells for their ability to activate the CTL clone. Fragments of the identified gene can then be transfected to define the region encoding the antigenic peptide, and finally candidate peptides bearing adequate HLA-binding motifs are tested for their ability to sensitize target cells to lysis by the CTL. This approach was successfully used to identify a large number of antigenic peptides.

Nowadays, tumor-associated antigenic peptides are often identified using the “reverse immunology” approach, which consists in selecting peptides with adequate HLA-binding motifs inside a protein of interest, such as proteins encoded by mutated oncogenes or genes that are either selectively expressed or overexpressed by tumors. Candidate peptides are synthesized and tested for HLA binding in vitro. The most efficient binders are pulsed onto antigen-presenting cells, which are used to stimulate T lymphocytes in vitro, in order to derive CTL lines or clones that recognize peptide-pulsed target cells. A drawback of this approach is that the identified peptides might not be processed efficiently by tumors. It is therefore essential to verify that the CTL do recognize tumor cells that naturally express the peptide-encoding gene. Additionally, one should test transfectants that express normal levels of the gene or cells where expression of the gene has been knocked down using si or shRNAs.

A third approach to antigen identification is based on the elution of antigenic peptides from MHC class I molecules immunopurified from the surface of tumor cells. The direct identification by mass spectrometry of the sequence of the eluted peptides is technically demanding but proved useful to identify or to confirm the relevance of peptides that have undergone posttranslational modifications such as serine/threonine phosphorylation, glycosylation-dependent asparagine deamidation, or peptide splicing.

A large number of antigenic peptides recognized by antitumor CTL have been identified using these various approaches. These antigens are conveniently classified according to the expression pattern of the parent gene. A regularly updated database of those antigenic peptides effectively presented by tumor cells can be found on the http://www.cancerimmunity.org/website. See Vigneron, Biomed Res Int. 2015; 2015:948501.

The present application provides APCs produced by any of the methods described here.

### Neoantigen Peptides

Various methods are available to detect and screen neoantigens. Sandwich immunoassays in the miniaturized system could successfully identify tumor antigens in sera samples extracted from patients. See e.g., Pollard et al., Proteomics Clin. Appl. 1 934-952 (2007); Yang et al., Biosens. Bioelectron. 40 385-392 (2013). Another tool named Serologic Protcome analysis (SERPA) or 2-D western blots, consists of the isoelectric focusing (IEF) gel run in the first dimension and SDS-PAGE gel run in the second dimension. SERPA separates the proteins in the gel by their isoelectric point (IP) and molecular mass and then transfers the proteins from the gel to a carrier membrane to screen antibodies. Finally, the antigenic protein spots can be identified by MS. See e.g., Tjalsma et al., Proteomics Clin. Appl. 2 167-180 (2008). This approach has been used to identify antigens in different tumor types. Serological analysis of recombinant cDNA expression libraries (SEREX), which combines serological analysis with antigen cloning techniques, is a widely used technique to explore tumors' antigen repertoire. SEREX first construct a cDNA library from cancer cell lines or fresh tumor samples, then screen the cDNA library with autologous sera of cancer patients, and finally sequence the immune-reactive clones. SEREX have identified a variety of tumor antigens including CTAs, differentiation antigens, mutational antigens, splice-variant antigens and overexpressed antigens. See e.g., Chen et al., Proc. Natl. Acad. Sci. U.S.A. 94 1914-1918 (1997). Furthermore, other methods such as Multiple Affinity Protein Profiling (MAPPing) and nanoplasmonic biosensor have also been developed to identify tumor antigens. See e.g., Lec et al., Biosens. Bioelectron. 74 341-346 (2015).

In some embodiments, the one or more neoantigenic peptides described herein are obtained from a neoantigenic database (such as any of the neoantigenic databases described herein). For example, Tan et al constructed a manually curated database (“dbPepNeo”) for human tumor neoantigen peptides based upon the four criterias as below: (i) peptides were isolated from human tumor tissues or cell lines, (ii) peptides contained non-synonymous mutations in amino acid sequence, (iii) Peptides can be bound by HLA-I molecules, (iv) Peptides can induce CD8+ T cell responses. See Tan et al., Database (Oxford). 2020 Jan. 1; 2020: baaa004. Xia et al constructed another database, NEPdb, which provides pan-cancer level predicted HLA-I neoepitopes derived from 16,745 shared cancer somatic mutations, using state-of-the-art predictors. See Xia et al., Front Immunol. 2021; 12:644637. Wu et al. developed a comprehensive tumor-specific neoantigen database (TSNAdb v1.0), based on pan-cancer immunogenomic analyses of somatic mutation data and human leukocyte antigen (HLA) allele information for 16 tumor types with 7748 tumor samples from The Cancer Genome Atlas (TCGA) and The Cancer Immunome Atlas (TCIA). Sec Wu et al., enomics Proteomics Bioinformatics. 2018 August; 16 (4): 276-282.

In some embodiments, the one or more neoantigenic peptides are obtained from analyzing the biological information of the individual (such as a patient who had a cancer). In some embodiments, the neoantigenic peptides are obtained from a computational analysis of a cancer patient's tumor genome. See e.g., Roudko et al. Front Immunol. 2020; 11:27. In some embodiments, the neoantigenic peptides are obtained from a computational analysis of a cancer patient's transcriptome. See e.g., Caushi et al., Nature. 2021 August; 596 (7870): 126-132. In some embodiments, the neoantigenic peptides are obtained from a computational analysis of a cancer patient's proteome. See e.g., Wen et al. Nat Commun. 2020 Apr. 9; 11 (1): 1759.

In some embodiments, the neoantigenic peptides are selected based upon patient data. In some embodiments, the patient data is derived from data from a group of patients having a particular type of cancer (e.g., any of the cancers described here). In some embodiments, the patient data is derived from data from a group of patients having any cancer. In some embodiments, the group of patients are from the same sex (e.g., male or female). In some embodiments, the group of patients are from the same ethnicity. In some embodiments, the group of patients bear one or more biomarkers (e.g., an aberration in a particular gene, e.g., KRAS, e.g., PTEN).

In some embodiments, the one or more neoantigenic peptides are derived from any polypeptide known to or have been found to contain a tumor specific mutation. Suitable polypeptides from which the neoantigenic peptides can be derived can be found for example in various databases available in the field (e.g., COSMIC database). These databases curate comprehensive information on somatic mutations in human cancer. In some embodiments, the peptide contains a tumor specific mutation. In some embodiments, the tumor specific mutation is a driver mutation for a particular cancer type.

In some embodiments, the tumor-associated peptides (e.g., neoantigen peptides) are synthetic peptides. In some embodiments, the neoantigenic peptides are obtained by exome high throughput sequencing and prescreened with epitope prediction algorithms.

In some embodiments, the one or more neoantigenic peptides are selected based upon its binding affinity to an MHC molecule (e.g., an MHC I molecule and/or an MHC II molecule). In some embodiments, the neoantigenic peptide has a binding affinity that is less than 5000 nM (e.g., less than 500 nM, less than 250 nM, less than 100 nM or less than 50 nM) (IC50) to an MHC molecule. In some embodiments, the neoantigenic peptide has a binding affinity of about 500 nM to 5000 nM (IC50) to an MHC molecule. In some embodiments, the neoantigenic peptide has a binding affinity that is less than 500 nM (IC50) to an MHC molecule. In some embodiments, the neoantigenic peptide has a binding affinity of about 250 nM to 500 nM IC50 to an MHC molecule. In some embodiments, the neoantigenic peptide has a binding affinity that is less than 250 nM (IC50) to an MHC molecule. In some embodiments, the neoantigenic peptide has a binding affinity that is less than 100 nM (IC50) to an MHC molecule. In some embodiments, the neoantigenic peptide has a binding affinity of about 50 nM to 500 nM IC50 to an MHC molecule. In some embodiments, the neoantigenic peptide has a binding affinity that is less than 50 nM (IC50) to an MHC molecule. In some embodiments, the neoantigenic peptide has a binding affinity of about 1 nM to 50 nM IC50 to an MHC molecule.

In some embodiments, a plurality of tumor-associated peptides (e.g., neoantigen peptides) are prepared from a surgical resection of tumor tissue or a biopsy extract thereof.

In some embodiments, a plurality of tumor-associated peptides (e.g., neoantigen peptides) are prepared from a mixture of tumor cells or extract thereof isolated from tumor tissue or biopsy.

In some embodiments, a plurality of tumor-associated peptides (e.g., neoantigen peptides) are prepared from a mixture of isolated tumor-associated peptides (e.g., neoantigen peptides).

In some embodiments, the tumor tissue or cell described above is a fresh tumor tissue or cells. In some embodiments, the tumor tissue or cell is obtained from a frozen sample.

In some embodiments, the tumor tissue or cells have been subjected to an induction of immunogenic cell death (e.g., freeze-thaw to lysis tumor cells, high dose UV irradiation, X-ray radiation).

In some embodiments, the tumor tissue or cells have been subjected to a radiation treatment.

### IV. Methods of Activating Immune Cells and Activated Immune Cell Compositions

The present application also provides methods of activating a population of immune cells. In some embodiments, the methods comprise co-culturing the population of immune cells with the population of the APCs described herein, wherein the APCs are pre-loaded with one or more antigen peptides, (e.g., tumor peptides, e.g., tumor-associated peptides, e.g., neoantigen peptides).

In some embodiments, there is provided a method of activating a population of immune cells (e.g., T cells, e.g., TIL cells) obtained from an individual (e.g., a cancer patients), comprising co-culturing the population of immune cells with the population of the APCs (e.g., the APCs described herein), thereby producing a population of activated immune cells, wherein the APCs are pre-loaded with one or more tumor peptides. In some embodiments, the APCs are derived from monocytes obtained from the same individual. In some embodiments, the APCs have been pre-incubated with tumor antigens (e.g., free-thaw tumor cell/debris). In some embodiments, the pre-incubation is about 3-10 hours (e.g., about 6 hours). In some embodiments, the ratio of APCs and the immune cells (e.g., T cells, e.g., TIL cells) during co-culturing is about 10:1 to about 1:10 (e.g., about 5:1 to about 1:5, about 2:1 to about 1:2, about 1:1). In some embodiments, the APCs and the immune cells are co-cultured for about 2-20 days. In some embodiments, IL-2, IL-7 and/or IL-15 are supplemented to the co-culture (e.g., are supplemented about at least 2 or 3 days after the co-culture). In some embodiments, the activated immune cells comprise at least 5-, 10-, or 20-fold (e.g., 50-100-fold) more cells than the immune cells prior to the co-culture. In some embodiments, the activated immune cells are subject to the activation via co-culture with APC described herein for at least two, three, four, or five rounds. In some embodiments, the activated immune cells do not exhibit an exhaustive feature (e.g., senescence) after two, three or four consecutive rounds (e.g., about 6-10 days each round) of activation that involves the co-culture described herein. In some embodiments, the co-culture does not involve use of an anti-CD3 antibody and/or an anti-CD28 antibody at least some of the rounds (e.g., anti-CD3 and anti-CD28 antibodies are only used in the first round but not the later rounds).

In some embodiments, the method comprises contacting the APCs with a composition comprising a plurality of tumor-associated peptides (e.g., neoantigen peptides). In some embodiments, the APCs are allowed to be in contact with the composition comprising a plurality of tumor-associated peptides (e.g., neoantigen peptides) for about 4 to about 24 hours.

In some embodiments, the APCs have been pre-incubated with the composition comprising a plurality of tumor-associated peptides (e.g., neoantigen peptides).

In some embodiments, the composition comprising a plurality of tumor-associated peptides (e.g., neoantigen peptides) is a surgical resection of tumor tissue or a biopsy extract thereof.

In some embodiments, the composition comprising a plurality of tumor-associated peptides (e.g., neoantigen peptides) is a mixture of tumor cells or extract thereof isolated from tumor tissue or biopsy.

In some embodiments, the composition comprising a plurality of tumor-associated peptides (e.g., neoantigen peptides) is a mixture of isolated tumor-associated peptides (e.g., neoantigen peptides).

In some embodiments, the tumor tissue or cell is a fresh tumor tissue or cells. In some embodiments, the tumor tissue or cell is obtained from a frozen sample.

In some embodiments, the tumor tissue or cells have been subjected to an apoptosis induction.

In some embodiments, the tumor tissue or cells have been subjected to a radiation treatment.

In some embodiment, the population of immune cells and the APCs are derived from the same individual.

In some embodiments, the population of immune cells and the antigen presenting cells are not derived from the same individual.

The present application also provides activated immune cells (e.g., T cells) produced by any of the methods described here.

### Tumor-Associated Peptides Loading

In some embodiments, the methods described herein further comprise contacting APCs with a plurality of tumor-associated peptides (e.g., neoantigen peptides). In some embodiments, the plurality of tumor-associated peptides (e.g., neoantigen peptides) have more than about any of 1, 2, 3, 4, 5, 6, 7, 8, 9, 10, 11, 12, 13, 14, 15, 16, 17, 18, 19, 20, 22, 25, 30, 40, or 50 tumor-associated peptides (e.g., neoantigen peptides). In some embodiments, the APCs are allowed to be in contact with the composition comprising a plurality of tumor-associated peptides (e.g., neoantigen peptides) for about 4 to about 24 hours.

In some embodiments, the APCs have been pre-incubated with the composition comprising a plurality of tumor-associated peptides (e.g., neoantigen peptides) prior to be used in the methods of activating immune cells described herein.

An exemplary embodiment of the contacting of a population of APCs with a plurality of tumor-associated peptides (e.g., neoantigen peptides) comprises pulsing the plurality of tumor-associated peptides (e.g., neoantigen peptides) into the population of APCs. As known in the art, pulsing refers to a process of mixing cells, such as APCs, with a solution containing tumor-associated peptides (e.g., neoantigen peptides), and optionally subsequently removing the tumor-associated peptides (e.g., neoantigen peptides) from the mixture. The population of APCs may be contacted with a plurality of tumor-associated peptides (e.g., neoantigen peptides) for seconds, minutes, or hours, such as about any of 30 seconds, 1 minute, 5 minutes, 10 minutes, 15 minutes, 20 minutes, 30 minutes, 1 hour, 5 hours, 10 hours, 12 hours, 14 hours, 16 hours, 18 hours, 20 hours, 22 hours, 1 day, 2 days, 3 days, 4 days, 5 days, 6 days, one week, 10 days, or more. The concentration of each neoantigen peptide used in the contacting step may be about any of 0.1, 0.5, 1, 2, 3, 5, or 10 μg/mL. In some embodiments, the concentration of the tumor-associated peptides (e.g., neoantigen peptides) is about 0.1-200 μg/mL, including for example about any of 0.1-0.5, 0.5-1, 1-10, 10-50, 50-100, 100-150, or 150-200 μg/mL.

In some embodiments, the population of APCs is contacted with the plurality of tumor-associated peptides (e.g., neoantigen peptides) in the presence of a composition that facilitates the uptake of the plurality of tumor-associated peptides (e.g., neoantigen peptides) by the APCs. In some embodiments, compounds, materials, or compositions may be included in a solution of the plurality of tumor-associated peptides (e.g., neoantigen peptides) to facilitate peptide uptake by the APCs. Compounds, materials, or compositions that facilitate the uptake of the plurality of tumor-associated peptides (e.g., neoantigen peptides) by the APCs include, but are not limited to, lipid molecules and peptides with multiple positively charged amino acids. In some embodiments, more than about any of 50%, 60%, 70%, 80%, 90%, or 95% of the tumor-associated peptides (e.g., neoantigen peptides) are uptaken by the population of APCs. In some embodiments, more than about any of 50%, 60%, 70%, 80%, 90%, or 95% of the APCs in the population uptake at least one tumor antigen peptide.

### Immune Cells

The immune cells described herein can be any type of immune cells that interact with APCs and can be activated by APCs, and then exert their desired functions. Exemplary immune cells include T cells.

T cells, or T lymphocytes, play a central role in cell-mediated immunity. Each clone of activated T cells express a distinct T-cell receptor (TCR) on the surface, which is responsible for recognizing antigens bound to MHC molecules on APCs and on target cells (such as cancer cells). T cells are subdivided into several types, each expressing a unique combination of surface proteins and each having a distinct function.

Cytotoxic T cells (TC) participate in the immune response to and destruction of tumor cells and other infected cells, such as virus-infected cells. Generally, TC cells function by recognizing a class I MHC presented antigen on an APC or any target cell. Stimulation of the TCR, along with a co-stimulator (for example CD28 on the T cell binding to B7 on the APC, or stimulation by a helper T cell), results in activation of the TC cell. The activated TC cell can then proliferate and release cytotoxins, thereby destroying the APC, or a target cell (such as a cancer cell). Mature TC cells generally express surface proteins CD3 and CD8. Cytotoxic T cells belong to CD3+CD8+ T cells.

Helper T cells (TH) are T cells that help the activity of other immune cells by releasing T cell cytokines, which can regulate or suppress immune responses, induce cytotoxic T cells, and maximize cell killing activities of macrophages. Generally, TH cells function by recognizing a class II MHC presented antigen on an APC. Mature TH cells express the surface proteins CD3 and CD4. Helper T cells belong to CD3+CD4+ T cells.

Regulatory T cells (T_{REG} cells) generally modulate the immune system by promoting tolerance for self-antigens, thereby limiting autoimmune activity. In cancer immunotherapy, T_{REG} contributes to escape of the cancer cells from the immune response. T_{REG} cells generally express CD3, CD4, CD7, CD25, CTLA4, GITR, GARP, FOXP3, and/or LAP. CD4+CD25+Foxp3+ T cells are one class of T_{REG} cells.

Memory T cells (Tm) are T cells that have previously encountered and responded to their specific antigens, or T cells that differentiated from activated T cells. Although tumor specific Tms constitutes a small proportion of the total T cell amount, they serve critical functions in surveillance of tumor cells during a person's entire lifespan. If tumor specific Tms encounter tumor cells expressing their specific tumor antigens, the Tms are immediately activated and clonally expanded. The activated and expanded T cells differentiate into effector T cells to kill tumor cells with high efficiency. Memory T cells are important for establishing and maintaining long-term tumor antigen specific responses of T cells.

Typically, an antigen for T cells is a protein molecule or a linear fragment of a protein molecule that can be recognized by a T-cell receptor (TCR) to elicit specific T cell response. The antigen can be derived from a foreign source such as a virally encoded protein, or an endogenous source such as a protein expressed on the cell surface. The minimal fragment of an antigen that is directly involved in interaction with a particular TCR is known as an epitope. Multiple epitopes can exist in a single antigen, wherein each epitope is recognized by a distinct TCR encoded by a particular clone of T cells.

In order to be recognized by a TCR, an antigen peptide or antigen fragment is processed into an epitope by an APC (such as a dendritic cell), and then bound in an extended conformation inside a Major Histocompatibility (MHC) molecule to form an MHC-peptide complex on the surface of an APC (such as a dendritic cell). MHC molecules are also known as human leukocyte antigens (HLA). The MHC provides an enlarged binding surface for strong association between TCR and epitope, while a combination of unique amino acid residues within the epitope ensures specificity of interaction between TCR and the epitope.

The human MHC molecules are classified into two types-MHC class I and MHC class II-based on their structural features, especially the length of epitopes bound inside the corresponding MHC complexes. MHC-I epitopes are epitopes bound to and represented by an MHC class I molecule. MHC-II epitopes are epitopes bound to and represented by an MHC class II molecule. MHC-I epitopes are typically about 8 to about 11 amino acids long, whereas MHC-II epitopes are about 13 to about 17 amino acids long. Due to genetic polymorphism, various subtypes exist for both MHC class I and MHC class II molecules among the human population. T cell response to a specific antigen peptide presented by an MHC class I or MHC class II molecule on an APC is known as MHC-restricted T cell response.

In some embodiments, the immune cells are selected from the group consisting of PBMC, tumor infiltrating T cells (TIL), and T cells (e.g., CD4 T cells and/or CD8 T cells). In some embodiments, the immune cells are PBMC.

In some embodiments, the immune cells are tumor infiltrating T cells (TIL).

In some embodiments, the immune cells are CD4 T cells and/or CD8 T cells.

In some embodiments, the immune cells and the APCs are derived from the same individual. In some embodiments, the immune cells and the APCs are derived from different individuals.

### Preparation of Activated Immune Cells (e.g., Activated T Cells)

Methods described herein comprise co-culturing a population of immune cells (e.g., T cells) with a population of APCs described herein loaded with a plurality of tumor-associated peptides (e.g., neoantigen peptides).

In some embodiments, the co-culturing was carried out for at least 24 hours. In some embodiments, the co-culturing was carried out for at least about 1-5 days (e.g., about 1-3 days). In some embodiments, the population of immune cells (e.g., T cells) and the population of APCs loaded with the plurality of tumor-associated peptides (e.g., neoantigen peptides) are co-cultured for about any of 2, 4, 6, 8, 10, 12, 14, 16, 18, 20, 22, 24, 26, 28, or 30 days. In some embodiments, the population of immune cells (e.g., T cells) is co-cultured with the population of APCs loaded with the plurality of tumor-associated peptides (e.g., neoantigen peptides) for about 14 to about 21 days. In some embodiments, the population of immune cells (e.g., T cells) is co-cultured with the population of APCs loaded with the plurality of tumor-associated peptides (e.g., neoantigen peptides) for about 14 days.

The population of immune cells (e.g., T cells) used in any embodiment of the methods described herein may be derived from a variety of sources. A convenient source of the immune cells is from the PBMCs of the human peripheral blood. For example, the population of T cells may be isolated from the PBMCs, or alternatively, a population of PBMCs enriched with T cells (such as by addition of T cell specific antibodies and cytokines) can be used in the co-culture. In some embodiments, the population of T cells used in the co-culture is obtained from the non-adherent fraction of peripheral blood mononuclear cells (PBMCs). In some embodiments, the PBMCs are obtained by density gradient centrifugation of a sample of peripheral blood. In some embodiments, the population of activated T cells is prepared by obtaining a population of non-adherent PBMCs, and co-culturing the population of non-adherent PBMCs with a population of APCs loaded with a plurality of tumor-associated peptides (e.g., neoantigen peptides) (such as in the presence of at least one cytokine (such as IL-2) and an anti-CD3 antibody).

The co-culture may further include cytokines and other compounds to facilitate activation, maturation, and/or proliferation of the T cells, as well as to prime T cells for later differentiation into e.g., memory T cells. Exemplary cytokines that may be used in this step include, but are not limited to, IL-7, IL-15, IL-21 and the like. Certain cytokines may help suppress the percentage of T_{REG} in the population of activated T cells in the co-culture. For example, in some embodiments, a high dose (such as about any of 200, 300, 400, 500, 600, 700, 800, 900, 1000, 1200, or 1500 U/ml) of a cytokine (such as IL-2) is used to co-culture the population of T cells and the population of dendritic cells loaded with the plurality of tumor antigen peptides to obtain a population of activated T cells with a low percentage of T_{REG} cells.

In some embodiments, the methods of activating immune cells comprise co-culture of the immune cells (e.g., the T cells) and APC populations for more than one round (e.g., two, three or four rounds). In some embodiments, each round takes about 6-8 days. In some embodiments, the first, second, third and/or fourth round do not involve the addition of an anti-CD3 antibody and/or an anti-CD28 antibody. In some embodiment, the immune cells (e.g., the T cells) show non-exhaustive feature after two, three or four rounds of co-culture. In some embodiments, the immune cells (e.g., T cells) are capable of expand about 50-100 fold (e.g., at least 50-fold) after each round of culture. In some embodiments, each round takes about 5-10 days or 6-8 days. In some embodiments, the number of the immune cells (e.g., T cells) after three or four rounds of co-culture reaches about 10¹⁰.

In some embodiments, the methods of activating immune cells described herein further comprise expanding the population of immune cells following the co-culturing step. In some embodiments, expanding the population of immune cells comprises contacting the immune cells with a cytokine selected from the group consisting of IL-2, IL-7, and IL-15, optionally for about 2 to about 10 days. In some embodiments, the co-culture is in the presence of an anti-CD3 antibody and a plurality of cytokines, such as IL-2, IL-7, IL-15, IL-21 or any combination thereof.

The present application also provides populations of activated immune cells obtained by the methods described in this section.

### V. Methods of Treatment

The present application also provides methods of treating a disease or condition (e.g., a cancer, e.g., a virus infection) in a patient, comprising administering to the patient a population of APCs and/or activated immune cells obtained by the methods described above.

In some embodiments, there is provided a method of treating a disease or condition (e.g., a cancer, e.g., a virus infection) in a patient, comprising administering to the patient a population of APCs (such as any of those described in Section III, or produced according to methods described in Section II).

In some embodiments, there is provided a method of treating a disease or condition (e.g., a cancer, e.g., a virus infection) in a patient, comprising administering to the patient a population of antigen presenting cells (APCs), wherein the APCs are derived from monocytes obtained from an individual (e.g., a cancer patient or a virus infected patient), wherein the APCs a) express a high level of one or more antigen presentation molecule, wherein the antigen presentation molecule is selected from the group consisting of: MHCI, MHCII, CD86, CD80, OX40L, ICAML, ICOSL, and CD40, and/or b) a low level of an inhibitory signaling molecule, wherein the inhibitory signaling molecule is selected from the group consisting of: TGFβR, SIRPα, LILRB (LILRB1 and/or LILRB2) and Siglec 10. In some embodiments, the monocytes exhibit a lower expression level of M-CSFR, GM-CSFR, IL-6R, IL-10R, and/or IL-4R (e.g., at least about 10%, 20%, 30%, 40%, 50%, or 60% lower) at the time when they are obtained from the individual as compared to the monocytes obtained from a reference individual (e.g., a healthy individual). In some embodiments, the method further comprises administering a second therapy that induced immunogenic cell death (e.g., radiotherapy). In some embodiments, the method comprises administering the APCs and a radiotherapy concurrently, simultaneously, or subsequently. In some embodiments, the APCs have not been preloaded with a disease or condition associated antigen (e.g., tumor antigen or a virus antigen) prior to the administration. In some embodiments, the APCs have been preloaded with a disease or condition associated antigen (e.g., tumor antigen or a virus antigen) prior to the administration.

In some embodiments, there is provided a method of treating a disease or condition (e.g., a cancer, e.g., a virus infection) in a patient, comprising administering to the patient a population of antigen presenting cells (APCs), wherein the APCs are derived from monocytes obtained from an individual (e.g., a cancer patient or a virus infected patient), wherein the APCs are obtained by a) contacting a population of monocytes obtained from an individual with a plurality of survival, differentiation and/or maturation factors (“S/D/M factors”) separately or simultaneously, wherein the plurality of S/D/M factors comprise: 1) an IL-10 receptor (IL-10R) activator and 2) one or more agents selected from the group consisting of: an IL-4 receptor (IL-4R) activator, a TNFα receptor (TNFR) activator, and an interferon γ (IFNγ) receptor (IFNGR) activator, thereby obtaining a population of APCs. In some embodiments, the method further comprises administering a second therapy that induced immunogenic cell death (e.g., radiotherapy). In some embodiments, the method comprises administering the APCs and a radiotherapy concurrently, simultaneously, or subsequently. In some embodiments, the APCs have not been preloaded with a disease or condition associated antigen (e.g., tumor antigen or a virus antigen) prior to the administration. In some embodiments, the APCs have been preloaded with a disease or condition associated antigen (e.g., tumor antigen or a virus antigen) prior to the administration.

In some embodiments, there is provided a method of treating a disease or condition (e.g., a cancer, e.g., a virus infection) in a patient, comprising administering to the patient a population of antigen presenting cells (APCs), wherein the APCs are derived from monocytes obtained from an individual (e.g., a cancer patient or a virus infected patient), wherein the APCs a) express a high level of one or more antigen presentation molecule, wherein the antigen presentation molecule is selected from the group consisting of: MHCI, MHCII, CD86, CD80, OX40L, ICAML, ICOSL, and CD40, and/or b) a low level of an inhibitory signaling molecule, wherein the inhibitory signaling molecule is selected from the group consisting of: TGFβR, SIRPα, LILRB (LILRB1 and/or LILRB2) and Siglec 10, wherein the APCs have been preloaded with a disease or condition associated antigen (e.g., tumor antigen or a virus antigen) prior to the administration. In some embodiments, the monocytes exhibit a lower expression level of M-CSFR, GM-CSFR, IL-6R, IL-10R, and/or IL-4R (e.g., at least about 10%, 20%, 30%, 40%, 50%, or 60% lower) at the time when they are obtained from the individual as compared to the monocytes obtained from a reference individual (e.g., a healthy individual).

In some embodiments, there is provided a method of treating a disease or condition (e.g., a cancer, e.g., a virus infection) in a patient, comprising administering to the patient a population of activated immune cells, wherein the immune cells have been subject to a co-culture with a population of APCs, wherein the APCs are produced after contacting with an IL-10 receptor activator (IL-10R activator) and one or more of IFNγ receptor activator (IFNR activator), TNFα receptor activator (TNFR activator) and IL-4 receptor activator (IL-4R activator), and wherein the APCs have been pre-loaded with one or more peptides (e.g., tumor-associated peptides, e.g., neoantigen peptides, virus-specific peptides) associated with the disease or condition prior to the co-culture.

In some embodiments, there is provided a method of treating a disease or condition (e.g., a cancer, e.g., a virus infection) in a patient, comprising administering to the patient a population of activated immune cells (e.g., T cells), wherein the immune cells have been subject to a co-culture with a population of APCs, wherein the APCs are produced after contacting with IL-10 and one or more of IFNγ, TNFα and IL-4, and wherein the APCs have been pre-loaded with one or more peptides (e.g., tumor-associated peptides, e.g., neoantigen peptides, virus-specific peptides) associated with the disease or condition prior to the co-culture. In some embodiments, the immune cells are T cells. In some embodiments, the immune cells are T cells (e.g., CD3 T cells, e.g., CD4 T cells, e.g., CD8 T cells, e.g., both CD4 and CD8 T cells, e.g., TILs) obtained from the peripheral blood from the patient. In some embodiments, the immune cells are T cells (e.g., CD3 T cells, e.g., CD4 T cells, e.g., CD8 T cells, e.g., both CD4 and CD8 T cells, e.g., TILs) obtained from the peripheral blood from an individual different from the patient (optionally with a matching HLA type). In some embodiments, the APCs and the activated immune cells are derived from the same individual. In some embodiments, the APCs and the activated immune cells are derived from the different individuals (optionally with a matching HLA type). In some embodiments, the APCs are produced after contacting with IL-10, IFNγ, TNFα, and IL-4. In some embodiments, the APCs are produced after contacting with IL-10, IFNγ, TNFα, GM-CSF, IL-6 and IL-4. In some embodiments, the APCs are produced after contacting with one or more of the refinement factors described in Section II. In some embodiments, the activated immune cells are administered intratumorally, intraperitoneally, or intravenously. In some embodiments, the activated immune cells are administered at about 10⁷ to 10⁹ cells per dose. In some embodiments, the methods of treatment described herein further comprise treating the patient with chemotherapy, radiation therapy, or an immune checkpoint inhibitor. In some embodiments, the method comprises treating the patient with irradiation. In some embodiments, the site of irradiation is different from the site of the cancer to be treated.

In some embodiments, there is provided a method of treating a virus-related cancer in a patient, comprising administering to the patient a population of activated T cells, wherein the T cells have been subject to a co-culture with a population of APCs, wherein the APCs are produced after contacting with IL-10 and one or more of IFNγ, TNFα and IL-4, and wherein the APCs have been pre-loaded with one or more tumor-associated peptides (e.g., neoantigen peptides) associated with the virus-related cancer prior to the co-culture, wherein virus antigen reactive T cells have been removed from the activated T cell population prior to the administration. In some embodiments, the APCs are derived from the patient. In some embodiments, the activated T cells are derived from the patient. In some embodiments, the APCs and the activated T cells are both derived from the patient. In some embodiments, the activated immune cells are administered intratumorally, intraperitoneally, or intravenously. In some embodiments, the activated immune cells are administered at about 10⁷ to 10⁹ cells per dose. In some embodiments, the methods of treatment described herein further comprise treating the patient with chemotherapy, radiation therapy, or an immune checkpoint inhibitor. In some embodiments, the method comprises treating the patient with irradiation. In some embodiments, the site of irradiation is different from the site of the cancer to be treated.

In some embodiments, there is provided a method of treating a liver cancer associated with a virus (e.g., hepatitis B virus or hepatitis C virus) in a patient, comprising administering to the patient a population of activated T cells, wherein the T cells have been subject to a co-culture with a population of APCs, wherein the APCs are produced after contacting with IL-10 and one or more of IFNγ, TNFα and IL-4, and wherein the APCs have been pre-loaded with one or more tumor-associated peptides (e.g., neoantigen peptides) prior to the co-culture, wherein virus antigen reactive T cells have been removed from the activated T cell population prior to the administration. In some embodiments, the APCs are derived from the patient. In some embodiments, the activated T cells are derived from the patient. In some embodiments, the APCs and the activated T cells are both derived from the patient. In some embodiments, the activated immune cells are administered intratumorally, intraperitoneally, or intravenously. In some embodiments, the activated immune cells are administered at about 10⁷ to 10⁹ cells per dose. In some embodiments, the methods of treatment described herein further comprise treating the patient with chemotherapy, radiation therapy, or an immune checkpoint inhibitor. In some embodiments, the method comprises treating the patient with irradiation. In some embodiments, the site of irradiation is different from the site of the cancer to be treated.

### Patient

In some embodiments, the patient has a solid tumor. In some embodiments, the patient has a hematological cancer.

In some embodiments, the patient has an advanced cancer. In some embodiments, the patient has a late stage cancer. In some embodiments, the patient has a cancer that is in stage II, III or IV. In some embodiments, the patient has an inoperable tumor and/or metastases. In some embodiments, the patient is a terminally ill patient.

In some embodiments, the patient is a female. In some embodiments, the patient is a male.

In some embodiments, the patient is a human. In some embodiments, the patient is at least about 50, 55, 60, 65, 70 or 75 years old.

### Cancer

As discussed above, the methods of treatment that involve immune cells (such as T cells) activated by APCs produced by various methods described herein are applicable to all types of cancer.

In some embodiments, the cancer is a solid tumor. In some embodiments, the cancer is a hematologic cancer.

In some embodiments, the cancer is an advanced cancer. In some embodiments, the cancer is a late stage cancer. In some embodiments, the cancer is in stage II, III or IV. In some embodiments, the cancer is an inoperable tumor and/or is malignant.

In some embodiments, the cancer has been subjected to and/or failed one or more prior therapy (e.g., an immune checkpoint blockage therapy (e.g., an PD-1 antibody), a chemotherapy, a surgery, a cell therapy (e.g., an allogenic NK cell infusion therapy)).

In some embodiments, the cancer is a recurrent or refractory cancer.

Examples of cancers described herein include, but are not limited to, adrenocortical carcinoma, agnogenic myeloid metaplasia, AIDS-related cancers (e.g., AIDS-related lymphoma), anal cancer, appendix cancer, astrocytoma (e.g., cerebellar and cerebral), basal cell carcinoma, bile duct cancer (e.g., extrahepatic), bladder cancer, bone cancer, (osteosarcoma and malignant fibrous histiocytoma), brain tumor (e.g., glioma, brain stem glioma, cerebellar or cerebral astrocytoma (e.g., pilocytic astrocytoma, diffuse astrocytoma, anaplastic (malignant) astrocytoma), malignant glioma, ependymoma, oligodenglioma, meningioma, craniopharyngioma, hacmangioblastomas, medulloblastoma, supratentorial primitive neuroectodermal tumors, visual pathway and hypothalamic glioma, and glioblastoma), breast cancer, bronchial adenomas/carcinoids, carcinoid tumor (e.g., gastrointestinal carcinoid tumor), carcinoma of unknown primary, central nervous system lymphoma, cervical cancer, colon cancer, colorectal cancer, chronic myeloproliferative disorders, endometrial cancer (e.g., uterine cancer), ependymoma, esophageal cancer, Ewing's family of tumors, eye cancer (e.g., intraocular melanoma and retinoblastoma), gallbladder cancer, gastric (stomach) cancer, gastrointestinal carcinoid tumor, gastrointestinal stromal tumor (GIST), germ cell tumor, (e.g., extracranial, extragonadal, ovarian), gestational trophoblastic tumor, head and neck cancer, hepatocellular (liver) cancer (e.g., hepatic carcinoma and heptoma), hypopharyngeal cancer, islet cell carcinoma (endocrine pancreas), laryngeal cancer, laryngeal cancer, leukemia, lip and oral cavity cancer, oral cancer, liver cancer, lung cancer (e.g., small cell lung cancer, non-small cell lung cancer, adenocarcinoma of the lung, and squamous carcinoma of the lung), lymphoid neoplasm (e.g., lymphoma), medulloblastoma, melanoma, mesothelioma, metastatic squamous neck cancer, mouth cancer, multiple endocrine neoplasia syndrome, myelodysplastic syndromes, myelodysplastic/myeloproliferative diseases, nasal cavity and paranasal sinus cancer, nasopharyngeal cancer, neuroblastoma, neuroendocrine cancer, oropharyngeal cancer, ovarian cancer (e.g., ovarian epithelial cancer, ovarian germ cell tumor, ovarian low malignant potential tumor), pancreatic cancer, parathyroid cancer, penile cancer, cancer of the peritoneal, pharyngeal cancer, pheochromocytoma, pincoblastoma and supratentorial primitive neuroectodermal tumors, pituitary tumor, pleuropulmonary blastoma, lymphoma, primary central nervous system lymphoma (microglioma), pulmonary lymphangiomyomatosis, rectal cancer, renal cancer, renal pelvis and ureter cancer (transitional cell cancer), rhabdomyosarcoma, salivary gland cancer, skin cancer (e.g., non-melanoma (e.g., squamous cell carcinoma), melanoma, and Merkel cell carcinoma), small intestine cancer, squamous cell cancer, testicular cancer, throat cancer, thymoma and thymic carcinoma, thyroid cancer, tuberous sclerosis, urethral cancer, vaginal cancer, vulvar cancer, Wilms' tumor, and post-transplant lymphoproliferative disorder (PTLD), abnormal vascular proliferation associated with phakomatoses, edema (such as that associated with brain tumors), and Meigs' syndrome.

In some embodiments, the cancer is a virus-infection-related cancer. In some embodiments, the cancer is a human papillomavirus (HPV)-related cancer (e.g., HPV-related cervical cancer, e.g., HPV-related head and neck cancer, e.g., HPV related squamous cell carcinoma). In some embodiments, the cancer is human herpes virus 8 (HHV8) related cancer (e.g., Kaposi sarcoma). In some embodiments, the cancer is human T-lymphotrophic virus (HTLV-1)-related cancer (e.g., adult T cell leukemia or lymphoma). In some embodiments, the cancer is Epstein-Barr virus (EBV) related cancer (e.g., Burkitt lymphoma, Hodgkin's and non-Hodgkin's lymphoma, stomach cancer). In some embodiments, the cancer is hepatitis B virus (HBV) related cancer (e.g., liver cancer). In some embodiments, the cancer is hepatitis C virus) related cancer (e.g., liver cancer, non-Hodgkin's lymphoma).

### Dosing and Method of Administering the Activated Immune Cells (e.g., T Cells)

The activated immune cells can be administered at any desired dosage, which in some aspects includes a desired dose or number of cells or cell type(s). Thus, the dosage of cells in some embodiments is based on a total number of cells (or number per kg body weight) and/or a desired ratio of the individual populations. In some embodiments, the dosage of cells is based on a desired total number (or number per kg of body weight) of cells in the individual populations or of individual cell types.

In certain embodiments, the activated immune cells (e.g., T cells, e.g., CD4 and/or CD8 T cells, e.g., TILs), are administered to the subject at a range of about one million to about 100 billion cells and/or that amount of cells per kilogram of body weight, such as, e.g., 1 million to about 50 billion cells (e.g., about 5 million cells, about 25 million cells, about 500 million cells, about 1 billion cells, about 5 billion cells, about 20 billion cells, about 30 billion cells, about 40 billion cells, or a range defined by any two of the foregoing values), such as about 10 million to about 100 billion cells (e.g., about 20 million cells, about 30 million cells, about 40 million cells, about 60 million cells, about 70 million cells, about 80 million cells, about 90 million cells, about 10 billion cells, about 25 billion cells, about 50 billion cells, about 75 billion cells, about 90 billion cells, or a range defined by any two of the foregoing values), and in some cases about 100 million cells to about 50 billion cells (e.g., about 120 million cells, about 250 million cells, about 350 million cells, about 450 million cells, about 650 million cells, about 800 million cells, about 900 million cells, about 3 billion cells, about 30 billion cells, about 45 billion cells) or any value in between these ranges and/or per kilogram of body weight. Dosages may vary depending on attributes particular to the disease or disorder and/or patient and/or other treatments.

In some embodiments, for example, where the subject is a human, the dose includes fewer than about 1×10⁹ total activated immune cells, e.g., in the range of about 1×10⁶ to 5×10⁸ such cells, such as 2×10⁶, 5×10⁶, 1×10⁷, 5×10⁷, 1×10⁸, 5×10⁸ or 1×10⁹ total such cells, or the range between any two of the foregoing values.

In some embodiments, the activated immune cells are administered at about 10⁷ to 10⁹ cells per dose.

In some embodiments, the method of treatment comprises administration of a dose comprising a number of cells from about 1×10⁶ to 1×10⁹ (e.g., 10⁶ to 10⁷, 10⁷ to 10⁸, or 10⁸ to 10⁹) total activated immune cells (e.g., total CD3 T cells, both CD4 and CD8 T cells, CD4 T cells only, CD8 T cells only, or TILs).

In some embodiments, the dose of the activated immune cells is administered to the subject as a single dose or is administered only one time within a period of two weeks, one month, three months, six months, 1 year or more.

In some embodiments, the dose of total activated immune cells is within a range of between at or about 10⁴ and at or about 10⁹ cells/kilograms (kg) body weight, such as between 10⁵ and 10⁶ cells/kg body weight, for example, at or about 1×10⁵ cells/kg, 1.5×10⁵ cells/kg. 2×10⁵ cells/kg, or 1×10⁶ cells/kg body weight. For example, in some embodiments, the activated immune cells are administered at, or within a certain range of error of, between at or about 10⁴ and at or about 10⁹ T cells/kilograms (kg) body weight, such as between 10⁵ and 10⁶ T cells/kg body weight, for example, at or about 1×10⁵ T cells/kg, 1.5×10⁵ T cells/kg, 2×10⁵ T cells/kg, or 1×10⁶ T cells/kg body weight.

In some embodiments, the activated immune cells are administered at or within a certain range of error of between at or about 10⁴ and at or about 10⁹ CD4+ and/or CD8+ cells/kilograms (kg) body weight, such as between 10⁵ and 10⁶ CD4+ and/or CD8+ cells/kg body weight, for example, at or about 1×10⁵ CD4+ and/or CD8+ cells/kg. 1.5×10⁵ CD4+ and/or CD8+ cells/kg, 2×10⁵ CD4+ and/or CD8+ cells/kg, or 1×10⁶ CD4+ and/or CD8+ cells/kg body weight.

In some embodiments, the activated immune cells are administered at or within a certain range of error of, greater than, and/or at least about 1×10⁶, about 2.5×10⁶, about 5×10⁶, about 7.5×10⁶, or about 9×10⁶ CD4+ cells, and/or at least about 1×10⁶, about 2.5×10⁶, about 5×10⁶, about 7.5×10⁶, or about 9×10⁶ CD8+ cells, and/or at least about 1×10⁶, about 2.5×10⁶, about 5×10⁶, about 7.5×10⁶, or about 9×10⁶ T cells. In some embodiments, the activated immune cells are administered at or within a certain range of error of between about 10⁸ and 10¹² or between about 10¹⁰ and 10¹¹ T cells, between about 10⁸ and 10¹² or between about 10¹⁰ and 10¹¹ CD4+ cells, and/or between about 10⁸ and 10¹² or between about 10¹⁰ and 10¹¹ CD8+ cells.

For the prevention or treatment of disease, the appropriate dosage may depend on the type of disease to be treated, the type of cells or recombinant receptors, the severity and course of the disease, whether the activated immune cells are administered for preventive or therapeutic purposes, previous therapy, the subject's clinical history and response to the activated immune cells, and the discretion of the attending physician. The compositions and cells are in some embodiments suitably administered to the subject at one time or over a series of treatments.

In some embodiments, the APCs described herein are administered to the subject at a range of about 5000 to about 10,000 cells/mm³ per tumor mass.

In some aspects, the size of the dose is determined based on one or more criteria such as response of the subject to prior treatment, e.g., chemotherapy, disease burden in the subject, such as tumor load, bulk, size, or degree, extent, or type of metastasis, stage, and/or likelihood or incidence of the subject developing toxic outcomes, e.g., CRS, macrophage activation syndrome, tumor lysis syndrome, neurotoxicity, and/or a host immune response against the activated immune cells being administered.

In some aspects, the size of the dose is determined by the burden of the disease or condition in the subject. For example, in some aspects, the number of cells administered in the dose is determined based on the tumor burden that is present in the subject immediately prior to administration of the initiation of the dose of cells. In some embodiments, the size of the first and/or subsequent dose is inversely correlated with disease burden. In some aspects, as in the context of a large disease burden, the subject is administered a low number of cells. In other embodiments, as in the context of a lower disease burden, the subject is administered a larger number of cells.

The activated immune cells can be administered by any suitable means, for example, by bolus infusion, by injection, e.g., intravenous or subcutaneous injections, intraocular injection, periocular injection, subretinal injection, intravitreal injection, trans-septal injection, subscleral injection, intrachoroidal injection, intracameral injection, subconjectval injection, subconjuntival injection, sub-Tenon's injection, retrobulbar injection, peribulbar injection, or posterior juxtascleral delivery. In some embodiments, they are administered by parenteral, intrapulmonary, and intranasal, and, if desired for local treatment, intralesional administration. Parenteral infusions include intramuscular, intravenous, intraarterial, intraperitoneal, or subcutaneous administration. In some embodiments, a given dose is administered by a single bolus administration of the activated immune cells. In some embodiments, it is administered by multiple bolus administrations of the activated immune cells, for example, over a period of no more than 3 days, or by continuous infusion administration of the activated immune cells.

In some embodiments, the activated immune cells are administered intratumorally, intraperitoneally, or intravenously.

### Combination Therapy

In some embodiments, the APCs (e.g., antigen-challenged or naïve) or activated immune cells are administered as part of a combination treatment, such as simultaneously with, concurrently with, or sequentially with, another therapeutic intervention (i.e., a second therapy), such as an antibody or engineered cell or receptor or agent, such as a cytotoxic or therapeutic agent. In some embodiments, the APCs or activated immune cells are administered prior to another therapeutic intervention. In some embodiments, the APCs or activated immune cells are administered after another therapeutic intervention. The APCs or activated immune cells in some embodiments are co-administered with one or more additional therapeutic agents or in connection with another therapeutic intervention, simultaneously, concurrently, or sequentially in any order. In some contexts, the APCs or activated immune cells are co-administered with another therapy sufficiently close in time such that the cell populations enhance the effect of one or more additional therapeutic agents, or vice versa. In some embodiments, the APCs or activated immune cells are administered prior to the one or more additional therapeutic agents. In some embodiments, the APCs or activated immune cells are administered after the one or more additional therapeutic agents. In some embodiments, the one or more additional agents to be administered includes a cytokine, such as IL-2, for example, to enhance persistence of the activated immune cells. In some embodiments, the methods comprise administration of a chemotherapeutic agent.

In some embodiments, the second therapy comprises a chemotherapy, radiation therapy, or an immune checkpoint inhibitor. In some embodiments, the second therapy is gene therapy (e.g., mRNA-based gene therapy). In some embodiments, the second therapy comprises administration of a cancer vaccine (such as mRNA-based cancer vaccine or DNA-based cancer vaccine). In some embodiments, the second therapy comprises administration of an oncolytic virus. In some embodiments, the APCs or immune cells are administered prior to the administration of the second therapy. In some embodiments, the APCs or immune cells are administered in a neoadjuvant setting.

In some embodiments, the second therapy comprises treating the patient with irradiation. In some embodiments, the site of irradiation is different from the site of the cancer to be treated.

Thus, for example, in some embodiments, there is provided a method of treating an individual having cancer, comprising administering to the individual an effective amount of an APCs or immune cell activated by any of the methods described herein, wherein the individual is treated with a radiation therapy, and wherein the site of the irradiation is different from the site of the cancer to be treated.

In some embodiments, the radiation therapy is selected from the group consisting of external-beam radiation therapy, internal radiation therapy (brachytherapy), intraoperative radiation therapy (IORT), systemic radiation therapy, radioimmunotherapy, and administration of radiosensitizers and radioprotectors. In some embodiments, the radiation therapy is external-beam radiation therapy, optionally comprising three-dimensional conformal radiation therapy (3D-RT), intensity modulated radiation therapy (IMRT), photon beam therapy, image-guided radiation therapy (IGRT), and sterotactic radiation therapy (SRT). In some embodiments, the radiation therapy is brachytherapy, optionally comprising interstitial brachytherapy, intracavitary brachytherapy, intraluminal radiation therapy, and radioactively tagged molecules given intravenously.

### VI. Compositions Comprising a Plurality of Survival, Differentiation and/or Maturation Factors (“S/D/M Factors”)

The present application also provides compositions (e.g., cell culture medium) comprising a plurality of survival, differentiation and/or maturation factors (“S/D/M factors”) wherein the plurality of S/D/M factors comprise as described above.

In some embodiments, there is provided a composition (e.g., a cell culture medium) comprising a plurality of survival, differentiation and/or maturation factors (“S/D/M factors”): 1) an IL-10 receptor (IL-10R) activator and 2) one or more agents selected from the group consisting of: an IL-4 receptor (IL-4R) activator, a TNFα receptor (TNFR) activator, and an interferon γ (IFNγ) receptor (IFNGR) activator.

In some embodiments, the IL-10R activator is selected from the group consisting of: an IL-10 (e.g., a pegylated IL-10, e.g., pegilodecakin or AM0010), an IL-10 family member (e.g., IL-19, IL-20, IL-22, IL-24, IL-26, IL-28), an IL-10R agonist antibody, a small molecule activator of IL-10R, and an activator of the IL-10R downstream STAT3 (e.g., Long noncoding RNA (LncRNA) PVT1, NEAT1, FEZF1-AS1, UICC). In some embodiments, the IL-10R activator is IL-10. In some embodiments, the IL-10 is a human IL-10 or a human recombinant IL-10. In some embodiments, the IL-10 is present in the medium at a concentration of at least about 2 ng/ml, optionally at least about 10 ng/ml, further optionally about 10 ng/ml to about 200 ng/ml (e.g., about 20 ng/ml).

In some embodiments, the IFNGR activator is selected from the group consisting of IFNγ, an IFNGR agonist antibody, and a small molecule activator of IFNGR. In some embodiments, the IFNGR activator is IFNγ. In some embodiments, the IFNγ is a human IFNγ or a human recombinant IFNγ. In some embodiments, the IFNγ is present in the medium at a concentration of at least about 5 ng/ml, optionally at least about 10 ng/ml, further optionally about 10 ng/ml to about 200 ng/ml (e.g., about 50-100 ng/ml).

In some embodiments, the IL-4R activator is selected from the group consisting of IL-4, IL-13, an IL-4R agonist antibody, and a small molecule activator of IL-4R. In some embodiments, the IL-4R activator is IL-4. In some embodiments, the IL-4 is a human IL-4 or a human recombinant IL-4. In some embodiments, the IL-4 is present in the medium at a concentration of at least about 15 μg/ml, optionally at least about 30 μg/ml, further optionally about 30 μg/ml to about 1 ng/ml (e.g., about 100 μg/ml to about 1 ng/ml).

In some embodiments, the TNFR activator is selected from the group consisting of TNFα, a TNFR agonist antibody, and a small molecule activator of TNFR. In some embodiments, the TNFR activator is TNFα. In some embodiments, the TNFα is a human TNFα or a human recombinant TNFα. In some embodiments, the TNFα is present in the medium at a concentration of at least about 0.5 ng/ml, optionally at least about 1 ng/ml, further optionally about 0.5 ng/ml to about 30 ng/ml (e.g., about 1-10 ng/ml).

In some embodiments, the plurality of S/D/M factors comprise two or more agents selected from the group consisting of an IL-4R activator, a TNFR activator, and an IFNGR activator. In some embodiments, the plurality of S/D/M factors comprise a TNFR activator, and an IFNGR activator.

In some embodiments, the plurality of S/D/M factors comprises IL-10, IL-4, TNFα, and IFNγ.

In some embodiments, the plurality of the S/D/M factors further comprise a GM-CSF receptor (GM-CSFR) activator.

In some embodiments, the GM-CSFR activator is selected from the group consisting of GM-CSF, a GM-CSFR agonist antibody, and a small molecule activator of GM-CSFR. In some embodiments, the GM-CSFR activator is GM-CSF. In some embodiments, the GM-CSF is a human GM-CSF or a human recombinant GM-CSF. In some embodiments, the GM-CSF is present in the medium at a concentration of at least about 30 μg/ml, optionally at least about 50 μg/ml, further optionally about 100 μg/ml to about 1 ng/ml (e.g., about 100 μg/ml to about 500 μg/ml, e.g., about 300 μg/ml).

In some embodiments, the plurality of the S/D/M factors further comprise an IL-6 receptor (IL-6R) activator, optionally wherein the IL-6R activator is selected from the group consisting of IL-6, an IL-6R agonist antibody, and a small molecule activator of IL-6R. In some embodiments, the IL-6R activator is IL-6. In some embodiments, the IL-6 is a human IL-6 or a human recombinant IL-6. In some embodiments, the IL-6 is present in the medium at a concentration of at least about 1 μg/ml, optionally at least about 5 μg/ml, further optionally about 5 μg/ml to about 100 μg/ml (e.g., about 10-50 pg/ml, e.g., about 30 μg/ml).

In some embodiments, the plurality of S/D/M factors comprises IL-10, IL-4, TNFα, IL-6, GM-CSF, and IFNγ.

In some embodiments, the plurality of maturation factors further comprises one or more of: IL-2, IL-4, IL-17, and M-CSF, agonist antibodies thereof, or small molecule activators thereof.

In some embodiments, there is provided a composition (e.g., a cell culture medium) comprising an IL-10 receptor (IL-10R) activator, optionally the IL-10R activator is IL-10 (e.g., a human IL-10, or a human recombinant IL-10), further optionally the IL-10 is present in the medium at a concentration of at least about 2 ng/ml (e.g., at least about 10 ng/ml, e.g., at least about 20 ng/ml, e.g., about 10 ng/ml to about 200 ng/ml, e.g., about 20 ng/ml), optionally wherein the medium is particularly for cancer cells (e.g., monocytes obtained from cancer patients) or cells (e.g., monocytes) that express a low level of IL-10R (e.g., at least 20%, 30%, 40%, 50% lower than that of corresponding cells from a reference individual (e.g., a healthy individual).

The composition described herein can be prepared by combining each of the components into a single composition. In some embodiments, the composition is prepared by culturing an immune cell (e.g., a T cell, e.g., a CD4 T cell, e.g., a CD8 T cell) and obtaining the cell culture supernatant. In some embodiments, the supernatant can be further supplemented with an additional component (or an additional amount of a component already exist in the supernatant) or modified to remove a component to obtain the desired composition.

In some embodiments, there is provided a composition (e.g., a cell culture medium) derived from a culture (e.g., supernatant) of T cells after being treated with anti-CD3 and anti-CD28 antibodies, wherein the medium comprises IL-10. In some embodiments, the T cells are CD4 T cells. In some embodiments, the T cells are CD8 T cells. In some embodiments, the T cells are isolated from PBMC of the same individual or a different individual and have not been previously treated with anti-CD3 and/or anti-CD28 antibodies prior to the treatment. In some embodiments, the T cells are isolated from PBMC of the same individual or a different individual and have been previously treated with anti-CD3 and/or anti-CD28 antibodies prior to the treatment. In some embodiments, the medium is derived from the culture after the T cells are treated with anti-CD3 and anti-CD28 antibodies for about 1-3 days, optionally for about 2 days. In some embodiments, at least one or more molecules (e.g., IL-2) is removed from the culture of the T cells. In some embodiments, the one or more molecules are selected from the group consisting of IL-2, M-CSF, IL-12, and IL-17 (e.g., IL-17A).

### EXEMPLARY EMBODIMENTS

Embodiment 1. A method of stimulating a population of monocytes from an individual to produce a population of antigen presenting cells (“APCs”), comprising contacting the population of monocytes with a plurality of survival, differentiation and/or maturation factors (“S/D/M factors”) separately or simultaneously, wherein the plurality of S/D/M factors comprise: 1) an IL-10 receptor (IL-10R) activator and 2) one or more agents selected from the group consisting of: an IL-4 receptor (IL-4R) activator, a TNFα receptor (TNFR) activator, and an interferon γ (IFNγ) receptor (IFNGR) activator, thereby obtaining a population of APCs.

Embodiment 2. The method of embodiment 1, wherein the IL-10R activator is selected from the group consisting of: an IL-10, an IL-10 family member, an IL-10R agonist antibody, a small molecule activator of IL-10R, and an activator of the IL-10R downstream STAT3, optionally wherein the activator of the IL-10R downstream STAT3 is selected from an IL-10 family cytokine, an IL-12 family cytokine, an IL-6 family cytokine, a small molecule STAT3 activator, and G-CSF.

Embodiment 3. The method of embodiment 1, wherein the IL-10R activator is selected from the group consisting of IL-10, IL-22, IL-19, IL20, IL-24, IL12, IL-23, IL-6, colivelin TFA, Garcinone D, and G-CSF, optionally wherein the IL-10R activator is IL-10, IL-22, IL-19, IL-20, IL-24, IL-12, IL-23, Colivelin TFA, or Garcinone D.

Embodiment 4. The method of any one of embodiments 1-3, wherein the plurality of S/D/M factors comprise an IL-4R activator, optionally wherein the IL-4R activator is selected from the group consisting of IL-4, an IL-4R agonist antibody, and a small molecule activator of IL-4R.

Embodiment 5. The method of embodiment 4, wherein the IL-4R activator is IL-4.

Embodiment 6. The method of any one of embodiments 1-5, wherein the plurality of S/D/M factors comprise a TNFR activator, optionally wherein the TNFR activator is selected from the group consisting of TNFα, a TNFR agonist antibody, and a small molecule activator of TNFR.

Embodiment 7. The method of embodiment 6, wherein the TNFR activator is TNFα.

Embodiment 8. The method of any one of embodiments 1-7, wherein the plurality of S/D/M factors comprise an IFNGR activator, optionally wherein the IFNGR activator is selected from the group consisting of IFNγ, an IFNGR agonist antibody, and a small molecule activator of IFNGR.

Embodiment 9. The method of embodiment 8, wherein the IFNGR activator is IFNγ.

Embodiment 10. The method of any one of embodiments 1-9, wherein the plurality of S/D/M factors are present in a single composition.

Embodiment 11. The method of any one of embodiment 1-10, wherein at least one of the plurality of S/D/M factors is provided separately from one of other S/D/M factors in the plurality of S/D/M factors.

Embodiment 12. The method of any one of embodiments 1-11, wherein the plurality of S/D/M factors comprise two or more agents selected from the group consisting of an IL-4R activator, a TNFR activator, and an IFNGR activator.

Embodiment 13. The method of embodiment 12, wherein the plurality of S/D/M factors comprise an IL-10R activator, TNFα, and IFNγ, optionally wherein the plurality of S/D/M factors comprise an IL-10 family cytokine (e.g., IL-10, IL-22, IL-19, IL-24, IL-20, IL-26), TNFα, and IFNγ, optionally wherein the plurality of S/D/M factors comprise an IL-10R activator, IL-4, TNFα, and IFNγ.

Embodiment 14. The method of any one of embodiments 1-13, wherein the plurality of the S/D/M factors further comprise a GM-CSF receptor (GM-CSFR) activator.

Embodiment 15. The method of embodiment 14, wherein the GM-CSFR activator is selected from the group consisting of GM-CSF, a GM-CSFR agonist antibody, and a small molecule activator of GM-CSFR.

Embodiment 16. The method of embodiment 15, wherein the GM-CSFR activator is GM-CSF.

Embodiment 17. The method of any one of embodiments 1-16, wherein the plurality of the S/D/M factors further comprise an IL-6 receptor (IL-6R) activator, optionally wherein the IL-6R activator is selected from the group consisting of IL-6, an IL-6R agonist antibody, and a small molecule activator of IL-6R.

Embodiment 18. The method of embodiment 17, wherein the IL-6R activator is IL-6.

Embodiment 19. The method of any one of embodiments 1-18, wherein the plurality of S/D/M factors are derived from a culture of T cells after being treated with anti-CD3 and anti-CD28 antibodies, optionally the plurality of S/D/M factors are derived from the supernatant of the culture.

Embodiment 20. The method of embodiment 19, wherein the T cells are isolated from PBMC of the same individual or a different individual, and optionally wherein the T cells have not been previously treated with anti-CD3 and/or anti-CD28 antibodies prior to the treatment.

Embodiment 21. The method of embodiment 19 or embodiment 20, wherein the plurality of S/D/M factors are derived from the culture after the T cells are treated with anti-CD3 and anti-CD28 antibodies for about 1-3 days, optionally for about 2 days.

Embodiment 22. The method of any one of embodiments 1-21, wherein the monocytes are cultured for about 2-3 days in the presence of the S/D/M factors or the medium derived from the culture of T cells.

Embodiment 23. The method of any one of embodiments 1-22, further comprising contacting the population of monocytes with a plurality of refinement factors selected from the group consisting of type-I interferon, IFNγ, TNFα, a TLR ligand, CD40L or a CD40-ligating antibody, an anti-PD-L1 antibody, and TPI-1, optionally wherein the type-I interferon comprises IFNα and/or IFNβ, and optionally wherein the TLR ligand is poly IC, CpG, or LPS.

Embodiment 24. The method of embodiment 23, wherein the plurality of refinement factors are provided after the plurality of monocytes are contacted with the plurality of S/D/M factors or the medium derived from the culture of T cells, thereby producing the population of APCs, and wherein the population of APCs are cultured for about 1-5 days in the presence of the plurality of the refinement factors, optionally wherein the population of the APCs are cultured for about one day.

Embodiment 25. The method of embodiment 23 or embodiment 24, wherein the plurality of refinement factors are provided when a) at least about 50% of the monocytes survive, b) at least about 30% of the population of APCs exhibit a dendritic cell morphology and/or c) the population of APCs express i) a high level of one or more molecules selected from the group consisting of MHC I, MHC II, CD80, CD86, and/or CD40, and/or ii) a low level of SIRPα.

Embodiment 26. The method of any one of embodiments 23-25, wherein the refinement factors comprise IFNα, IFNγ, and TNFα.

Embodiment 27. The method of embodiment 26, wherein the refinement factors further comprise at least two agents selected from the group consisting of poly IC, CpG. CD40L, R848, and an anti-PD-L1 antibody, optionally wherein the refinement factors comprise a SHP-1 inhibitor (e.g., TPI-1).

Embodiment 28. A method of promoting the survival of a population of monocytes from an individual in an in vitro culture, comprising cultivating the population of monocytes in a medium having one or more molecules that promote IL-10 receptor (IL-10R) expression on the monocytes.

Embodiment 29. The method of embodiment 28, wherein the one or more molecules comprises an IL-10R activator, optionally wherein the IL-10R activator is selected from the group consisting of: an IL-10, an IL-10 family member, an IL-10R agonist antibody, a small molecule activator of IL-10R, and an activator of the IL-10R downstream STAT3, further optionally the IL-10R activator is IL-10.

Embodiment 30. A method of promoting the survival of a population of monocytes from an individual in an in vitro culture, comprising cultivating the population of monocytes in a medium having an IL-10R activator, optionally wherein the IL-10R activator is selected from the group consisting of: an IL-10, an IL-10 family member, an IL-10R agonist antibody, a small molecule activator of IL-10R, and an activator of the IL-10R downstream STAT3, further optionally the IL-10R activator is IL-10.

Embodiment 31. The method of any one of embodiments 28-30, wherein the population of monocytes express a low level of IL-10R prior to contacting with the molecule.

Embodiment 32. The method of any one of embodiments 28-31, wherein the culture comprise a TNFα receptor (TNFR) activator, and/or an interferon γ (IFNγ) receptor (IFNGR) activator, optionally wherein the TNFR activator is selected from the group consisting of TNFα, a TNFR agonist antibody, and a small molecule activator of TNFR, and optionally wherein the IFNGR activator is selected from the group consisting of IFNγ, an IFNGR agonist antibody, and a small molecule activator of IFNGR, and further optionally the culture comprises TNFα and/or IFNγ.

Embodiment 33. A method of increasing expression of IL-10 receptor (IL-10R) in a population of monocytes from an individual having cancer, comprising contacting the population of monocytes with one or more agents selected from the group consisting of: an IL-10R activator, a TNFR activator, and an IFNGR activator.

Embodiment 34. A method of promoting the survival of a population of monocytes from an individual in an in vitro culture, comprising cultivating the population of monocytes in a medium comprising IL-10, TNFα, and IFNγ.

Embodiment 35. A method of promoting the differentiation of a population of monocytes from an individual to antigen presenting cells (“APCs”) in an in vitro culture, comprising cultivating the population of monocytes in a medium having one or more molecules selected from the group consisting of an IL-4 receptor (IL-4R) activator, a TNFα receptor (TNFR) activator, and an interferon γ (IFNγ) receptor (IFNGR) activator.

Embodiment 36. The method of embodiment 35, wherein the culture further comprises an IL-6 receptor (IL-6R) activator and/or a GM-CSF receptor (GM-CSFR) activator.

Embodiment 37. The method of any one of embodiments 1-36, wherein the plurality of monocytes are obtained from the peripheral blood of the individual, optionally wherein the monocytes express CD14, wherein they are obtained from the peripheral blood.

Embodiment 38. The method of any one of embodiments 1-37, wherein the individual has a cancer.

Embodiment 39. The method of embodiment 38, wherein the individual has a late stage cancer.

Embodiment 40. The method of any one of embodiments 1-39, wherein the individual has a solid tumor.

Embodiment 41. The method of any one of embodiments 1-40, wherein the individual has inoperable tumor and/or metastases.

Embodiment 42. The method of any one of embodiments 1-41, wherein the individual is a human.

Embodiment 43. A population of APCs produced by the method of any one of embodiments 1-27 and 35-42.

Embodiment 44. A population of APCs, wherein the APCs a) are MHC-I+/high, MHC-II+/high, and CD40+/high, b) are TLR2+/high and/or STING+/high, and c) LOX1+/high and/or uPAR+/high, and optionally wherein the expression level of CD40 on the APCs are at least 5-fold, 10-fold, 20-fold, 50-fold, or 100-fold higher than that on monocytes, M1 macrophages, M2 macrophages, and/or MoDCs.

Embodiment 45. A population of APCs, wherein the APCs express a higher level of one or more antigen presentation molecule, wherein the antigen presentation molecule is selected from the group consisting of: MHCI, MHCII, CD86, CD80, OX40L, ICAML, ICOSL, and CD40 than dendritic cells obtained from a healthy human and cultured with GM-CSF and IL-4 for about 5 days, optionally wherein the APCs are produced from monocytes in an ex vivo cell culture, further optionally wherein the monocytes are obtained from a cancer patient, optionally wherein the APCs express a low level of an inhibitory signaling molecule, wherein the inhibitory signaling molecule is selected from the group consisting of: TGFβR, SIRPα, LIIRBs and Siglec 10.

Embodiment 46. A method of activating a population of immune cells, comprising co-culturing the population of immune cells with the population of the APCs of any one of embodiments 43-45, wherein the APCs are pre-loaded with one or more neoantigen peptides.

Embodiment 47. The method of embodiment 46, wherein the method comprises contacting the APCs with a composition comprising a plurality of neoantigen peptides, and/or the APCs have been pre-incubated with the composition.

Embodiment 48. The method of embodiment 47, wherein the composition comprising a plurality of neoantigen peptides is a surgical resection of tumor tissue or a biopsy extract thereof.

Embodiment 49. The method of embodiment 47, wherein the composition comprising a plurality of neoantigen peptides is a mixture of tumor cells or extract thereof isolated from tumor tissue or biopsy.

Embodiment 50. The method of embodiment 49, wherein the composition comprising a plurality of neoantigen peptides is a mixture of isolated neoantigen peptides.

Embodiment 51. The method of embodiment 50, wherein the isolated neoantigen peptides are synthetic peptides.

Embodiment 52. The method of any one of embodiments 47-51, wherein the APCs are allowed to be in contact with the composition comprising a plurality of neoantigen peptides for about 4 to about 24 hours.

Embodiment 53. The method of any one of embodiments 46-52, wherein the immune cells are selected from the group consisting of PBMC, tumor infiltrating T cells (TIL), and T cells, optionally wherein the immune cells are T cells, optionally wherein the T cells are CD8 T cells and/or CD4 T cells, optionally the activating is performed for at least three rounds, wherein each round of co-culture takes at least about 5 days, wherein the co-culture for at least two of the three rounds do not comprises an anti-CD3 antibody or an anti-CD28 antibody.

Embodiment 54. The method of any one of embodiments 46-53, wherein the co-culturing was carried out for at least 24 hours.

Embodiment 55. The method of any one of embodiments 46-54, further comprising expanding the population of immune cells following the co-culturing step.

Embodiment 56. The method of embodiment 55, wherein expanding the population of immune cells comprises contacting the immune cells with a cytokine selected from the group consisting of IL-2, IL-7, and IL-15, optionally for about 2 to about 10 days.

Embodiment 57. The method of any one of embodiments 46-56, wherein the population of immune cells and the antigen presenting cells are derived from the same individual.

Embodiment 58. The method of any one of embodiments 46-57, wherein the population of immune cells and the antigen presenting cells are not derived from the same individual.

Embodiment 59. A population of activated immune cells obtained by the method of any one of embodiments 46-58.

Embodiment 60. A method of treating cancer in a patient, comprising administering to the patient a population of APCs of embodiment 43-45 and/or activated immune cells of embodiment 59.

Embodiment 61. The method of embodiment 60, wherein the APCs or activated immune cells are administered intratumorally, intraperitoneally, or intravenously.

Embodiment 62. The method of embodiment 61, wherein the activated immune cells are administered at about 10⁷ to 10⁹ cells per dose.

Embodiment 63. The method of any one of embodiments 60-62, further comprising treating the patient with chemotherapy, radiation therapy, or an immune checkpoint inhibitor.

Embodiment 64. The method of embodiment 63, wherein the method comprises treating the patient with irradiation.

Embodiment 65. The method of embodiment 64, wherein the site of irradiation is different from the site of the cancer to be treated.

Embodiment 66. The method of any one of embodiments 60-65, wherein the APCs or activated immune cells administered to the patient are derived from the patient.

Embodiment 67. The method of any one of embodiments 60-65, wherein the APCs or activated immune cells administered to the patient are not derived from the patient.

Embodiment 68. The method of any one of embodiments 60-67, wherein the cancer to be treated is a solid tumor.

Embodiment 69. A composition comprising a plurality of survival, differentiation and/or maturation factors (“S/D/M factors”), wherein the plurality of S/D/M factors comprise: 1) an IL-10 receptor (IL-10R) activator and 2) one or more agents selected from the group consisting of: an IL-4 receptor (IL-4R) activator, a TNFα receptor (TNFR) activator, and an interferon γ (IFNγ) receptor (IFNGR) activator.

Embodiment 70. The composition of embodiment 69, wherein the IL-10R activator is selected from the group consisting of: an IL-10, an IL-10 family member, an IL-10R agonist antibody, a small molecule activator of IL-10R, and an activator of the IL-10R downstream STAT3.

Embodiment 71. The composition of embodiment 70, wherein the IL-10R activator is IL-10, IL-22, IL-19, IL-20, IL-24, IL-12, IL-23, Colivelin TFA, or Garcinone D, optionally wherein the IL-10R activator is IL-10.

Embodiment 72. The composition of any one of embodiments 69-71, wherein the plurality of S/D/M factors comprise an IL-4R activator, optionally wherein the IL-4R activator is selected from the group consisting of IL-4, an IL-4R agonist antibody, and a small molecule activator of IL-4R.

Embodiment 73. The composition of embodiment 72, wherein the IL-4R activator is IL-4.

Embodiment 74. The composition of any one of embodiments 69-73, wherein the plurality of S/D/M factors comprise a TNFR activator, optionally wherein the TNFR activator is selected from the group consisting of TNFα, a TNFR agonist antibody, and a small molecule activator of TNFR.

Embodiment 75. The composition of embodiment 74, wherein the TNFR activator is TNFα.

Embodiment 76. The composition of any one of embodiments 69-75, wherein the plurality of S/D/M factors comprise an IFNGR activator, optionally wherein the IFNGR activator is selected from the group consisting of IFNγ, an IFNGR agonist antibody, and a small molecule activator of IFNGR.

Embodiment 77. The composition of embodiment 76, wherein the IFNGR activator is IFNγ.

Embodiment 78. The composition of any one of embodiments 69-77, wherein the plurality of S/D/M factors comprise two or more agents selected from the group consisting of an IL-4R activator, a TNFR activator, and an IFNGR activator.

Embodiment 79. The composition of embodiment 78, wherein the plurality of S/D/M factors comprises IL-10, IL-4, TNFα, and IFNγ.

Embodiment 80. The composition of embodiments 69-79, wherein the plurality of the S/D/M factors further comprise a GM-CSF receptor (GM-CSFR) activator.

Embodiment 81. The composition of embodiment 80, wherein the GM-CSFR activator is selected from the group consisting of GM-CSF, a GM-CSFR agonist antibody, and a small molecule activator of GM-CSFR.

Embodiment 82. The composition of embodiment 81, wherein the GM-CSFR activator is GM-CSF.

Embodiment 83. The composition of any one of embodiments 69-82, wherein the plurality of the S/D/M factors further comprise an IL-6 receptor (IL-6R) activator, optionally wherein the IL-6R activator is selected from the group consisting of IL-6, an IL-6R agonist antibody, and a small molecule activator of IL-6R.

Embodiment 84. The composition of embodiment 83, wherein the IL-6R activator is IL-6.

### EXAMPLES

The examples below are intended to be purely exemplary of the invention and should therefore not be considered to limit the invention in any way. The following examples and detailed description are offered by way of illustration and not by way of limitation.

### Example 1

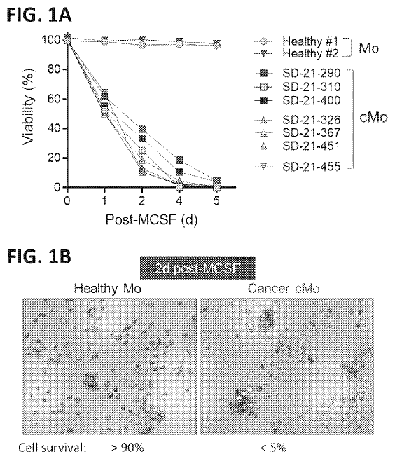

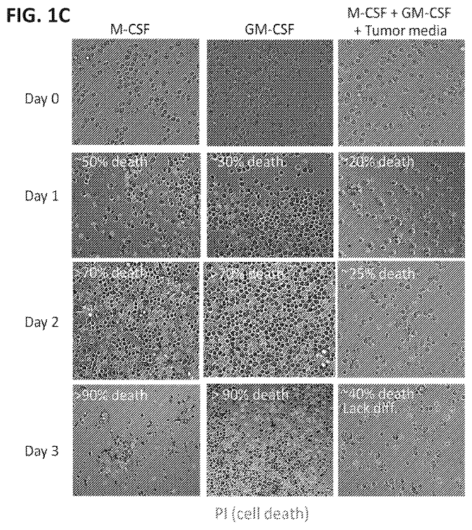

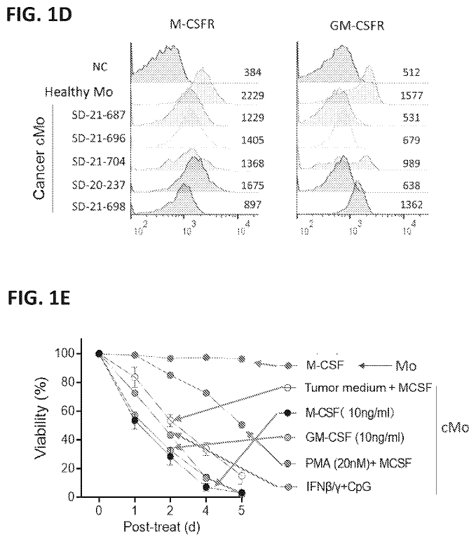

The experiments described herein studying peripheral monocytes from cancer patients with late-stage malignant tumors, e.g., stage II, III or IV with inoperable tumors and/or metastases, found that these monocytes (termed cancer monocytes or cMo) display different responses to the classical macrophage/DC-differentiation factors M-CSF and/or GM-CSF compared to monocytes from healthy donors (healthy monocytes or Mo). In cases of differentiation to macrophages (FIGS. 1A-1C), Mo are readily driven by M-CSF and differentiate into macrophages in 4-5 days, whereas cMo under the same treatment exhibit non-responsiveness (without cell adhesion or morphology change), followed by cell death in 2 days. Similarly, cMo poorly respond to GM-CSF alone or GM-CSF plus IL-4 that drives DC differentiation (data not shown). Over twenty cMo samples from cancer patients with various solid tumors, such as lung cancer, renal cancer, liver cancer, colorectal cancer, thymus cancer, and sarcoma, have been tested and in all cases, cMo consistently displayed non- or poor responsiveness to M-CSF and GM-CSF, but high rates of cell death (FIG. 4). Parallel experiments testing the same M-CSF and GM-CSF reagents successfully differentiated Mo from healthy donors with >90% survival rates.

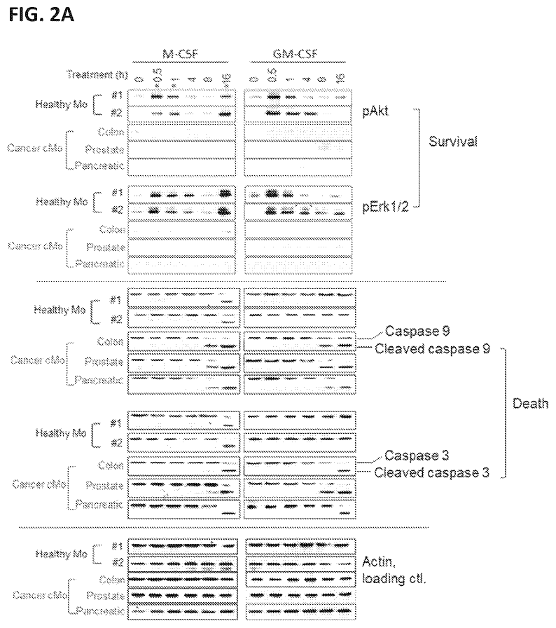

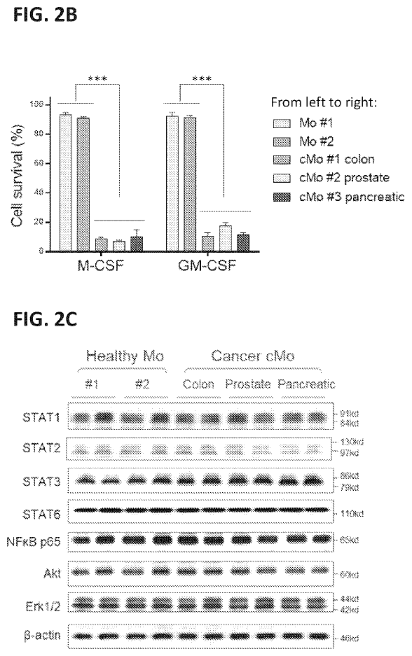

Further studies of M-CSF- and GM-CSF-mediated signal transduction, as shown in FIG. 2A, confirmed that M-CSF and GM-CSF treatments induced strong survival signal transduction in Mo cells via activation of Akt and Erk1/2 (i.e., increased pAkt and pErk1/2 levels compared to total Akt and Erk1/2 protein levels, respectively); however, this signal transduction event was not replicated in cMo. Instead, in response to M-CSF and GM-CSF, cMo exhibited enhanced apoptotic signals and increased caspase-9 and caspase-3 cleavage, leading to cell death (FIG. 2B). Examination of Akt and Erk1/2, as well as other signaling molecules, detected similar total levels in Mo versus cMo (FIG. 2C), ruling out the absence of protein phosphorylation due to reduction of protein translation.

Examination of cell surface cytokine receptors revealed cMo compared to Mo expressing reduced levels of MCSF-R and GMCSF-R (FIG. 1D), partially explaining the poorer responses of cMo to these differentiation factors by implying that the insufficient stimulation strength not only incapacitates cell survival signaling, but also triggers apoptosis. The facts that cMo poorly respond to M-CSF and/or GM-CSF have also been reported by others (Gordon and Freedman, 2006; Ramos et al., 2012) and their results are consistent with the data presented herein. Previous transcription profiling analyses by different groups have also demonstrated varied gene expression patterns between cMo (or termed TEMo) and Mo from healthy donors (Bergenfelz et al., 2015; Cassetta et al., 2019; Chittezhath et al., 2014; Ramos et al., 2020).

### Example 2

Despite failure of differentiating cMo with M-CSF and GM-CSF, it was found that the conditioned medium of TCR ligation-activated T cells induced cMo differentiation into phagocytic APCs.

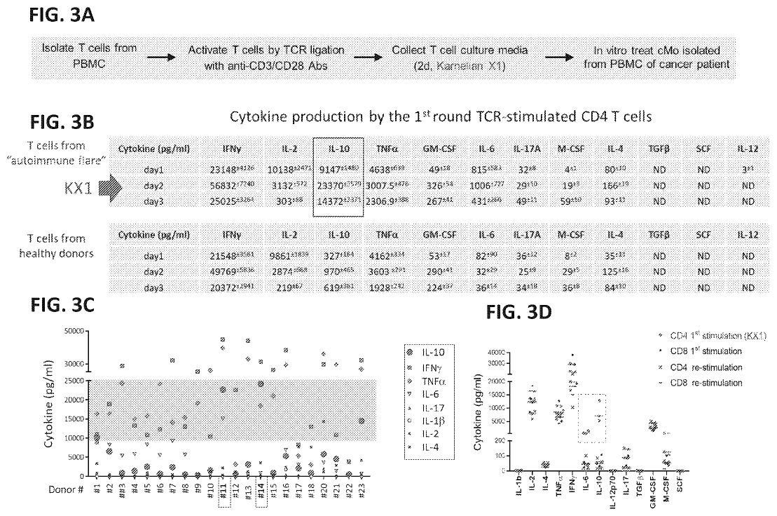

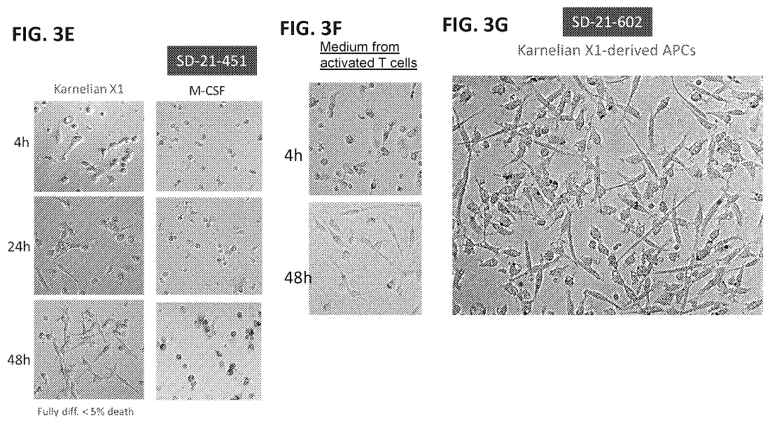

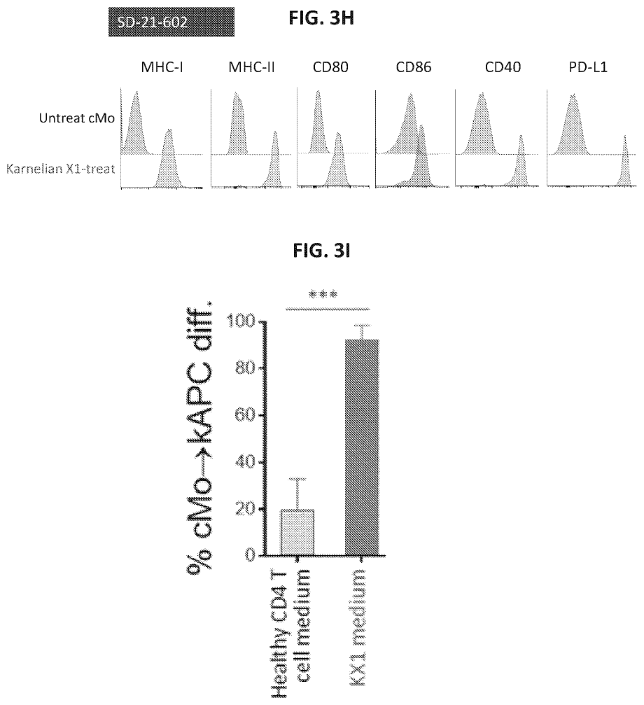

In these experiments (see FIG. 3A), freshly isolated T cells from PBMC from either healthy donors or autologous cancer patients were ligated with anti-CD3 and anti-CD28 antibodies to trigger TCR activation in “autoimmune” CD4 T cells, which secreted abnormally high level of IL-10 (>20 ng/ml) during the first round of anti-CD3/anti-CD28 co-stimulation (FIGS. 3A-3D). After two days, the T cell culture medium was used to treat cMo. In all experiments with cMo from various cancer patients (FIG. 4), the medium from TCR-activated T cells rapidly induced cMo adhesion to the culture dish (1-4 h). The high level of IL-10, together with other cytokines (e.g., IFNγ, TNFα, IL-4, GM-CSF, and IL-6) produced by the same CD4 T cells, was found to be capable of supporting cMo survival and differentiation into a unique type of APC (termed κAPC) as shown by the change in cell morphology (FIG. 3E-3G). Further, this culture condition induced their phenotypic differentiation into professional APCs (FIG. 3I). In two days, cMo-differentiated APCs exhibited increased expression of the cell surface antigen presentation machinery including high level MHC-I, MHC-II, co-stimulatory molecules CD80 and CD86, and APC activation molecules CD40 and PD-L1 (FIG. 3E, the high expression of MHC-II marked differentiation into professional APCs).

Together, these results suggest that certain factors produced by the TCR-stimulated T cells were capable of both supporting cMo survival (adhesion) and driving cMo differentiation into professional APCs. The TCR-stimulated T cell medium harvested on day 2 was termed Karnelian X₁ (or KX1).

### Example 3

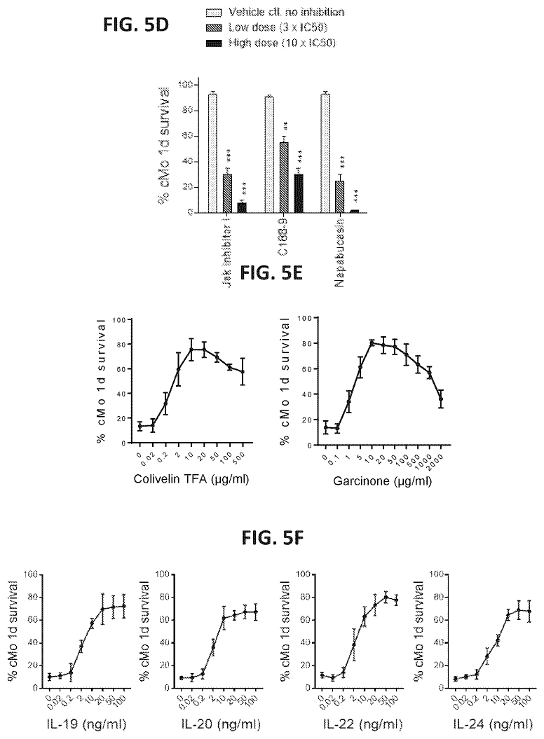

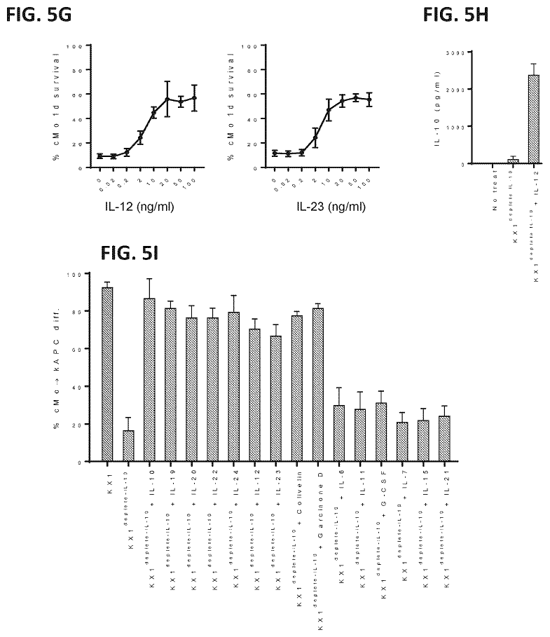

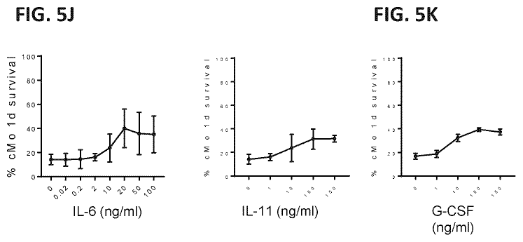

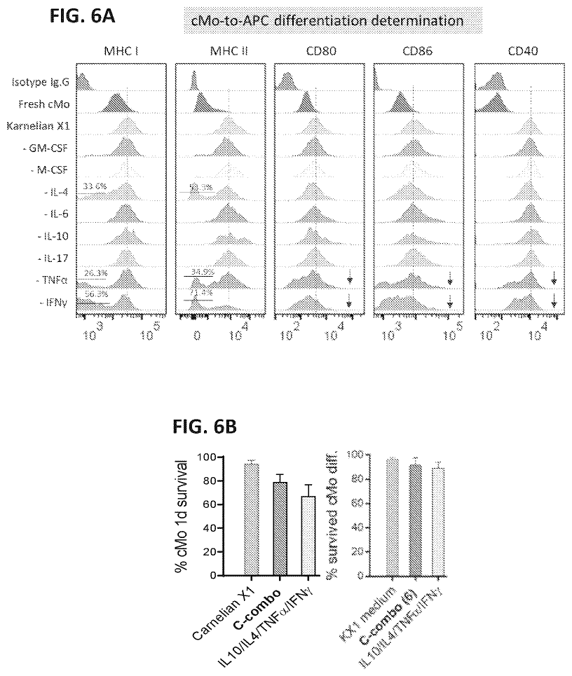

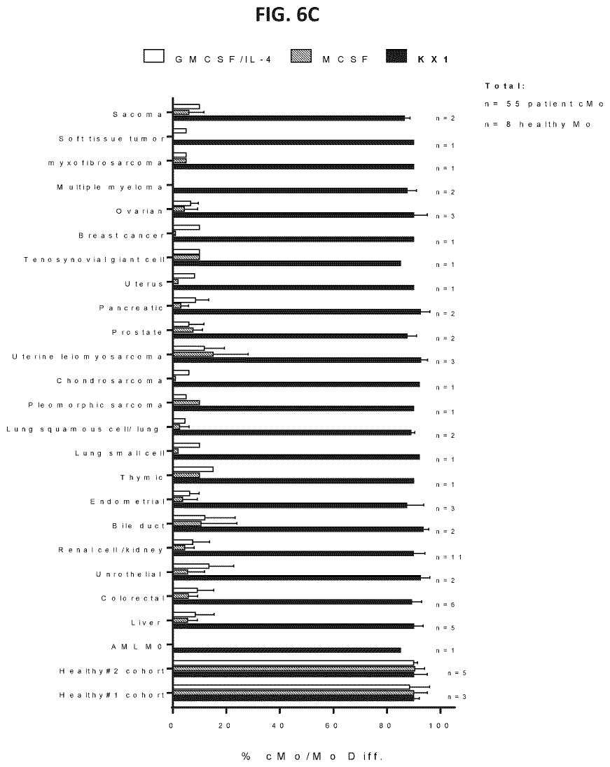

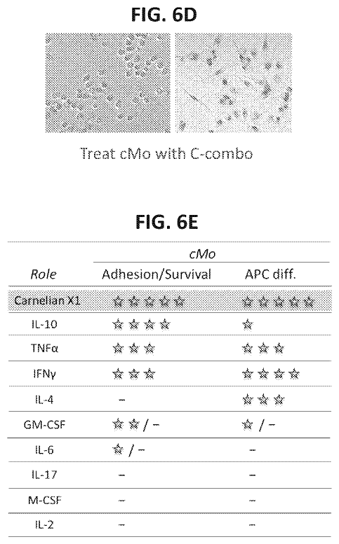

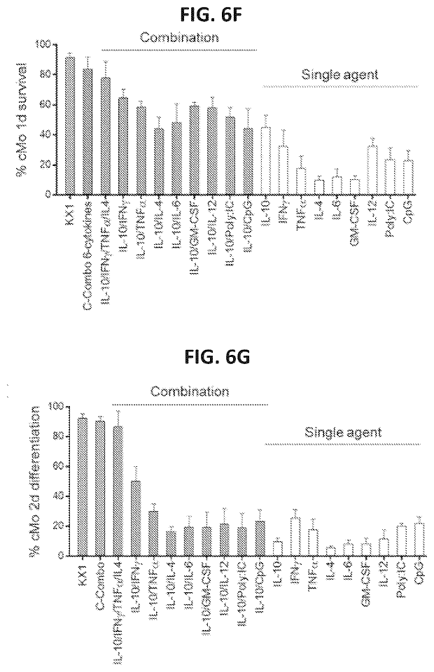

Further studies of Karnelian X₁ found that the biological components capable of supporting cMo survival and differentiation reside in the fraction with molecular weight (MW) between 30 KD-100 KD, a fraction that was enriched with T cells-produced cytokines while excluding exosomes and lipid vesicles. Analyses of cytokines identified IFNγ, IL-2, IL-10, TNFα, GM-CSF, IL-6, IL-17, M-CSF, and IL-4 (FIG. 5A). Particularly, high levels of IL-10 and IL-6 were detected in Karnelian X₁. Further cytokine depletion assays confirmed the critical role of IL-10, and its depletion largely (60-80%) diminished the capacity of cMo adhesion and survival (FIG. 5B). Addition back of IL-10 (recombinant form) to IL-10-depleted Karnelian X₁ dose-dependently restored its survival capacity (FIG. 5C). Inhibition of IL-10R-associated JAK by JAK inhibitor I, or inhibition of the downstream STAT3 by inhibitors C188-9 and Napabucasin, dose-dependently nullified the effect of KX1 (FIG. 5D), resulting in cMo apoptosis. On the contrary, supplementation of STAT3 activators Colivelin TFA or Garcinone D into IL-10-depleted KX1 medium (KX1^{lowIL-10}) restored cMo survival, facilitating cMo differentiation into κAPC (FIG. 5E). A survey of other cytokines that activate STAT3 found that IL-10 family members, e.g., IL-19, IL-20, IL-22 and IL-24, and IL-12 family members (IL-12 and IL-23) could replace IL-10 to support cMo survival and differentiation (FIG. 5F and FIG. 5G, respectively). The mechanism of IL-12 involves induction of IL-10 (FIG. 5H); further, IL-12 is known to directly activate STAT3. Despite the IL-6 family cytokines activating STAT3 through the receptor gp130, treating cMo with IL-6 or its family member IL-11 only moderately supported cMo survival and differentiation (FIG. 5J). Treatment with G-CSF with IL-10-depleted KX1 medium (KX1^{lowIL-10}) showed similar levels of cMo survival to IL-6 treatment (FIG. 5K). However, IL-10 appeared to only weakly influence cMo differentiation to APCs (FIG. 6E), as the remnant cells that survived IL-10 depletion (5-20%) proceeded differentiation to adopt a DC-like morphology and increased expression of MHC-II and the antigen presentation machinery.

IL-10/IL-10R signaling activates the downstream STAT3 signaling pathway. Other cytokines in the IL-10 family can activate STAT3 and were further shown to induce similar levels of cMo survival in vitro. Similarly, IL-12 and IL-23 are STAT3 activators (e.g., through activating IL-10 production and signaling in the cMos), and both cytokines showed an increase in cMo survival at rates higher than IL-6 but lower than IL-10. IL-6 is a known activator of the STAT3 pathway, however the cMo survival rates were lowest when incubated with IL-6 alone, thereby highlighting the unexpected results of IL-10, IL-12, and their cytokine family members as a pro-survival mechanism for cMos in vitro. Small molecules that activate STAT3 showed similar pro-survival efficacy as IL-10. See Table 1 below.

TABLE 1
STAT3 activators that promote cMo survival in vitro.

| | | Optimal | Effectiveness |
|---|---|---|---|
| | | concentration | (cMo survival |
| STAT3 activator | Name | (ng/mL) | rate) |
| IL-10 family | IL-10 | >10 ng/mL (10-100) | 90-100% |
| cytokine | IL-22 | >10 ng/mL (10-100) | 80-100% |
| | IL-19 | >10 ng/mL (10-100) | 70-85% |
| | IL-20 | >10 ng/mL (10-100) | 70-85% |
| | IL-24 | >20 ng/mL (20-100) | 70-85% |
| IL-12 family | IL-12 | >10 ng/mL (10-100) | 50-70% |
| cytokine | IL-23 | >10 ng/mL (10-100) | 50-70% |
| IL-6 family | IL-6 | 20-100 ng/mL | 20-30% |
| cytokine | | | |
| Small molecule | Colivelin TFA | 10-100 μg/mL | 90-100% |
| | Garcinone D | 0.5-2 μg/mL | 90-100% |
| G-CSF | G-CSF | >1 ng/mL (1-200) | 30-40% in |
| (in IL-10-depleted | | | IL-10- |
| KX1) | | | depleted KX1 |
| IL-7, -9, -15, | IL-7, -9, -15, | >10 ng/mL (10-200) | 10-20% |
| and -21 (in | and -21 | | |
| IL-10^{-depleted} | | | |
| KX1) | | | |

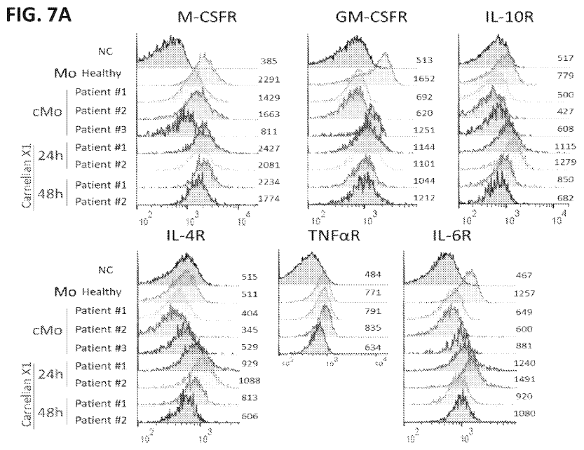

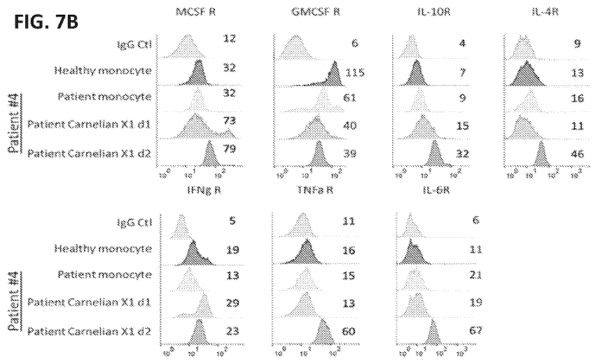

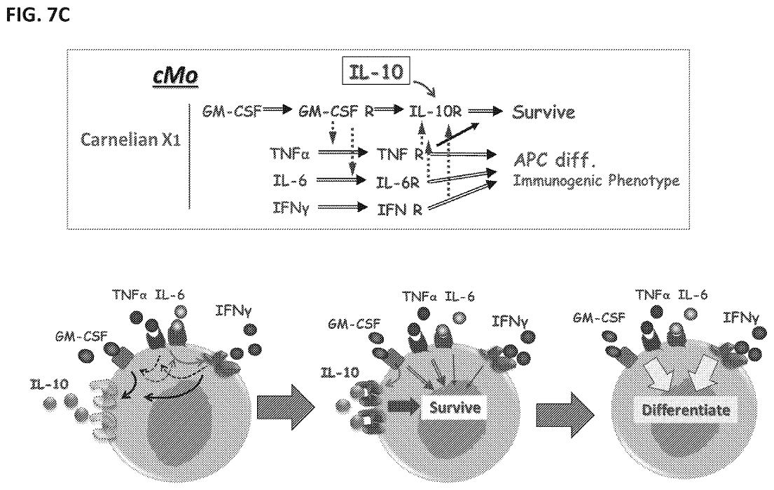

Three other cytokines, TNFα, IFNγ and IL-4, were also found to be important. Depletion of TNFα or IFNγ in Karnelian X₁ reduced both cMo survival (˜20%) and survived cMo differentiation into APCs (30-70% reduction of MHC-II) (FIG. 5B). IL-4, on the other hand, did not affect cMo survival/adhesion but significantly affected cMo differentiation to APCs (˜50% reduction, FIG. 6A). The differentiation of cMo from >100 cancer patient samples was tested. These patients are with different stages of cancer, including both liquid leukemia and solid tumors, and in all cases, KX1 robustly differentiated cMo into κAPC (FIGS. 6C-6D). FIG. 6E summarizes the roles of each cytokine in Karnelian X₁ as defined by detailed experiments. Among other cytokines, IL-2, IL-17 and M-CSF were dispensable, while the roles of GM-CSF and IL-6 were variable. Experiments found that GM-CSF and/or IL-6 were important for cMo survival and differentiation in some cases, while exhibiting dispensable in others. These inconsistencies, together with the incompletely synchronized responses of cMo to Karnelian X₁ after depletion of IL-10 or other cytokines, and later studies revealing varied cytokine receptor expression among cMo obtained from different patients (see FIGS. 7A-7B), suggest that cMo represent populations of heterogenous, stage-variable monocyte-lineage cells with status (protein expression and functionality) influenced by in vivo cancer types and disease status. This fact of cMo contrasts to Monocytes from healthy donors that generally display nearly unified receptor expression and responses to treatments.

Given the above studies, a cytokine cocktail, termed C-combo™, was formed with the compositions comprising important components in Karnelian X₁ (also referred as to “KX1” or “Carnelian X₁”) (IL-10, IL-4, IFNγ, TNFα, GM-CSF, and IL-6). This KX1 and the KX1 shown in FIG. 3B exhibit comparable results for the various experiments conducted herein. Testing C-combo, as well as other cytokine combinations, confirmed its ability to support cMo survival/adhesion and differentiation into APCs (FIGS. 6B, 6D, and 6F-6G).

### Example 4

Despite that IL-10 is critical for Karnelian X₁ to derive cMo differentiation into APCs, detection of cytokine receptors found that freshly isolated cMo often expressed no or low-level IL-10R. Treating cMo with Karnelian X₁ rapidly and transiently increased IL-10R expression (FIGS. 7A-7B). Similarly, freshly isolated cMo expressed no/low IL-4R, which increased shortly after Karnelian X₁ treatment. Differently, variable levels of receptors for IFNγ, TNFα, GM-CSF, and IL-6 were detected on cMo prior to Karnelian X₁ treatment, suggesting that these cytokines likely acted as the first line stimuli and triggered increases in IL-10R and IL-4R and later chain regulations. These results, together with our previous findings in murine tumor models (Bian et al., Eur J Immunol. 2018 June; 48 (6): 1046-1058.), postulate that IFNγ, TNFα, GM-CSF, IL-6, and/or other possible factors in Karnelian X₁, mediate signal transduction via their receptors and induce IL-10R and IL-4R expression, which then enables IL-10 to initiate cMo adhesion and APC differentiation together with IFNγ and TNFα. The parallel IL-4R signaling with IFNγ and TNFα consequently promotes the expression of antigen presentation machinery and the full differentiation to professional APCs. The proposed mechanisms are depicted in FIG. 7C.

The results that cMo from different patients express varied levels of cytokine receptors for M-CSF, GM-CSF, IFNγ, TNFα, and IL-6 suggest that cMo are heterogeneous monocyte-lineage cells. The protein expression profiles of cMo are likely associated with cancer types and disease stages that significantly impact on bone marrow myelopoiesis and monocyte maturation. As shown previously (Zhen, et al, 2019), tumor conditions influence monocyte-lineage development and upregulate CCR chemokine receptor expression, leading to the release of ‘immature’ monocytes from bone marrow into circulation.

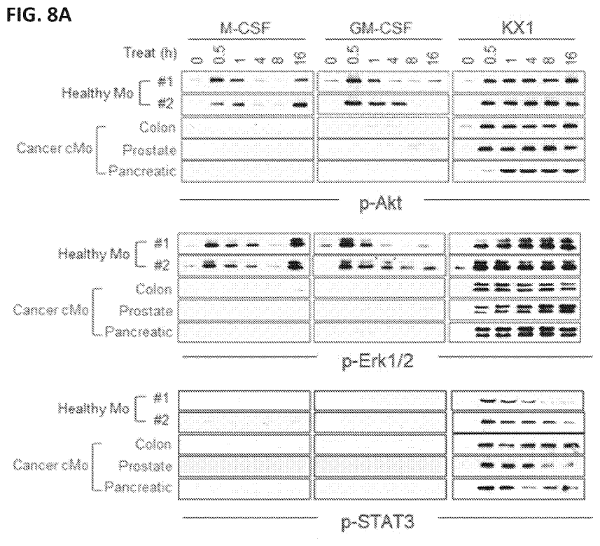

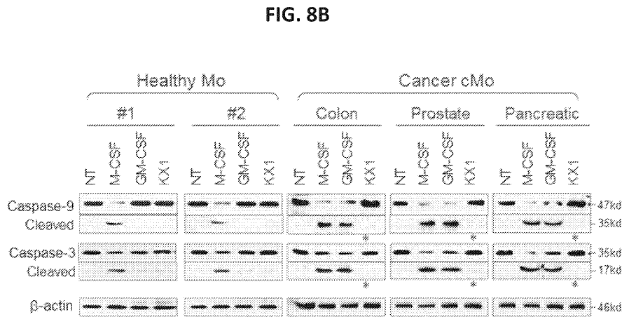

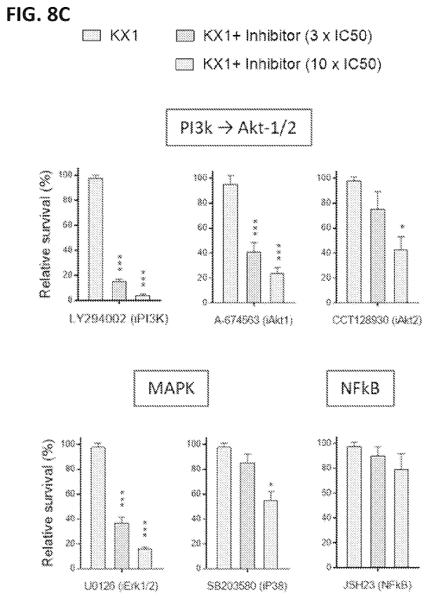

The mechanisms by which KX1 could promote cMo survival was further studied. In contrast to M-CSF and GM-CSF, both of which failed to activate Akt and Erk1/2 but did induce apoptotic signals via cleavage of caspase-9 and caspase-3, KX1 treatment of cMo induced strong Akt and Erk1/2 activation (p-Akt and p-Erk 1/2) without inducing apoptosis-related caspase-9 and -3 cleavage (FIG. 8A-8B). In addition, KX1, especially via its key component IL-10, strongly activated STAT3, which is another pro-survival signal (FIG. 8B). Testing pharmacological inhibitors confirmed that PI3-Akt1/2 and MAPK signaling pathways were involved in KX1 mechanisms; inhibition of PI3K (LY294002), or Akt1 (A-674563) or Akt2 (CCT128930), or the MAPK member Erk1/2 (U0126) abrogated KX1 effectiveness (FIG. 8C).

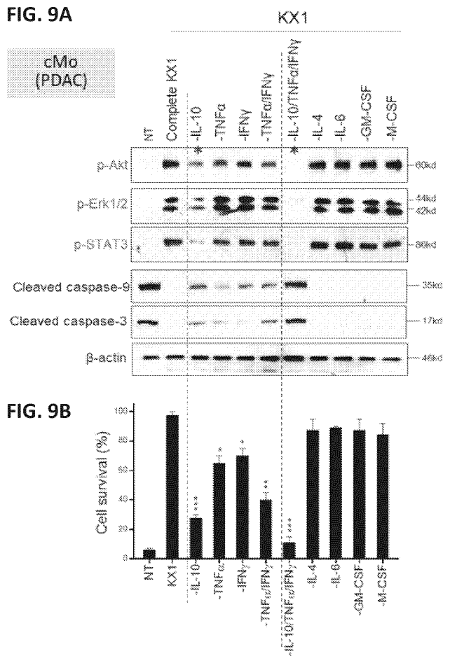

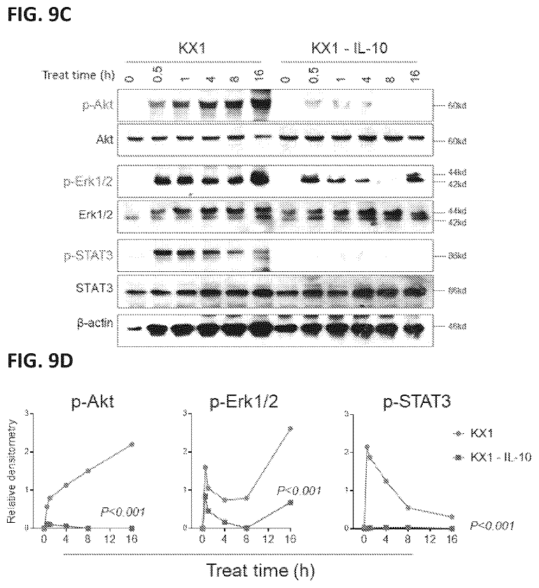

The role of IL-10 and other key factors in KX1 was further studied. As shown in FIG. 9A-9D, depletion of IL-10 diminished (>70%) KX1-mediated activation of Akt, Erk 1/2, and STAT3, and this global reduction of survival signal was associated with an increase in cell death signal through activation of caspase-9 and caspase-3. The role of IL-10 alone was superior to TNFα plus IFNγ, of which depletion still preserved a significant level (˜50%) of Akt, Erk 1/2, and STAT3 activation. Triple depletion of IL-10, TNFα, and IFNγ completely nullified the impact of KX1 on supporting cMo survival and on driving differentiation into κAPC. In conclusion, these three cytokines, especially IL-10, were the core functional components of the KX1 recipe.

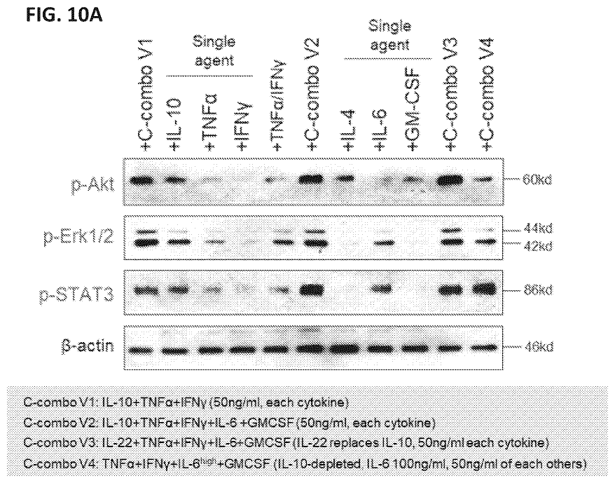

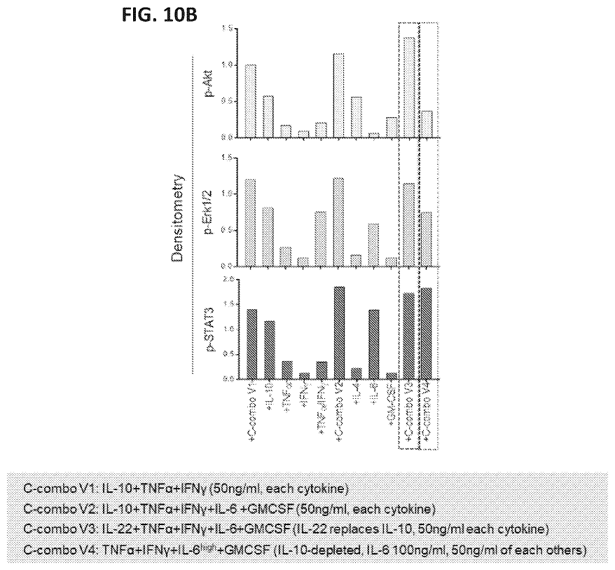

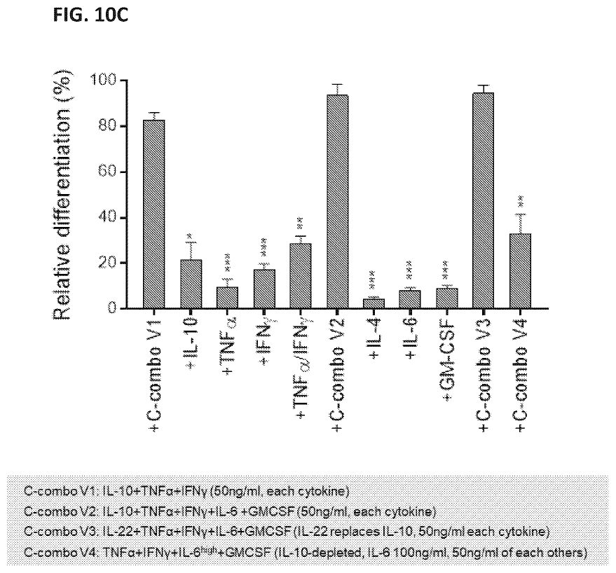

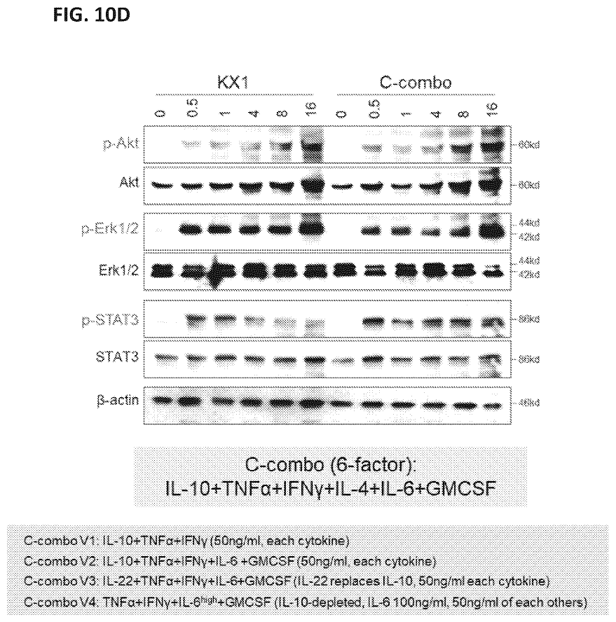

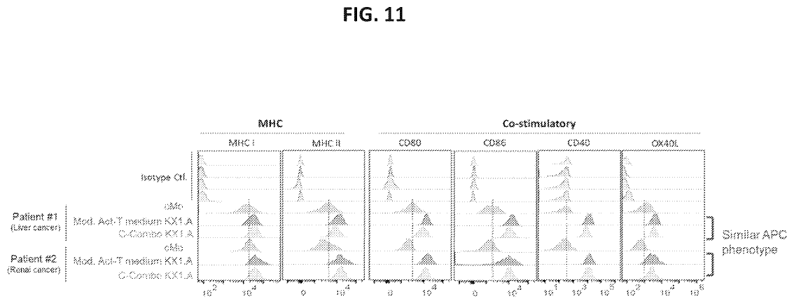

As shown in FIG. 10A-10C, triple cytokine combination (C-combo V1) was capable of achieving >80% cMo differentiation, although each component alone was not sufficient. Further addition of IL-6 and GM-CSF moderately enhanced effectiveness (C-combo V2). These studies also confirmed that IL-10 can be replaced by a member of the same interleukin family, e.g., IL-22 (C-combo V3), but not by IL-6 (C-combo V4) despite the latter being an activator of STAT3. Compared to IL-10 or IL-22, IL-6 exhibited weaker ability to activate Akt in cMo. Studies of the current recipe of six cytokine C-combo (IL-10, TNFα, IFNγ, IL-4, IL-6, and GM-CSF) confirmed its ability to activate Akt, Erk1/2, and STAT3 in a manner similar to the originally identified KX1 (IL-10high, activated CD4 T cell medium) (FIG. 10D). Furthermore, analysis of the κAPC profile differentiated from human patient cMo (i.e., patients with liver and renal cancers) using KX1 with IL-10 (i.e., KX1.A) showed increased markers of antigen presentation machinery (FIG. 11).

### Example 5

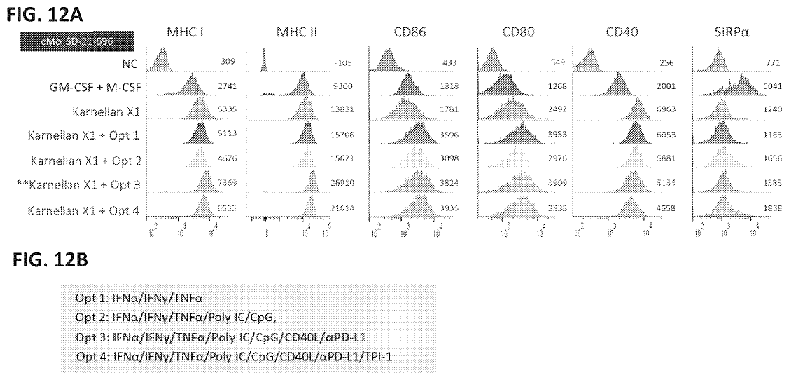

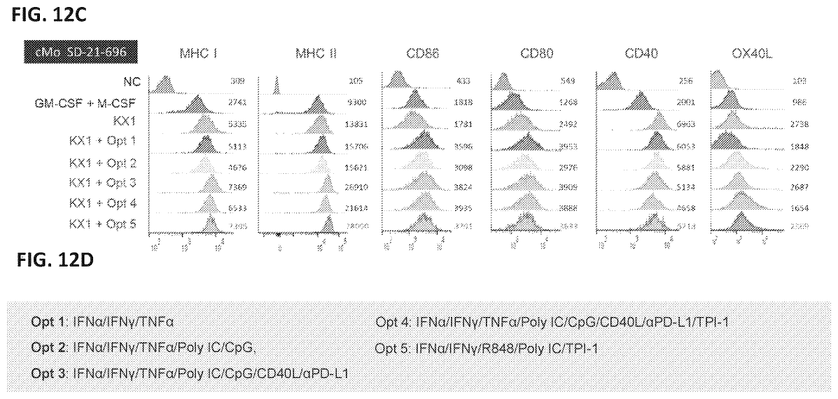

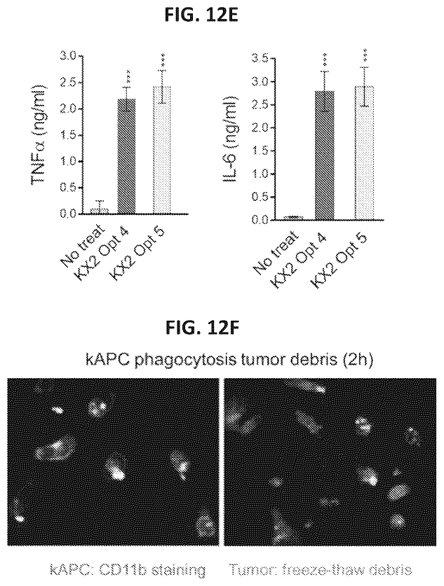

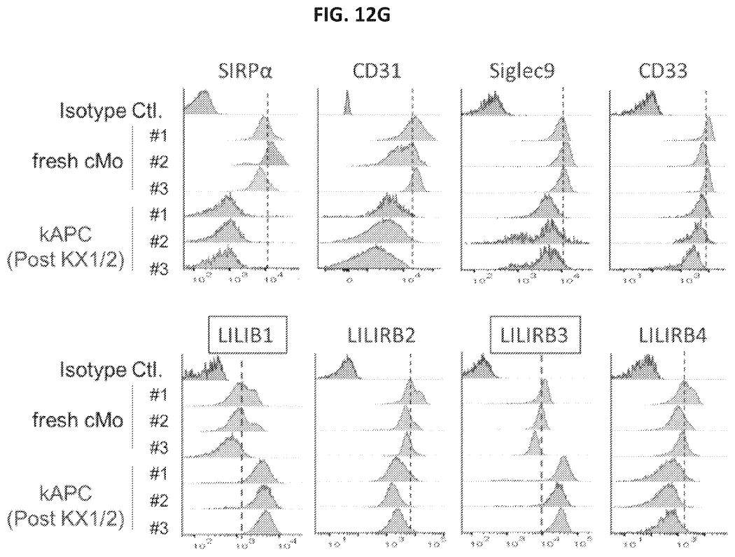

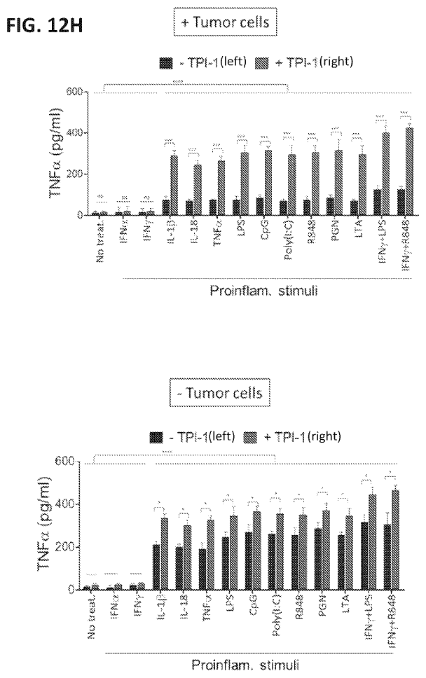

Karnelian X₁ cytokines, additional cytokines, TLR ligands and other factors for phenotypic optimization of cMo-derived APCs were further tested. These efforts led to the formation of Karnelian X₂, which acted to further bolster κAPC for phagocytosis, immunogenic antigen presentation, and T cell priming, and also endowed κAPC with a proinflammatory phenotype and the ability to resist repolarization in the tumor microenvironment. KX2™ comprises cytokines (choices of IFNα, IFNγ and TNFα), TLR ligands (choices of Poly IC, CpG, R848), and TPI-1. Particularly, Karnelian X₂ allows to maximally support cMo differentiation into highly proficient, immunogenic APCs (FIGS. 12A-12D). The final κAPC cell product displays high expression of the immunogenic antigen presentation machinery (MHC-I/II, CD80/86, CD40, OX40L), proinflammatory phenotype (TNFα and IL-6), high capability of phagocytosis, and reduced expression of most inhibitory receptors (SIRPα, LilRB, Siglec), as shown in FIGS. 12A-H. In FIG. 12H, the KX2 component TPI-1 is a SHP-1 inhibitor that promotes proinflammatory phenotype of κAPC in the presence of tumor cells.

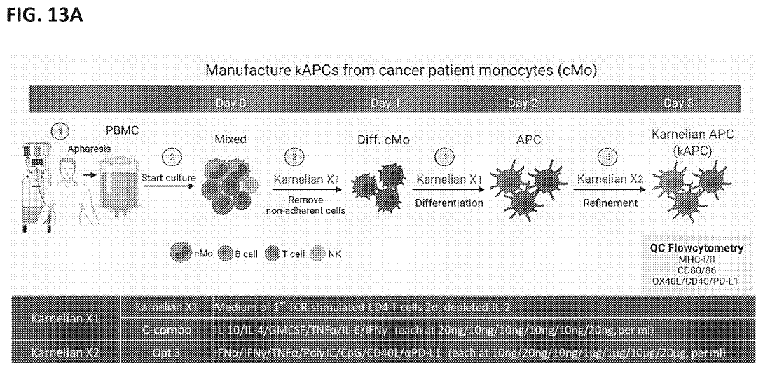

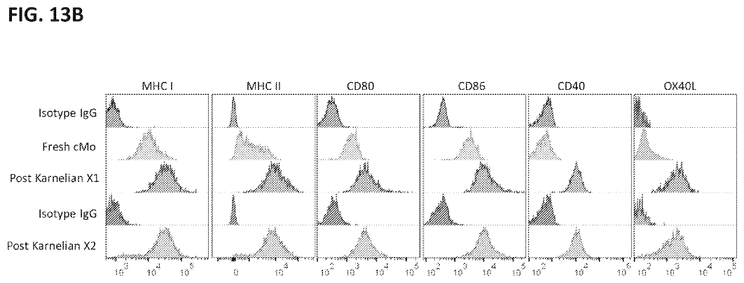

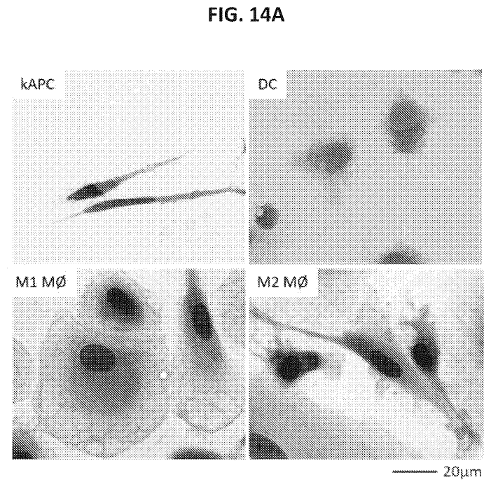

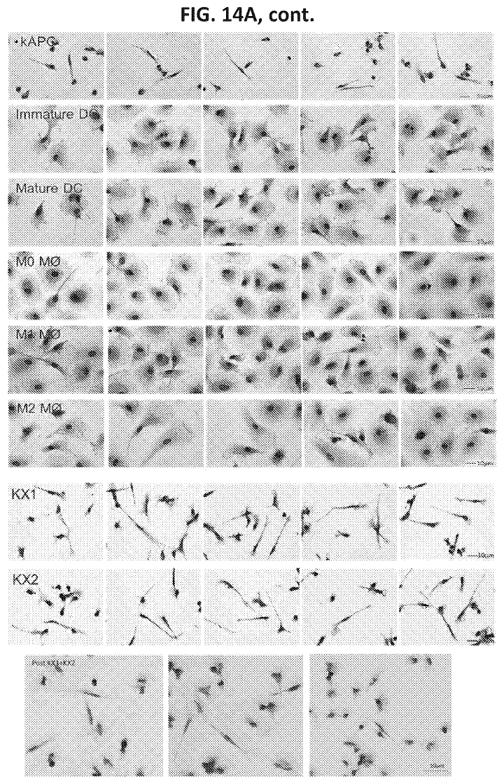

An in vitro two-step APC engineering procedure was thus established including the first step Karnelian X₁/C-combo treatment that differentiates cMo to APC, followed by the second step of refinement with Karnelian X₂ (FIG. 13A), which optimizes APCs by promoting a strong immunogenic phenotype and high expression of MHC-I to ensure potent priming of tumor-specific CD8 T cells that are essential for tumor cytotoxicity in vivo. Exemplary QC1 data are provided in FIG. 13B. The final product of APCs, termed Karnelian patient-derived APC or κAPC, are further developed for APC-based cancer vaccines and cell therapy and APC-extended Neo-T therapy.

The κAPC cell product is distinct, for example the cell morphology. cMo- or Mo-derived κAPC display smaller sizes, spindle, elongated or multi-shaped cells compared to Mo-derived DC and macrophages (FIGS. 14A and 14B). In culture, κAPC adhere to the matrix substratum but can be easily dislodged by repeated pipetting or brief trypsinization (<2 min at 37° C.). In comparison, Mo-derived DC (GM-CSF/IL-4) generally are not or are only loosely adherent, whereas Mo-derived macrophages (M-CSF) strongly adhere to matrix and require long trypsinization (≥10 min, 37° C.) to dislodge.

The κAPC cell product showed high levels of antigen presentation. Monocytes from healthy donors (Mo) were induced to κAPC by KX1 with IL-10 and KX2. Mo were also induced to differentiate into MoDC and macrophages. In brief, Mo were treated with GM-CSF and IL-4 at 10 ng/ml each for 5 days; then MoDC were treated with LPS at 100 ng/ml for 18 hrs for DC maturation to increase expression of antigen presentation machinery. Macrophages (MØ) were skewed towards the proinflammatory M1 phenotype, which is associated with high expression of antigen presentation machinery. MØ were also skewed towards the anti-inflammatory M2 phenotype. M1 MØ were produced by treating Mo with M-CSF at 10 ng/ml for 5 days followed by LPS at 100 ng/ml and IFNγ at 20 ng/ml for 18 hrs. M2 MØ were produced by treating Mo with M-CSF at 10 ng/ml for 5 days followed by LPS at 100 ng/ml and IL-4 and IL-10 at 20 ng/ml each for 18 hrs. κAPC were produced by treating Mo with KX1 for 2 days followed by KX2 for 18 hrs.

Flow cytometric analyses were performed to examine and compare cell surface antigen presentation machinery between κAPC, MoDC (mature APC), and M1 and M2 MØ (FIG. 15A). These studies revealed that κAPC are equipped with highly proficient, immunogenic antigen presentation capacity, especially with higher expression of costimulatory molecules such as CD80, CD86 and CD40 compared with MoDC and M1 MØ, both professional APCs. Particularly, it was found that κAPC compared to MoDC or MØ express exceptionally high level of CD40, a molecule facilitating CD8 T cell activation during antigen presentation.

In parallel, cMo from cancer patients (e.g., patients with inoperable prostate cancer, colorectal cancer and pancreatic cancer) were induced to κAPC by KX1.A and KX2. Analyses of κAPC antigen presentation machinery found similarly upregulated MHC-I/II, CD80/86, CD40, OX40L, ICOSL, and CD70 costimulatory molecules. The co-inhibitory molecule CD31 was reduce, while PD-L1 level was elevated (FIG. 15B).

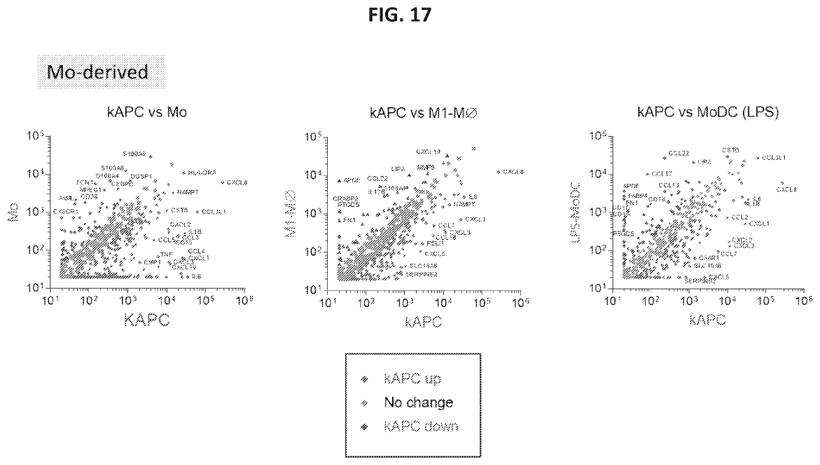

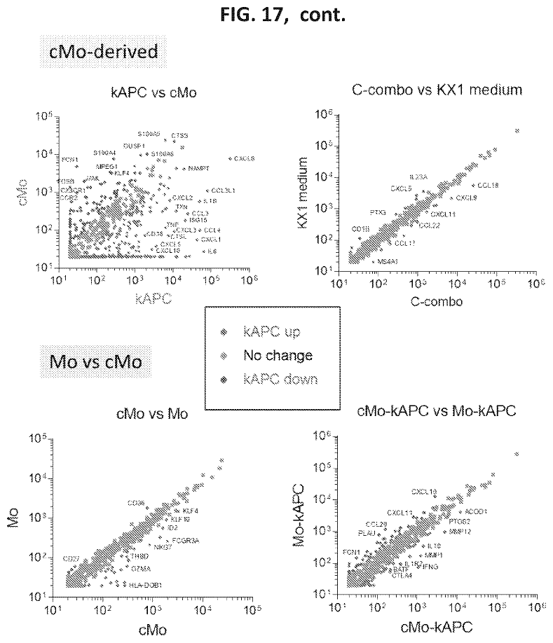

The κAPC cell product showed unique a gene signature that was similar between κAPC cells derived from healthy individuals and cancer patients. The data indicate that, regardless of monocytes (Mo or cMo) from different healthy donors and cancer patients, KX1/2-differentiated κAPC displayed similar gene signature, suggesting that KX1/2 mediated similar signaling mechanisms to drive cMo/Mo differentiation (FIG. 16A). Additional transcriptional analyses were performed comparing Mo versus cMo and their derived κAPC, as shown in FIG. 16B. Moreover, transcriptional analyses were performed comparing cMo-derived κAPC using either Modified Act-T medium (KX1 medium) or C-combo, both comprising IL-10 as a STAT3 activator. Similar gene transcription profiles were obtained in κAPC produced with either medium, as shown in FIG. 16C. Comparison of gene signatures in κAPC to MoDC and M1 MØ identified a number of transcripts that differed between κAPC versus MoDC and M1 MØ (see FIG. 17).

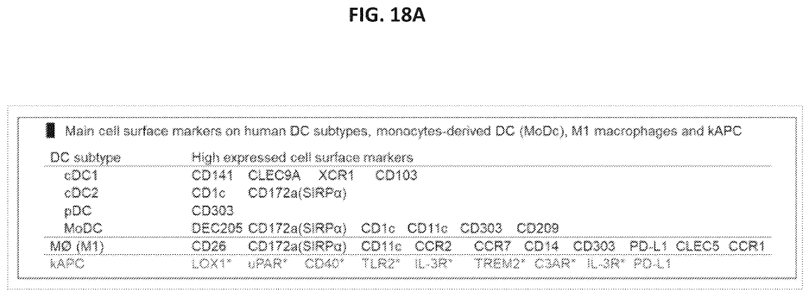

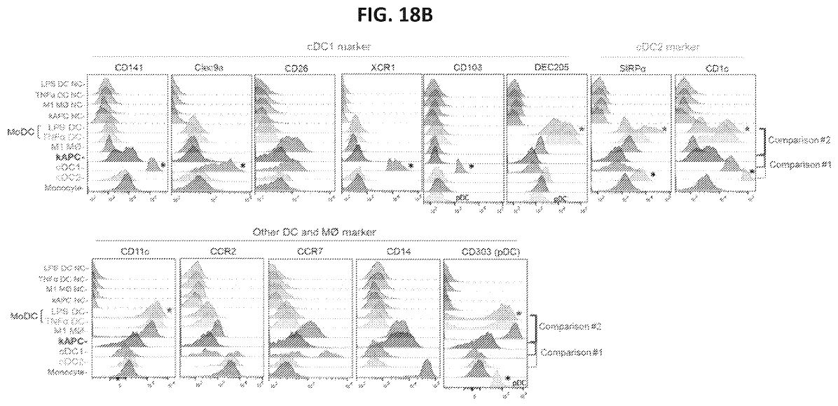

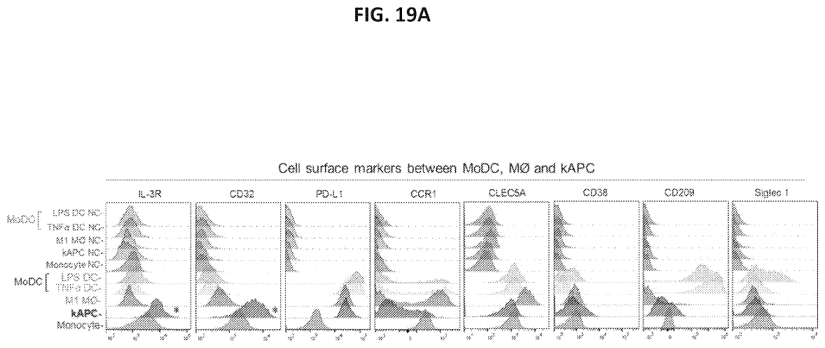

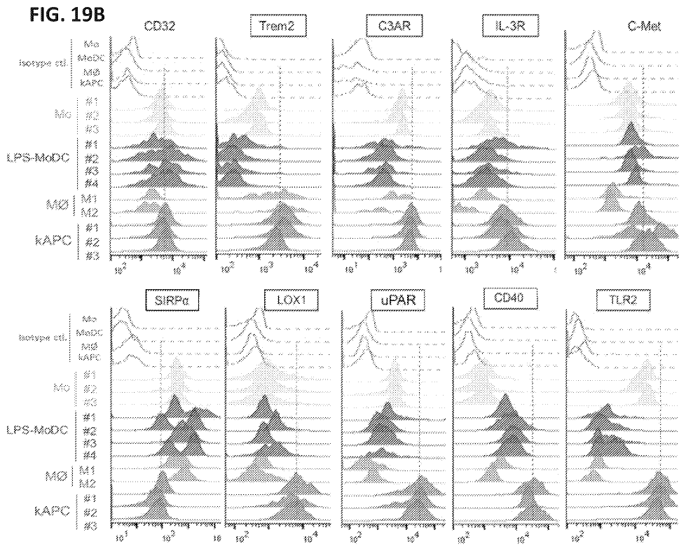

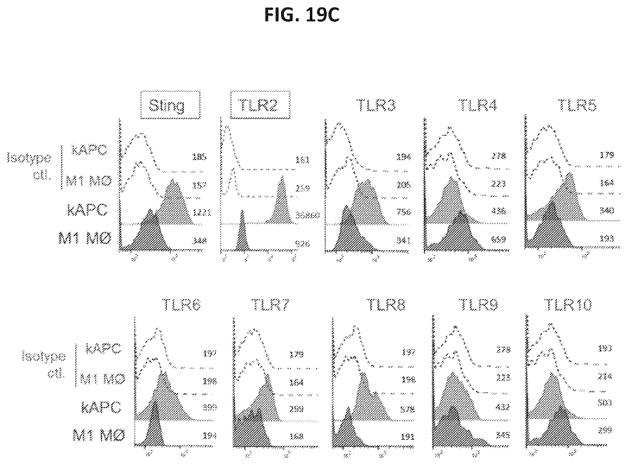

The transcriptional profiles or gene signatures revealed that κAPC express unique genes compared to mature, LPS-treated MoDC and proinflammatory macrophages (M1 MØ), both of which are APCs. The levels of multiple proteins that are coded by these variably expressed genes and could impact APC phenotype were examined by flow cytometry. These studies identified a set of proteins uniquely expressed on κAPC, thus providing cell gene signatures that separate κAPC from cDC1, cDC2, pDC, and macrophages, as summarized in FIGS. 18A-18B and 19A-19C.

In particular, κAPC highly expressed LOX1, uPAR, CD40, TLR2, IL-3R, C3AR, and PD-L1 on the cell surface (FIG. 18B). Except for PD-L1, which was also highly expressed on other APCs such as mature MoDC and M1 MØ, other molecules exhibited a unique pattern in κAPC. κAPC did not express specific markers found in cDC1, cDC2 and pDC, nor expressed markers of MoDC (FIG. 18B). Thus, κAPC do not belong to the DC antigen presenting cell category. Moreover, κAPC also displayed varied protein expression profiles compared to macrophages of either M1 or M2 phenotype (FIGS. 18A-18B and 19A-19C).

In addition, examination of pattern recognition receptors (PRR) found that κAPC expressed most TLRs and, in particular, highly expressed STING, TLR2, TLR3, and TLR8 (FIG. 19C). These data suggest that κAPC are keen sentinel/responsive cells, likely most capable of sensing for cell damage and for bacterial and viral pathogens. The high expression of LOX1, uPAR, IL-3R, and C3AR receptors on κAPC, together with the high levels of CD40 and PRRs, feature κAPC to be potent proinflammatory leukocytes and antigen presenting cells, suitable for in vivo and in vitro vaccination to activate T cells and provide long-lasting adaptive immunity.

### Example 6

Karnelian X₁/X₂-differentiated κAPC from cMo of cancer patients are found to be excellent anticancer antigen presenting phagocytes, inherently with enhanced proinflammatory signatures and increased expression of an immunogenic antigen presentation machinery. Two lines of autologous immunotherapy strategies are under development with κAPCs, both against cancers (e.g., solid tumors) through in vivo activation of tumor-specific CD4 and CD8 T cell immunity against cancer.

### 1. κAPC Combination Therapies

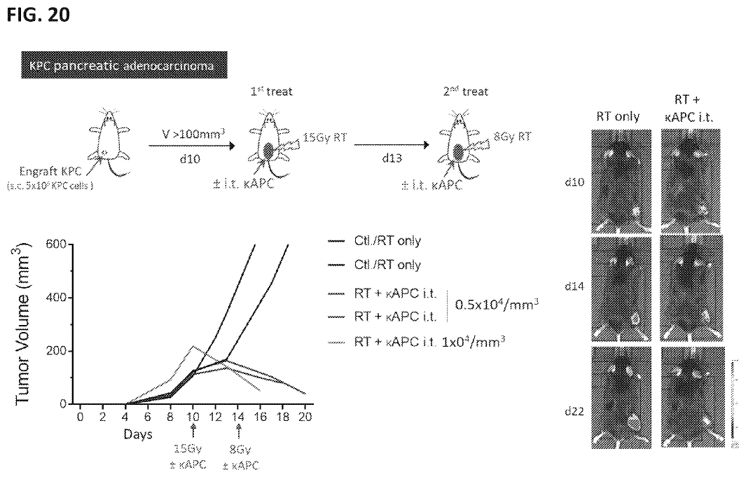

This line of therapies combines κAPCs in vivo administration with regimens that enhance phagocytosis, proinflammation and antigen presentation to achieve high efficacies of phagocytic κAPC-mediated induction of tumor-specific T cell immunity. Specifically, κAPCs can be administrated via routes including but not limited to: intratumoral injection (i.t.), intravenous administration (i.v.), intraperitoneal administration (i.p.), subcutaneous administration (s.c.), intracutaneous administration, intramuscular injection, etc. Combination regimens include those damage tumor cells and produce immunogenic cell death (ICD), including but not limited to: radiotherapy (RT), immune checkpoint inhibitor (ICI), oncolytic virus, cytokine and TLR modulators of the TME, chemotherapy, anticancer antibodies, or kinase inhibitors. FIG. 20 shows an example of κAPC combination with tumor-focal RT against KPC pancreatic ductal adenocarcinoma in a preclinical study.

Specifically, WT mice were subcutaneously (s.c.) engrafted with KPC pancreatic cancer. Treatments started on day 10 post the engraftment when tumors established to >100 mm³. Given that KPC resists RT, control treatments with two cycles of tumor-focal RT (1^{st} 15Gy on d10 and 2^{nd} 8Gy on d14) produced minimal beneficial effects. κAPC combination treatments were conducted following the same two cycles of RT with the addition of κAPC (derived from tumor-bearing mice with methods described herein) intratumoral injection (i.t.) immediately after each RT section (within 1 h). Two doses of κAPC, 0.5×10⁴ and 1×10⁴ cells per mm³ tumor mass, were given via multi-point i.t. injection. As shown, despite RT alone being ineffective, κAPC and RT combination led to durable tumor regression, producing a synergistic effect.

### 2. Therapeutic κAPC Cancer Vaccine (Vs Prophylactic Vaccine)

These studies demonstrate that Karnelian X₁/X₂-differentiated κAPCs are excellent immunogenic antigen presenting cells capable of through MHC-I cross presentation and MHC-II presentation robustly activating tumor antigen-specific CD8 and CD4 T cells, respectively, as well as inducing a long-lasting anticancer memory with cellular immunity and tumor-specific antibodies. These features of κAPCs, and the fair and rapid ex vivo procedure that produces κAPCs to large numbers from cancer patient monocytes, enable κAPCs to be ideal autologous therapeutic cells, especially for establishing anticancer vaccines that both treat cancer and prevent relapses.

### Personalized Total Tumor (TT)-κAPC Vaccine

The first line universal κAPC vaccine uses the total tumor biopsy/cells from the patient as the antigen (Ag). The specific procedure involves in vitro incubating κAPCs with fresh or freeze-thaw tumor biopsy materials, or cultured tumor cells from tumor biopsy, a step for κAPC phagocytosis and obtaining tumor Ags. After antigen processing (6-18 h), these Ag-obtained κAPCs are used to immunize the same cancer patient through either intratumoral injection (i.t.), or intravenous administration (i.v.), or intraperitoneal administration (i.p.), or subcutaneous administration (s.c.), intracutaneous administration, or intramuscular injection.

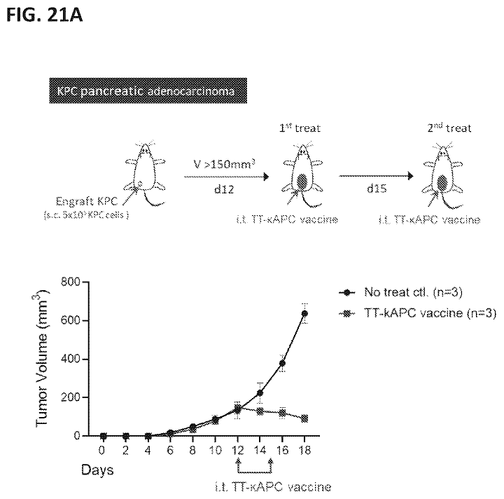

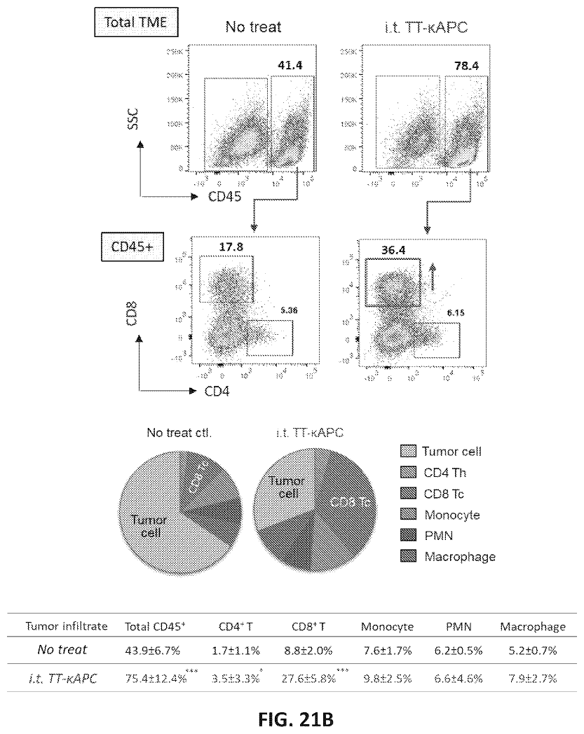

FIG. 21A shows an example of TT-κAPC vaccine established against KPC pancreatic cancer. Murine bone marrow-derived κAPCs were generated ex vivo by Karnelian X₁/X₂ treatment, followed by incubation (about 16 hours) with freeze-thaw KPC cells for phagocytosis of tumor antigens. The KPC-phagocytosed κAPCs (TT-κAPC vaccine) were then i.t. injected into established KPC tumors on day 12 and 15 at the dose 0.5×10⁴ κAPC per mm³ tumor mass.

As shown in FIG. 21A and FIG. 21B, mice treated with TT-κAPC vaccination successfully achieved tumor regression and a higher frequency of various immune cells including total CD45+ immune cells, CD4 T cells, CD8 T cells, macrophages, and monocytes. These results indicate that TT-κAPC vaccination method induces successful immune response against tumor antigens which effectively treats cancer.

Accordingly, advantages of TT-κAPC vaccine include induction of cancer neoantigen-specific T cell immunity and antibodies, strengthening a long-lasting immune memory against cancer, and immunity against fibrosis (pancreatic cancer) and the tumor-supporting TME.

### Personalized Neoantigen Long Peptide (LP)-κAPC Vaccine:

This κAPC vaccine uses synthetic, AI-identified neoantigen-containing LPs from the patient tumor. The procedure of generating the vaccine involves in vitro incubating (pulse) κAPCs with synthetic neoantigen-containing LPs, followed by immunization of the patient. Advantages of LP-κAPC vaccine elevate high cancer specificity while minimizing autoimmunity. This vaccine strategy is especially applicable to cancers that are caused or associated with virus infection, e.g., HPV, EBV, HB/CV, HIV, HHV-8, HTLV-1, MCV, as well as infections by other pathogens and biological agents.

### Shared Tumor-Associated Antigens TAA-κAPC Vaccine:

This strategy explores a series of identified TAA including abnormally expressed ‘self-antigens’, e.g., cancer/testis antigens, differentiation and over-expressed antigens, ‘non-self’ antigens of viral origins, and frequent mutations-caused neoantigens shared in different types of cancer. Examples of the first category include melanoma antigen-1 (MAGE-1), prostate-associated PAP, PSA and PSMA, breast cancer-associated BCAR3, and multi-cancer associated MUC1. Examples in the second category include LMP1/2 associated with nasopharyngeal carcinoma and lymphoma, E6 and E7 proteins of high-risk human papillomavirus (HPV), and retrovirus Tax protein found in adult T cell leukemia. Examples of frequent mutations-caused neoantigens include p53 mutations found in cancers of liver, head neck and colorectal, and KRas mutations found in the cancers of lung, pancreatic, colorectal, etc.

Different from TT- and LP-κAPC vaccines, TAA-κAPC vaccine uses TAA recombinant proteins/peptides or neoantigen-containing LPs that are off-the-shelf. The procedure to generate TAA-κAPC vaccine involves: 1) in vitro production of autologous κAPCs, 2) incubating (pulse) κAPCs with TAA recombinant proteins/peptides or neoantigen-containing LPs, and 3) immunization of the patient.

Advantages of TAA-κAPC vaccine: Biopsy is not required. TAA is off-the-shelf, and only PBMC from the patient is needed.

### DNA/mRNA-κAPC Vaccine

In this strategy, κAPCs are transfected/pulsed/infected with DNA vectors or mRNAs coding for TAA or neoantigens, followed by immunization of patients.

Advantages of DNA/mRNA-κAPC vaccine: Biopsy is not required. DNA/mRNAs are off-the-shelf; potentially higher efficacy than TAA vaccine using recombinant protein/peptides or synthesize LPs; no need to produce recombinant protein/peptides or synthesize LPs; only PBMC from the patient is needed.

### Example 7

The discovery of Karnelian X reagents and their capability of robustly differentiating cancer monocytes (cMO) into efficacious antigen presentation cells (κAPC) from patients propelled the development of Neo-T™, neoantigen-specific T cells, a technology for treating all cancers.

The core of Neo-T™ is an in vitro setting of κAPC-mediated presentation of tumor antigens, leading to activation and large expansion of tumor-specific T cells (Neo-Ts) from TIL (tumor-infiltrating lymphocytes) for potent tumoricidal activities. For their characteristics, Neo-Ts are activated polyclonal T cells comprising both CD4 and CD8 heterogenicities and with a TCR diversity capable of targeting multiple, if not all, tumor-associated neoantigens and antigens. The success of Neo-T™ technology enables, for the first time, a high-efficacious, cancer type-agnostic, personalized adoptive T cell therapy for cancer elimination.

Neo-T™ technology is a personalized, polyclonal tumor neoantigen/antigen-specific T cell platform. Neo-T™ starts with two materials from the cancer patient: 1) peripheral monocytes (cMo) or PBMC and 2) tumor tissues. PBMC were obtained through leukapheresis. Without further isolation, total PBMC that contain cMo were cultured in Karnelian's proprietary reagent X₁, which differentiates cMo into κAPC in two days. After removal of non-adherent cells, κAPC were further treated with Karnelian X₂ for phenotypic refinement. The final κAPC displayed characteristics of excellent antigen presenting cells, demonstrating elevated proinflammatory markers (IL-12, type I and II IFNs, TNFα, IL-1, IL-6, etc.) (data not shown) and increased expression of antigen presentation machinery for activating both CD4 and CD8 T cells in an immunogenic manner (MHC-I, MHC-II, co-stimulatory molecules CD80, CD86, CD40, OX40L, etc. see FIG. 13B), while maintained low expression of inhibitory molecules that dampen antigen presentation, including SIRPα, Siglec 15, and LilRBs (data not shown). Karnelian X₁

For in vitro expanding Neo-Ts from TIL, tumor tissues were obtained through biopsy and/or surgical resection. A series of physical dissociation and enzymatic digestion was performed to dissociate single cells that comprise alive tumor cells and TIL (tumor-infiltrating lymphocytes). The presence of TIL in single cell dissociated and frequencies of CD4 and CD8 T cells were determined by flow cytometry analyses. As observed in preclinical solid tumor models, TIL including CD4 and CD8 T cells were generally displaying a T_{EM}- or T_{RM}-like memory phenotype (data not shown).

Following tumor dissociation, a portion of alive tumor cells were cultured in the presence of 3T3 feeder cells to establish live tumor cell cultures that are used later for testing Neo-T tumoricidal activities in vitro. The live tumor cell cultures also enabled establishing PDX xenograft models for testing Neo-T tumoricidality in vivo.

Other tumor cells, as well as undigested and digested tumor tissues, were processed by a cycle of freeze-thaw that produces tumor cell necrosis and debris that facilitate κAPC phagocytosis. A period (˜16 h) is given for κAPC uptake, processing and loading of tumor antigens (Ag) to MHC-I/II molecules.

For antigen presentation to call Neo-Ts, tumor dissociated single cells comprising TIL (varied between 5-30%) were added into the culture dishes containing Ag-phagocytosed κAPCs to >70% confluence for co-incubation. This setting of cell proximity allows κAPC conducting antigen presentation to tumor Ag-specific T cells within TIL. In preclinical experiments with KPC pancreatic cancer Ag-loaded κAPC incubating with TIL from established KPC tumors, tumor-specific CD4 and CD8 T cells (Neo-Ts) engaging with κAPC was observed a few hours (2-4 hr) after the co-incubation, and apparent Neo-T activation indicated by the cell size enlargement was observed after an overnight period (˜18h), followed by rapid Neo-T proliferation that generally increases the total T cell numbers over 20-fold (data not shown). IL-2, IL-7 and IL-15 were added to support and promote Neo-T cell expansion. Flow cytometry showed the expanded Neo-Ts comprising both CD4 and CD8 T cells of variable ratios of 30:70-70:30 (different batches), and CD8 T cells displayed high expressions of granzyme B and IFNγ, suggesting potent cytotoxicity (data not shown).

More than 10 sets of human tumor samples and their matched PBMC/peripheral blood samples have been utilized to test autologous Ag-phagocytosed kAPC activating Neo-T in vitro. In all cases, Ag-loaded κAPC exhibited a superior capacity for antigen presentation and induced expansion of a large number of both CD4 and CD8 Neo-Ts from TIL. In vitro and in vivo tumoricidal assays confirmed tumor specificity and potent tumor cell killing ability of Neo-Ts for various tumor kinds (data not shown).

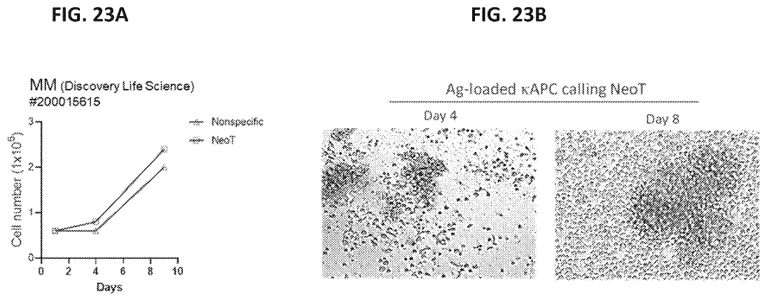

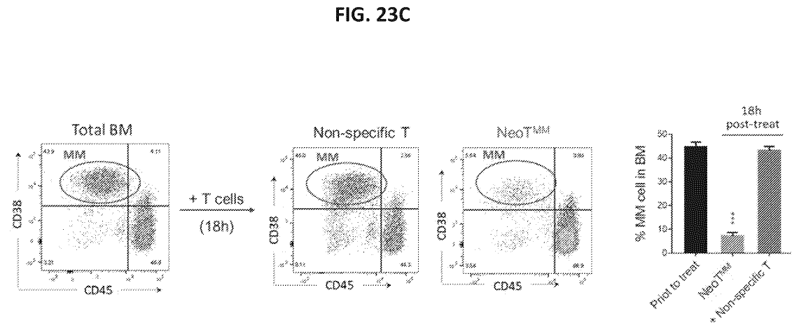

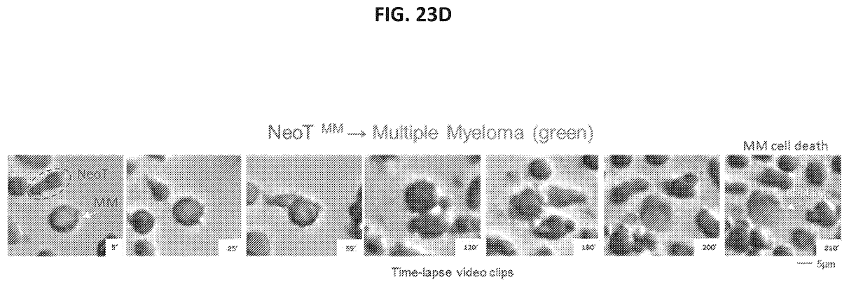

FIGS. 23A-23D demonstrate the ability of κAPC to activate and to prime Neo-Ts to specifically target multiple myeloma cells. Multiple myeloma (MM) patient bone marrow aspirates and matched PBMC were obtained from Discover Life Science, and κAPC were produced from monocytes isolated from patient PBMC. A portion of the bone marrow aspirates underwent one round of freeze/thaw cycle, and the cell debris that comprised cancer (neo) antigens was incubated with κAPC for phagocytosis. After 4 hr incubation, the total bone marrow cells that contained the tumor infiltrating lymphocytes (TILs) was added to the κAPC culture to activate Neo-Ts.

Further, κAPC also were able to activate and prime Neo-Ts against ovarian cancer similarly to multiple myeloma. Fresh ovarian cancer tissue was obtained through surgical resection and dissociated into sing cells. TILs were positively isolated using anti-CD3 antibody-conjugated beads. cMo were derived into κAPC as described above, and the κAPC were incubated with tumor debris from ovarian cancer cells for phagocytosis and antigen presentation, in a similar process as was performed for multiple myeloma, described above.

Comparing autologous Ag-phagocytosed kAPC and anti-CD3/CD28 Ab for activation of Neo-T found that the former method was superior to the latter.

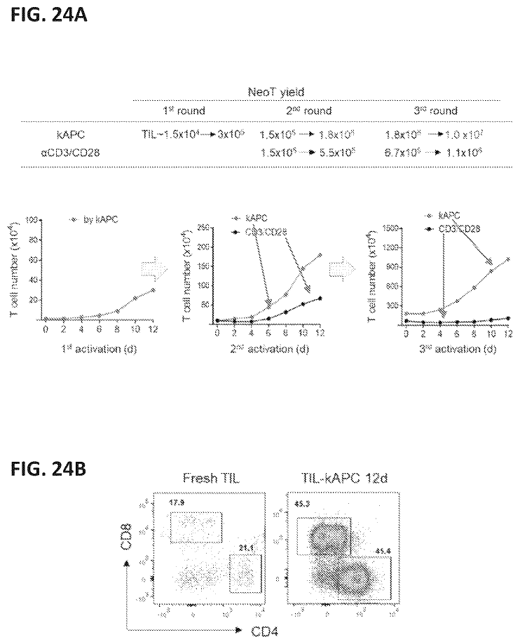

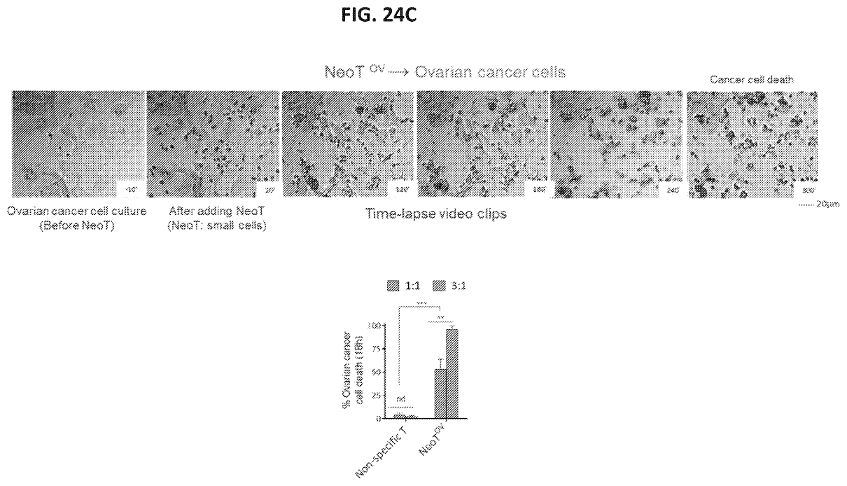

Following the initial step of kAPC-mediated selection and activation of Neo-T, the collected Neo-T were subjected to further expansion using either the same Ag-loaded kAPC that mediate cancer neoantigen/antigen presentation, or anti-CD3 and anti-CD28 antibodies (αCD3/CD28) that ligate TCR. In all cases, Ag-loaded kAPC exhibited a capacity surpassing αCD3/CD28 in the induction of Neo-T expansion (FIG. 24A). We have repeatedly used Ag-loaded kAPC to stimulate Neo-T, every 6-10 days, and observed Neo-T continuous response and expansion, while without showing apparent evidence of exhaustion, even after four rounds. In parallel, Neo-T induced with αCD3/CD28 displayed an overall much weaker response resulting in much less T cell expansion. In addition, αCD3/CD28-stimulated NeoT completely stopped T cell proliferation (senescence) after three rounds of induction, a phenomenon likely due to activation-induced T cell exhaustion/death (mechanism unresolved) (See e.g., Sikora, E. (2015). “Activation-induced and damage-induced cell death in aging human T cells.” Mechanisms of Ageing and Development 151:85-92.). However, this phenomenon of activation-induced cell exhaustion/death was not seen in Ag-phagocytosed kAPC-induced Neo-T.

Moreover, it was found that Ag-loaded kAPC-activated Neo-T generally exhibited stronger cancer cell killing ability than Neo-T activated by αCD3/CD28. Sec e.g., FIG. 24C.

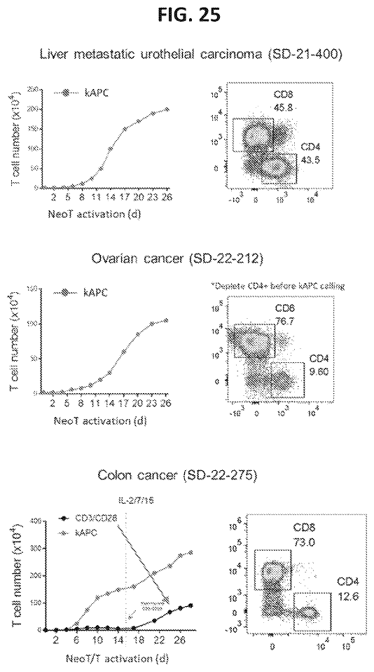

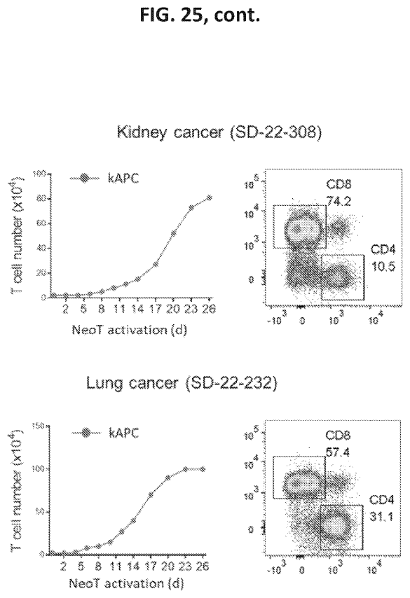

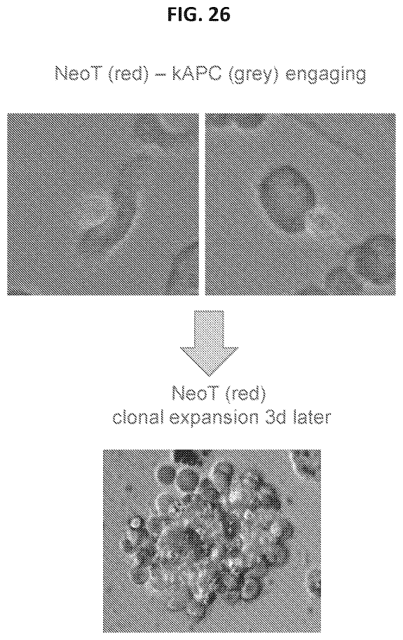

Additional analyses in vitro of the ability of κAPC to activate and prime Neo-Ts against a number of solid tumors was tested as shown in FIG. 25. Across the five tested cancer types, i.e., liver metastatic urothelial carcinoma, ovarian cancer, colon cancer, kidney cancer, and lung cancer, the κAPC-primed Neo-Ts showed similar post-activation T cell expansion. This process of autologous Neo-Ts engaging with κAPC and then activating and clonally expanding is presented in FIG. 26, which provides still images taken from a time-lapse video of κAPC antigen presentation to and engagement with Neo-Ts in vitro.

### Example 8

The strategy of Neo-T was tested in a patient with Uterine Leiomyosarcoma.

Patient information: female, 56 years old Asian, three-year diagnosis of uterine leiomyosarcoma. Treatment history included hysterectomy and chemotherapy that led to remission. The disease was found to recur about 4 months ago and metastases were found in liver, lung, peritoneal cavity and the pelvic area. Since then the patient has gone through immune checkpoint blockade therapy with anti-PD-1 antibody, allogenic NK cell infusion therapy.

Upon admission, CT scanning identified multiple liver metastases. Fine needle aspiration biopsy was performed and obtained 12 cores from two tumor masses. The tissues were subjected to physical dissociation and enzymatic digestion to dissociate single cells that comprise TIL (tumor-infiltrating lymphocytes). After collecting single cells, the rest of tumor tissues were treated with a cycle of free-thaw that produced tumor cell debris for APC phagocytosis.

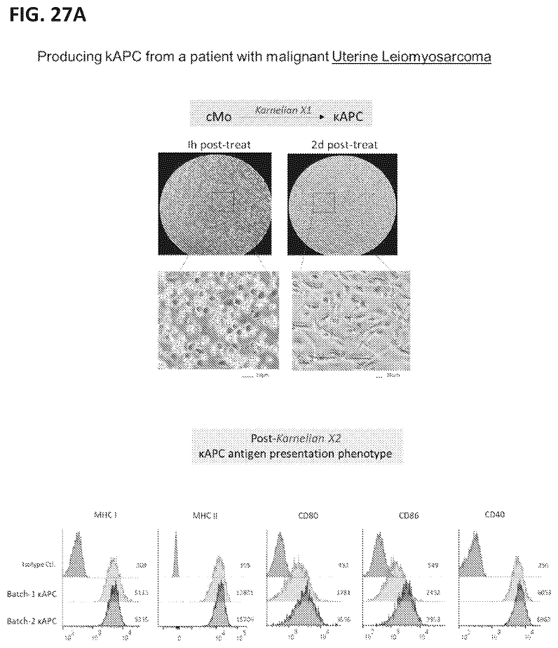

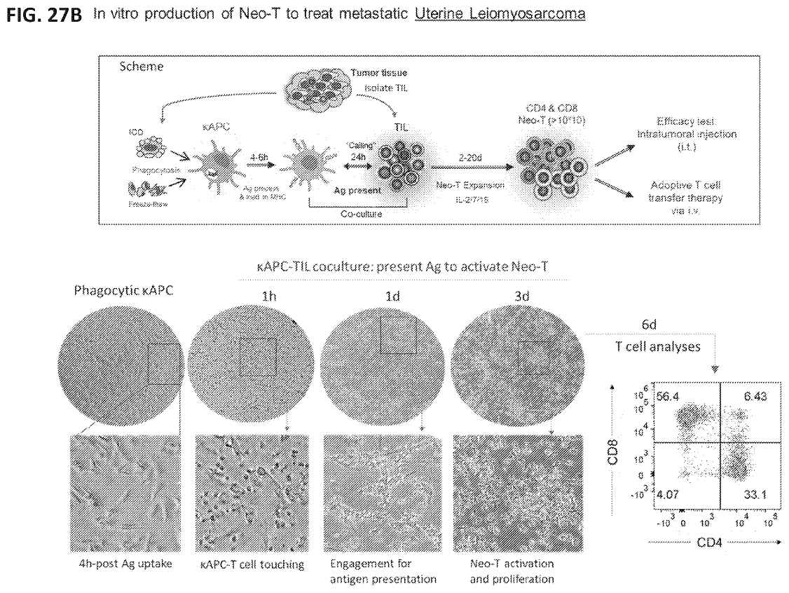

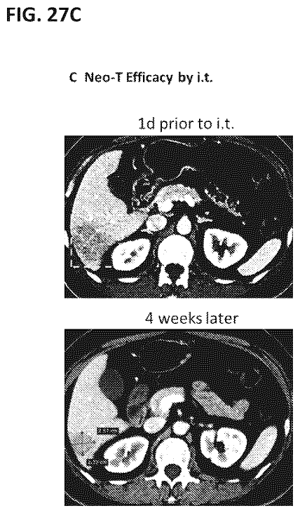

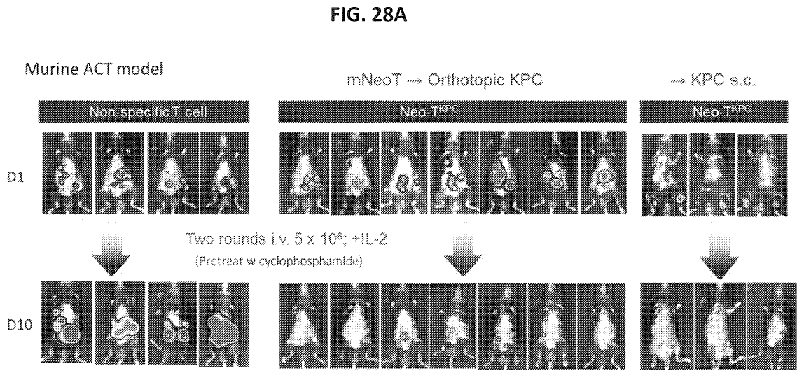

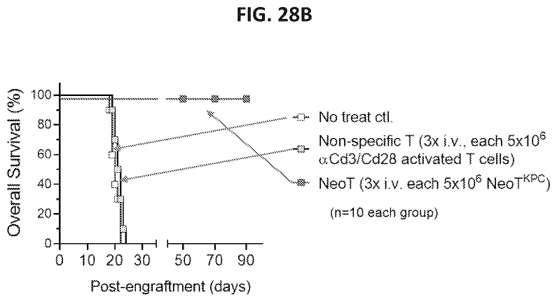

Meanwhile, leukapheresis was conducted and obtained a large number of PBMC (5×10⁹) from which CD14+ monocytes were isolated by negative selection. These monocytes (cMo) were treated with Karnelian X₁ reagent to produced κAPCs (FIG. 27A), followed by a step of phenotypic refinement with Karnelian X₂. The final κAPCs demonstrated an elevated antigen presentation machinery with high expression of MHC-I. MHC-II, CD80, CD86 and CD40 (FIG. 27A).

To produce Neo-T, κAPCs (5×10⁶) were first incubated with free-thaw tumor cells/debris for 6 h, followed by addition of the TIL-containing single cell population (5×10⁶) that were isolated from the tumor biopsies. FIG. 27B depicted the stepwise procedure. As shown in FIG. 27B, κAPCs were observed to be engaged with T cells for antigen presentation soon after the κAPCs-TIL co-culture was initiated, and T cell activation and proliferation were apparent 2 days later. IL-2, IL-7, and IL-15 were added after 3 days to support T cell proliferation with each batch expanded to >10⁹ cells. Flow cytometry of a sample confirmed expansion of both CD4 and CD8 T cells.

The expanded T cells (Neo-Ts) were tested for tumor cell killing by intratumoral injection (2×10⁷ Neo-Ts) into a relatively large metastatic tumor (˜4 cm×3 cm) within the liver. A CT taken 4 weeks later detected reduction of the tumor mass (FIG. 27B).

The NeoT therapy was also tested in a preclinical murine KPC pancreatic ductal adenocarcinoma model. In brief, C57Bl/6 mice were engrafted with KPC pancreatic ductal adenocarcinoma via peritoneal orthotopic engraftment. Following lymphoablation treatment with cyclophosphamide, 5×10⁶ Neo-Ts were injected twice (day 1 and day 5) into KPC tumor-bearing mice. These Neo-Ts were primed in vitro by κAPC that had been loaded with KPC tumor antigens. Tumor volume changes were assessed by the fluorescence readout from each murine tumor, and overall survival was calculated for each group (i.e., 1) no treatment of Neo-Ts; 2) anti-CD3/anti-CD28 stimulation of Neo-Ts; or 3) κAPC stimulation of Neo-Ts). Mice that were given adoptive cellular therapy of κAPC-activated Neo-Ts showed nearly complete regression of tumor burden, and all mice survived up to 90 days post-engraftment, whereas mice given ACT with non-specific Neo-T activation showed limited therapeutic efficacy such that the tumor burden increased and all mice died by day 25 post-engraftment.

1. A method of stimulating a population of monocytes from an individual to produce a population of antigen presenting cells (“APCs”), comprising contacting the population of monocytes with a plurality of survival, differentiation and/or maturation factors (“S/D/M factors”) separately or simultaneously,
wherein the plurality of S/D/M factors comprise: 1) an IL-10 receptor (IL-10R) activator and 2) one or more agents selected from the group consisting of: an IL-4 receptor (IL-4R) activator, a TNFα receptor (TNFR) activator, and an interferon γ (IFNγ) receptor (IFNGR) activator,
thereby obtaining a population of APCs.

2. The method of claim 1, wherein the IL-10R activator is selected from the group consisting of: an IL-10, an IL-10 family member, an IL-10R agonist antibody, a small molecule activator of IL-10R, and an activator of the IL-10R downstream STAT3, optionally wherein the activator of the IL-10R downstream STAT3 is selected from an IL-10 family cytokine, an IL-12 family cytokine, an IL-6 family cytokine, a small molecule STAT3 activator, and G-CSF.

3. The method of claim 1, wherein the IL-10R activator is selected from the group consisting of IL-10, IL-22, IL-19, IL20, IL-24, IL12, IL-23, IL-6, colivelin TFA, Garcinone D, and G-CSF, optionally wherein the IL-10R activator is IL-10, IL-22, IL-19, IL-20, IL-24, IL-12, IL-23, Colivelin TFA, or Garcinone D.

4. The method of any one of claims 1-3, wherein the plurality of S/D/M factors comprise an IL-4R activator, optionally wherein the IL-4R activator is selected from the group consisting of IL-4, an IL-4R agonist antibody, and a small molecule activator of IL-4R.

5. The method of claim 4, wherein the IL-4R activator is IL-4.

6. The method of any one of claims 1-5, wherein the plurality of S/D/M factors comprise a TNFR activator, optionally wherein the TNFR activator is selected from the group consisting of TNFα, a TNFR agonist antibody, and a small molecule activator of TNFR.

7. The method of claim 6, wherein the TNFR activator is TNFα.

8. The method of any one of claims 1-7, wherein the plurality of S/D/M factors comprise an IFNGR activator, optionally wherein the IFNGR activator is selected from the group consisting of IFNγ, an IFNGR agonist antibody, and a small molecule activator of IFNGR.

9. The method of claim 8, wherein the IFNGR activator is IFNγ.

10. The method of any one of claims 1-9, wherein the plurality of S/D/M factors are present in a single composition.

11. The method of any one of claims 1-10, wherein the plurality of S/D/M factors comprise two or more agents selected from the group consisting of an IL-4R activator, a TNFR activator, and an IFNGR activator.

12. The method of claim 11, wherein the plurality of S/D/M factors comprise an IL-10R activator, TNFα, and IFNγ, optionally wherein the plurality of S/D/M factors comprise an IL-10 family cytokine (e.g., IL-10, IL-22, IL-19, IL-24, IL-20, IL-26), TNFα, and IFNγ, optionally wherein the plurality of S/D/M factors comprise an IL-10R activator, IL-4, TNFα, and IFNγ.

13. The method of any one of claims 1-12, wherein the plurality of the S/D/M factors further comprise a GM-CSF receptor (GM-CSFR) activator.

14. The method of claim 13, wherein the GM-CSFR activator is selected from the group consisting of GM-CSF, a GM-CSFR agonist antibody, and a small molecule activator of GM-CSFR.

15. The method of claim 14, wherein the GM-CSFR activator is GM-CSF.

16. The method of any one of claims 1-15, wherein the plurality of the S/D/M factors further comprise an IL-6 receptor (IL-6R) activator, optionally wherein the IL-6R activator is selected from the group consisting of IL-6, an IL-6R agonist antibody, and a small molecule activator of IL-6R.

17. The method of claim 16, wherein the IL-6R activator is IL-6.

18. The method of any one of claims 1-17, further comprising contacting the population of monocytes with a plurality of refinement factors selected from the group consisting of type-I interferon, IFNγ, TNFα, a TLR ligand, CD40L or a CD40-ligating antibody, an anti-PD-L1 antibody, and TPI-1, optionally wherein the type-I interferon comprises IFNα and/or IFNβ, and optionally wherein the TLR ligand is poly IC, CpG, or LPS.

19. The method of claim 18, wherein the refinement factors comprise IFNα, IFNγ, and TNFα.

20. The method of claim 19, wherein the refinement factors further comprise at least two agents selected from the group consisting of poly IC, CpG, CD40L, R848, and an anti-PD-L1 antibody, optionally wherein the refinement factors comprise a SHP-1 inhibitor (e.g., TPI-1).

21. A method of promoting the survival of a population of monocytes from an individual in an in vitro culture, comprising cultivating the population of monocytes in a medium having one or more molecules that promote IL-10 receptor (IL-10R) expression on the monocytes.

20. A method of promoting the survival of a population of monocytes from an individual in an in vitro culture, comprising cultivating the population of monocytes in a medium having an IL-10R activator, optionally wherein the IL-10R activator is selected from the group consisting of: an IL-10, an IL-10 family member, an IL-10R agonist antibody, a small molecule activator of IL-10R, and an activator of the IL-10R downstream STAT3, further optionally the IL-10R activator is IL-10.

21. A method of increasing expression of IL-10 receptor (IL-10R) in a population of monocytes from an individual having cancer, comprising contacting the population of monocytes with one or more agents selected from the group consisting of: an IL-10R activator, a TNFR activator, and an IFNGR activator.

22. A method of promoting the survival of a population of monocytes from an individual in an in vitro culture, comprising cultivating the population of monocytes in a medium comprising IL-10, TNFα, and IFNγ.

23. A method of promoting the differentiation of a population of monocytes from an individual to antigen presenting cells (“APCs”) in an in vitro culture, comprising cultivating the population of monocytes in a medium having one or more molecules selected from the group consisting of an IL-4 receptor (IL-4R) activator, a TNFα receptor (TNFR) activator, and an interferon γ (IFNγ) receptor (IFNGR) activator.

24. The method of any one of claims 1-23, wherein the individual has a cancer.

25. A population of APCs produced by the method of any one of claims 1-20 and 23-24.

26. A population of APCs, wherein the APCs a) are MHC-I+/high, MHC-II+/high, and CD40+/high, b) are TLR2+/high and/or STING+/high, and c) LOX1+/high and/or uPAR+/high, and optionally wherein the expression level of CD40 on the APCs are at least 5-fold, 10-fold, 20-fold, 50-fold, or 100-fold higher than that on monocytes, M1 macrophages, M2 macrophages, and/or MoDCs.

27. A method of activating a population of immune cells, comprising co-culturing the population of immune cells with the population of the APCs of claim 25 or claim 26, wherein the APCs are pre-loaded with one or more neoantigen peptides.

28. The method of claim 27, wherein the method comprises contacting the APCs with a composition comprising a plurality of neoantigen peptides, and/or the APCs have been pre-incubated with the composition.

29. The method of claim 27 or claim 28, wherein the immune cells are selected from the group consisting of PBMC, tumor infiltrating T cells (TIL), and T cells, optionally wherein the immune cells are T cells, optionally wherein the T cells are CD8 T cells and/or CD4 T cells.

30. The method of claim 29, wherein the activating is performed for at least three rounds, wherein each round of co-culture takes at least about 5 days, wherein the co-culture for two of the three rounds do not comprises an anti-CD3 antibody or an anti-CD28 antibody.

31. The method of any one of claims 27-30, wherein the population of immune cells and the antigen presenting cells are derived from the same individual.

32. The method of any one of claims 27-30, wherein the population of immune cells and the antigen presenting cells are not derived from the same individual.

33. A population of activated immune cells obtained by the method of any one of claims 27-32.

34. A method of treating cancer in a patient, comprising administering to the patient a population of APCs of claim 25 or claim 26 and/or activated immune cells of claim 33.

34. A composition comprising a plurality of survival, differentiation and/or maturation factors (“S/D/M factors”), wherein the plurality of S/D/M factors comprise: 1) an IL-10 receptor (IL-10R) activator and 2) one or more agents selected from the group consisting of: an IL-4 receptor (IL-4R) activator, a TNFα receptor (TNFR) activator, and an interferon γ (IFNγ) receptor (IFNGR) activator.

35. The composition of claim 34, wherein the IL-10R activator is selected from the group consisting of: an IL-10, an IL-10 family member, an IL-10R agonist antibody, a small molecule activator of IL-10R, and an activator of the IL-10R downstream STAT3.

36. The composition of claim 35, wherein the IL-10R activator is IL-10, IL-22, IL-19, IL-20, IL-24, IL-12, IL-23, Colivelin TFA, or Garcinone D, optionally wherein the IL-10R activator is IL-10.

37. The composition of claim 35 or claim 36, wherein the plurality of S/D/M factors comprise two or more agents selected from the group consisting of an IL-4R activator, a TNFR activator, and an IFNGR activator.

38. The composition of claim 37, wherein the plurality of S/D/M factors comprises IL-10, IL-4, TNFα, and IFNγ.

39. The composition of any one of claims 35-38, wherein the plurality of the S/D/M factors further comprise a GM-CSF receptor (GM-CSFR) activator.
